(19) United States
(12) Patent Application Publication
(10) Pub. No.: US 2026/0235605 A1
(43) Pub. Date: Aug. 13, 2026

(54) BIOSIGNATURE INTEGRATION TO ASSESS THERAPY RESPONSE IN BREAST CANCER

(71) Applicant: PRELUDE CORPORATION, Laguna Hills, CA (US)

(72) Inventors: Karuna Mittal, Cumming, GA (US); Troy Bremer, Irvine, CA (US)

(21) Appl. No.: 19/153,516

(22) PCT Filed: Feb. 13, 2024
(86) PCT No.: PCT/US2024/015652

§ 371 Date: Aug. 4, 2025

Related U.S. Application Data
(60) Provisional application No. 63/484,966, filed on Feb. 14, 2023.

(51) Int. Cl. G01N 33/57515; A61N 5/10; G01N 21/64; G01N 33/58

(52) U.S. Cl. CPC G01N 33/57515; A61N 5/103; G01N 21/6428; G01N 21/6456; G01N 33/582; G01N 2021/6439; G01N 2333/4706; G01N 2333/70521; G01N 2333/90203

(57) ABSTRACT

A method of determining a risk of recurrence for breast cancer is provided, the method including obtaining a sample provided by a subject, wherein the sample is characterized as estrogen receptor and progesterone receptor positive, and human epidermal growth factor 2 negative; assaying the sample for markers, the markers comprising programmed cell death protein 1 and interferon regulatory factor 9; and assessing the subject as having a significant risk of recurrence if the assaying step indicates elevated levels of expression of PD-1 and IRF9 in the sample; and assessing the subject as having no significantly increased risk of recurrence if the assaying step indicates levels of expression lower than the elevated levels of expression for the PD-1 and IRF9.

### RELATED APPLICATIONS

The present application claims priority to U.S. Provisional Application No. 63/484,966, filed Feb. 14, 2023, the content of which is incorporated herein by reference in its entirety.

### BACKGROUND

The present technology generally relates to whether or not a subject who has breast cancer will be responsive to standard (or some other form of) radiotherapy in terms of recurrence, e.g., local recurrence, of breast cancer based on results from the analysis of one or more markers.

### SUMMARY

The methods disclosed herein each have several aspects, no single one of which is solely responsible for their desirable attributes. Without limiting the scope of the claims, some prominent features will now be discussed briefly. Numerous other embodiments are also contemplated, including embodiments that have fewer, additional, and/or different components, steps, features, objects, benefits, and advantages. The components, aspects, and steps may also be arranged and ordered differently. After considering this discussion, and particularly after reading the section entitled “Detailed Description”, one will understand how the features of the devices and methods disclosed herein provide advantages over other known devices and methods.

As provided herein, a method of determining a risk of recurrence for breast cancer, the method including obtaining a sample provided by a subject, wherein the sample is characterized as estrogen receptor (ER) and/or progesterone receptor (PR) positive, and human epidermal growth factor 2 (HER2) negative; assaying the sample for markers, the markers comprising programmed cell death protein 1 (PD-1) and interferon regulatory factor 9 (IRF9); and assessing the subject as having a significant risk of recurrence if the assaying step indicates elevated levels of expression of PD-1 and IRF9 in the sample; assessing the subject as having no significantly increased risk of recurrence if the assaying step indicates levels of expression lower than the elevated levels of expression for the PD-1 and IRF9.

In some embodiments, a method for treating breast cancer is provided herein, the method including obtaining a sample provided by a subject, wherein the sample is characterized as ER and PR positive, and HER2 negative; assaying the sample for markers, the markers comprising PD-1 and IRF9; and providing an RT boost to the subject if the assaying step indicates elevated levels of expression of PD-1 and IRF9 in the sample; and de-escalating the RT of the subject if the assaying step indicates levels of expression lower than the elevated levels of expression for the PD-1 and IRF9.

In some embodiments, a method for treating breast cancer is provided herein, the method including providing a sample, wherein the sample is characterized as ER and PR positive, and HER2 negative; receiving an RT boost if the sample includes elevated levels of expression of PD-1 and IRF9; and de-escalating an RT if the sample includes levels of expression of PD-1 and IRF9 that lower than the elevated levels of expression for the PD-1 and IRF9.

### BRIEF DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1 depicts a flow diagram of a method according to one of the embodiments.

FIG. 2 depicts a flow diagram of a method according to one of the embodiments.

FIG. 3 depicts a flow diagram of a method according to one of the embodiments.

FIGS. 4A-4I show fluorescent images of a tissue microarray (TMA) section from an F3 core. FIG. 4A shows five different markers labeled in the imaged section, which include PDL-1 (Opal™ 520), PD-1 (Opal™ 620), ER (Opal™ 690), PR (Opal™ 480), and CK (Opal™ 570). FIG. 4B shows an ER-positive immunofluorescence using Opal™ 690, which is made clearer in the IHC view presented by FIG. 4C. FIG. 4D shows a lack of PR signal from Opal™ 480, which is made clearer in the IHC view presented by FIG. 4E. FIG. 4F shows PR-positive immunofluorescence using Opal™ 480, which is made clearer in the IHC view presented by FIG. 4G. FIG. 4H shows a PD-1 positive immunofluorescence using Opal™ 620, which is made clearer in the IHC view presented by FIG. 4I.

FIGS. 5A-5E show fluorescent images of a TMA section from an F3 core. FIG. 5A shows five different markers labeled in the imaged section, which include ALDHA1 (Opal™ 520), IRF-9 (Opal™ 570), HIF1A (Opal™ 690), Ki67 (Opal™ 620), and PanCK (Opal™ 480). FIG. 5B shows ALDHA1-positive immunofluorescence using Opal™ 520, which is made clearer in the IHC view presented by FIG. 5C. FIG. 5D shows IRF9 positive immunofluorescence, which is made clearer in the IHC view presented by FIG. 5E.

FIGS. 6A-6G show fluorescent images of a TMA section from an F3 core. FIG. 6A shows five different markers labeled in the imaged section, which include GLUT1 (Opal™ 570), HER2 (Opal™620), p16/INK4A (Opal™ 520), SIAH2 (Opal™ 690), and CK (Opal™ 480). FIG. 6B shows GLUT1-positive immunofluorescence using Opal™ 570, which is made clearer in the IHC presented by FIG. 6C. FIG. 6D shows P16-positive immunofluorescence using Opal™ 520, which is made clearer in the IHC presented by FIG. 6E. FIG. 6F shows HER2-positive immunofluorescence using Opal™ 620, which is made clearer in the IHC presented by FIG. 6G.

### DETAILED DESCRIPTION

All patents, applications, published applications and other publications referred to herein are incorporated herein by reference to the referenced material and in their entireties.

Multiple methodologies distinguish tumor versus stroma areas/compartments on a tissue section. The most widely used method is a manual process where a trained pathologist applies their expertise in histology on H&E-stained tissue sections to distinguish tumor and the surrounding stromal areas. However, this process is extremely time consuming, and (worryingly) the accuracy of the identified areas for such tissue sections varies between pathologist to pathologist. Moreover, it is very challenging to identify and annotate with precision carcinoma cells and stromal cells within tumor islands, at the tumor/stroma interface, and more distant stromal cell populations that are devoid of carcinoma cells. Therefore, using manually created data (e.g., pathologist annotated data) to train the models for identifying tumor and stromal cells with greater precision and accuracy along with the ability to interrogate multiple markers concurrently is crucial.

### Definitions

The term “and/or” shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising a nucleic acid molecule” includes single or plural nucleic acid molecules and is considered equivalent to the phrase “comprising at least one nucleic acid molecule.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. Unless otherwise specified, the definitions provided herein control when the present definitions may be different from other possible definitions.

As used herein, the term “subject” encompasses any animal including humans, preferably a mammal. Exemplary subjects include but are not limited to humans, primates, livestock (e.g. sheep, cows, horses, donkeys, pigs), companion animals (e.g. dogs, cats), laboratory test animals (e.g. mice, rabbits, rats, guinea pigs, hamsters), captive wild animals (e.g. fox, deer). Preferably the mammal is a human or primate. More preferably the mammal is a human. “Subject” and “patient” are used interchangeably herein.

As used herein, the term “diagnosis”, and variants thereof, such as, but not limited to “diagnose” or “diagnosing” shall include, but not be limited to, a primary diagnosis of a clinical state or any primary diagnosis of a clinical state. Furthermore, this term denotes the process of identifying a disease by its signs, symptoms and results of various tests. The conclusion reached through that process is also called “a diagnosis.” Forms of testing commonly performed include biopsy for the collection of the tumor. In some embodiments, the prognosis can be a high or low likelihood of a subsequent (within the next 10 years, 15, or 20 years) invasive breast cancer event.

As used herein, the term “prognosis” denotes an outcome or course of a disease. In some embodiments provided herein, the phrase, when used in the context of a person already having invasive breast cancer, denotes the likelihood that a subject having the invasive breast cancer will go on (within a following ten, fifteen, or twenty year period) to have a subsequent ipsilateral invasive breast cancer event after surgical removal of the primary tumor. The outcome can include a) the likelihood of an ipsilateral breast event, b) the likelihood of an ipsilateral breast event in a particular amount of time (e.g., 1, 2, 3 or 5 years), c) the likelihood that a particular therapy (e.g., radiation) will prevent an ipsilateral breast event, d) an optimal treatment to help prevent an ipsilateral event that matches the severity of the most likely event, or e) combinations thereof.

As used herein, the term “breast tumor” denotes a neoplastic condition of breast tissue that can be benign or malignant. The term “tumor” is synonymous with “neoplasm” and “lesion”. Exemplary breast tumors include invasive breast cancer, ductal carcinoma in situ (DCIS), lobular carcinoma in situ (LCIS), atypical ductal hyperplasia (ADH).

As used herein, the term “cancer” denotes a malignant neoplasm that has undergone characteristic anaplasia with loss of differentiation, increased rate of growth, invasion of surrounding tissue, and is capable of metastasis. The term “cancer” shall be taken to include a disease that is characterized by uncontrolled growth of cells within a subject, such as, but not limited to, invasive breast cancer. In some embodiments, invasion of the surrounding tissue is the invasion of the basement membrane.

As used herein, the term “intraductal lesion” refers to tumors that are confined to the interior of the mammary ducts and are, therefore, not invasive breast cancers. Exemplary intraductal lesions include ADH and DCIS.

As used herein, ADH is a neoplastic intraductal (non-invasive) lesion characterized by proliferation of evenly distributed, monomorphic mammary epithelial cells.

As used herein, DCIS is a neoplastic intraductal (non-invasive) lesion characterized by increased mammary epithelial proliferation with subtle to marked cellular atypia. DCIS has been divided into grades (low, intermediate, and high) based on factors such as nuclear atypia, intraluminal necrosis, mitotic activity etc. Low-grade DCIS and ADH are morphologically identical, and ADH is distinguished from DCIS based on the extent of the lesion, as determined by its size and/or the number of involved ducts. DCIS is initially typically diagnosed from a tissue biopsy triggered by a suspicious finding (e.g., microcalcifications, unusual mass, tissue distortion or asymmetry, etc.) on a mammogram and/or ultrasound imaging test. It may be from routine screening imaging or, more rarely, from diagnostic imaging triggered by a positive physical examination (e.g., a palpable mass, nipple discharge, skin change, etc.) or by a significant change in a previously identified mass.

Cellular proliferation in DCIS is confined to the milk ducts. If the proliferating cells have invaded through the basement membrane of the myoepithelial cell (MEC) layer lining the duct, thus appearing in the surrounding stroma, then the lesion is considered an invasive breast cancer, even if DCIS is also present. In some cases, the invasion is very minimal (microinvasion) or the only evidence of invasion is disruption of the MEC layer (e.g., by observing discontinuities in MEC-specific protein marker stains such as SMMHC and/or p63). Typically, these microinvasive cases are treated as invasive breast cancers, although there is some controversy in the treatment of these cases.

Recurrence rates in DCIS with current treatments are difficult to estimate. However, it is likely that about 20% of patients who receive lumpectomies without any further treatment would experience recurrence events within 10 years, approximately evenly split between DCIS and invasive events, while about 5% of patients who receive nipple sparing skin sparing mastectomies would experience recurrence. Standard of care with lumpectomy is to receive adjuvant radiation therapy (RT). Several randomized clinical trials provide evidence that adjuvant radiation therapy following lumpectomy reduces recurrence risk by approximately half for both DCIS and invasive event types, and that current clinical and pathologic assessment techniques cannot identify a low-risk sub-group in which there is no benefit from radiation therapy.

As used herein, the term “invasive breast cancer” denotes that the neoplastic (tumor) cells have invaded through the epithelial basement membrane. This distinguishes invasive breast cancer from other hyperplastic (ductal hyperplasia) or dysplastic (atypical ductal hyperplasia, ADH) or non-invasive neoplastic (DCIS, LCIS) breast lesions which are characterized by an intact (non-invaded) basement membrane. It can be divided into stages (I, IIA, IIB, IIIA, IIIB, and IV). In some embodiments, any of the methods provided herein can be applied to invasive breast cancer to determine the success of radiotherapy for preventing an invasive breast cancer recurrence. In some embodiments, any of the methods provided herein can be applied to DCIS to determine the success of radiotherapy for preventing a DCIS cancer recurrence.

As used herein, the term “invasive breast cancer” denotes that the neoplastic (tumor) cells have invaded through the epithelial basement membrane. This distinguishes invasive breast cancer from other hyperplastic (ductal hyperplasia) or dysplastic (atypical ductal hyperplasia, ADH) or non-invasive neoplastic (DCIS, LCIS) breast lesions which are characterized by an intact (non-invaded) basement membrane. It can be divided into stages (I, IIA, IIB, IIIA, IIIB, and IV). It can be divided into different molecular subtypes based on the presence of Estrogen receptor, Progestrone receptor and human epidermal growth factor 2 (HER2) in/on the tumor cells. In some embodiments, any of the methods provided herein can be applied to invasive breast cancer to determine the success of radiotherapy for preventing an invasive breast cancer recurrence. Rates of recurrence in invasive breast cancer varies between 3-10% post BCS+RT. Meta analysis of the landmark randomized clinical trials provide evidence that adjuvant radiation therapy following lumpectomy reduces recurrence risk by approximately half. It further demonstrated that the absolute benefits of radiotherapy varied between subgroups defined by age, hormone receptor status, and other risk factors suggesting that the current clinical and pathologic assessment techniques cannot identify a low-risk sub-group in which there is no benefit from radiation therapy.

As used herein, the terms “HER2+ve” and/or “HER2 enhanced” breast cancer refers to breast cancers wherein a least a portion of the cancer cells overexpress human epidermal growth factor 2 (HER2) on the surface of the cancer cells. HER2 is a receptor tyrosine kinase involved in the regulation of cell growth and differentiation. HER2+ve breast cancers are characterized by the enhanced HER2 expression assayed using IHC or FISH than the invasive breast cancers that are not HER2+ve.

As used herein, the term “HR+ve” breast cancer refers to breast cancers wherein ER and/or PR are present on the surface of cancer cells.

As used herein, the term “radiation therapy” (RT) denotes a therapy that involves or includes some form of radiation in an amount that is therapeutic to the subject.

As used herein, the terms “RT boost” or “boost,” the latter when used within the context of RT, includes an additional/extra dose of radiation applied to a site. The site may be a tumor bed. The site may be an initial tumor site. The site may be any tissue or location requiring the additional or extra dose of radiation. The additional/extra dose may be simultaneously integrated/concomitant (i.e., given together with the standard radiotherapy) or sequential (i.e., given after standard radiotherapy). The additional/extra dose of radiation may be at least 10 Gy if given sequentially or at least 5 Gy if simultaneously integrated/concomitant. The additional/extra dose of radiation may be no more than 16 Gy. The additional/extra dose may be provided using external beam radiotherapy, internal beam radiotherapy/brachytherapy, stereotactic radiotherapy, or intraoperative radiotherapy.

Surgery is a treatment for a breast tumor and is frequently involved in diagnosis. The type of surgery depends upon how widespread the tumor is when diagnosed (the tumor stage), as well as the type and grade of tumor.

The terms “treatment” and “therapy” as provided herein are used interchangeably and does not require the complete or 100% curing of the subject. Instead, it encompasses the broader concept or delaying the onset of one or more symptoms, extending the life and/or quality of life of the subject, reducing the severity of one or more symptoms, etc.

“Risk of invasive breast cancer,” denotes a risk of developing (or being diagnosed with) a subsequent invasive breast cancer in the same (a.k.a. ipsilateral) breast.

Adjuvant chemotherapy is often used after surgery to treat any residual disease. Systemic chemotherapy often includes a platinum derivative with a taxane. Adjuvant chemotherapy is also used to treat subjects who have a recurrence or metastasis.

“Adjuvant invasive breast cancer treatment” denotes any treatment that is appropriate for a subject that is likely to have an invasive breast cancer occurrence, which can include, lumpectomy with radiation, to lumpectomy with a receptor targeted chemotherapy, to lumpectomy with radiation with a receptor targeted chemotherapy, to mastectomy, to mastectomy with a receptor targeted chemotherapy, to mastectomy with radiation, to mastectomy with radiation and a receptor targeted chemotherapy, to surgery with a chemotherapy. In some embodiments, the targeted therapy can include: endocrine therapy (blocking hormone receptors); anti-HER2 therapy (blocking the HER2 receptor which is overexpressed in some breast cancers), immunotherapy (blocking molecules which inhibit the immune response); conjugated monoclonal antibodies (an antibody conjugated to chemotherapy (the antibody will function as a homing device to bring the chemotherapy specifically to the cancer cells); tyrosine kinase inhibitors (receptors relying on tyrosine kinase signaling); anti-PDGFRb antibodies (blocks PDGFRb); PARP inhibitor therapy (blocking poly ADP ribose polymerase (PARP) activity), etc.

As used herein, “chemotherapy” (CT) refers to one kind of treatment for treating cancers, wherein a therapeutic amount of one or more chemotherapeutic agents is administered to target and destroy cancerous cells, particularly those that have proliferated beyond the primary tumor site and metastasized at distant locations within the subject's body.

“Standard of care” or “standard” as used herein, with reference to a therapy or treatment, have the ordinary and customary meaning to one of ordinary skill in the art in view of the present disclosure. In some embodiments, standard of care denotes a therapy or treatment option recommended for a patient under a guideline such as that provided by NCCN, ESMO, ESTRO, ASTRO, Clinical Practice Recommendations Australia, or NICE guideline, and optionally, any one or more of the respective guidelines as of June 2022. In some embodiments, a therapy recommended under standard of care for a patient does not take into account guidance provided by analysis of markers as disclosed herein.

The terms “standard radiation therapy” and “standard radiotherapy” are used interchangeably herein and denote a therapy that involves or includes some form of radiation in an amount that is therapeutic to the subject under the current standard of care for breast cancer. In some embodiments, the standard of care is any one that is provided in NCCN, ESMO, ESTRO, ASTRO, Clinical Practice Recommendations Australia, or NICE guideline, and optionally, any one or more of the respective guidelines as of June 2022. In some embodiments, the standard of care is any one of those provided in Tables 1 and 2 below.

TABLE 1
Radiotherapy Guidelines

| | N- | | | | |
|---|---|---|---|---|---|
| Guideline | status | Surgery | Volume | Fractionation | Boost (tumor bed) |
| NCCN | N0 | BCS | WBRT * | 40-42.5 Gy in 15-16 | 10-16 Gy in 4-8 fraction |
| | | | and ** | fractions (or 45-50.5 | if high risk *** |
| | | | | Gy in 25-28 Fr.) | |
| NCCN | N+ | BCS | WBRT + RNI | 40-42.5 Gy in 15-16 | 10-16 Gy in 4-8 fraction |
| | | | | fractions (or 45-50.5 | if high risk *** |
| | | | | Gy in 25-28 Fr.) | |
| ESMO | N0 | BCS | WBRT§ | 45-50 Gy in 25-28 | 10-16 Gy in 4-8 fraction |
| | | | | fractions (or 2.5-2.67 | if high risk *** |
| | | | | Gy X 15-16 Fr) | |
| ESMO | N+ | BCS | WBRT + RNI | 45-50 Gy in 25-28 | 10-16 Gy in 4-8 fraction |
| | | | | fractions (or 2.5-2.67 | if high risk *** |
| | | | | Gy X 15-16 Fr) | |
| NICE | N0 | BCS | WBRT§ | 40 Gy in 15 fractions | 10 Gy in 5 fractions if |
| | | | | | high risk z |
| NICE | N+ | BCS | WBRT + RNI | 40 Gy in 15 fractions | 10 Gy in 5 fractions if |
| | | | | | high risk z |

WBRT = Whole breast radiotherapy
RNI = regional nodal irradiation
BCS = breast-conserving surgery
N0 = no lymph node involvement
N+ = lymph node involvement
* APBI if low risk
** omission considered if >70 ER+, T1, Endocrine Treatment (ET)
*** >2 cm, younger age, LVI (lymphovascular invasion)
§APBI if age <50, T <=3, ER-positive, HER2-negative and grade 1 to 2, non-lobular
z Risk can be estimated using a range of standardised tools and clinical expertise
NCCN Guidelines Version 4.2021 (Invasive Breast Cancer)
PRINCIPLES OF RADIATION THERAPY
Optimizing Delivery of Individual Therapy
It is important to individualize RT planning and delivery.
CT-based treatment planning should be routinely utilized to delineate target volumes and
adjacent organs at risk.
Radiation to the breast/chest wall and nodal regions is generally delivered with single
energy or mixed energy photons± electrons.
Improved homogeneity of the target dose and sparing of normal tissues can be
accomplished using compensators such as wedges, forward planning using segments,
and intensity-modulated RT (IMRT).
Additional techniques such as respiratory control (deep inspiration breath-hold), prone
positioning, cardiac blocks may also be used to try to further reduce dose to heart, lung,
and adjacent normal tissue.
Verification of treatment setup consistency is done with weekly imaging. When using
certain techniques (i.e., prone breast), more frequent imaging may be appropriate.
Standard utilization of daily imaging is not recommended.
When treating the internal mammary nodes, dose-volume histograms (DVHs) should be
used to evaluate dose constraints, dose to normal tissues (i.e., heart, lung), and planning
target volumes (PTVs).
It is common for RT to follow chemotherapy when chemotherapy is indicated.
Whole Breast Radiation
Target definition is the breast tissue in entirety.
RT dosing:
The whole breast should receive a hypofractionated dose of 40-42.5 Gy in 15-16
fractions; in selected cases 45-50.4 Gy in 25-28 fractions may be considered.
A boost to the tumor bed is recommended in patients at higher risk for recurrence.
Typical boost doses are 10-16 Gy in 4-8 fractions.
Lumpectomy cavity boost can bedelivered using enface electrons, photons, or
brachytherapy.
For patients who require a more limited number of treatment visits for WBRT delivery,
ultra-hypofractionated WBRT of 28.5 Gy delivered as 5 (once-a-week) fractions, may
be considered in selected patients aged ≥50 years following BCS with pTis/T1/T2/N0
tumor. However, late toxicity effects beyond 10 years are not currently defined.
The optimal fractionation for the delivery of a boost is not known for this regimen.
3-D planning to minimize inhomogeneity and exposure to heart and lung is essential
when using this regimen.
ESMO guidelines
Whole-breast radiotherapy.
RT after BCS: Postoperative RT is strongly recommended after BCS [I, A]. WBRT alone reduces
the 10-year risk of any first recurrence (including locoregional and distant) by 15% and the 15-year
risk of breast cancer-related mortality by 4%. Boost RT gives a further 50% RR reduction and is
indicated for most patients who have unfavourable risk factors for local control such as age <50
years, grade 3 tumours, presence of vascular invasion or extensive intraductal component and non-
radical tumour excision (focally-otherwise further surgery should be advocated) [I, A].
Recommendations:
Postoperative RT is strongly recommended after BCS [I, A].
Boost RT is recommended to reduce the risk of in-breast re-lapse in patients at higher risk
of local recurrence [I, A].
Accelerated partial-breast RT after BCS: The concept of accelerated partial-breast irradiation (APBI)
is an appealing approach to substantially shorten the overall treatment time. The rationale for APBI
is that the majority of local failures occur in the vicinity of the primary tumour site, while so-called
‘elsewhere’ in-breast failures may represent a new primary tumour. Excellent results with low local
recurrence rates equivalent to WBRT are reported for partial-breast irradiation (accelerated and
conventionally fractionated) using external beam techniques and brachytherapy. However, for
intraoperative RT, as used in the ELIOT (single dose with electrons) and TARGIT (single dose with
50-kV X- rays) randomised trials, the ipsilateral breast cancer recurrence rate was significantly
higher in the APBI groups, compared with the WBRT. Based on these results, APBI might be
considered an acceptable treatment option in patients with a low risk for local recurrence, for example
those who are at least 50years old, with unicentric, unifocal, node-negative, non- lobular breast
cancer, up to 3 cm without the presence of extensive intraductal components or vascular invasion
and with negative margins, especially if they will receive adjuvant endocrine treatment [III, C]. APBI
may also be considered for low-grade DCIS [III, C]. More and long-term results of several past and
ongoing prospective randomised APBI trials are awaited.
Recommendation:
APBI is an acceptable treatment option in patients with a low risk for local recurrence [III,
C].
Early and locally advanced breast cancer: diagnosis and management
NICE guideline [NG101] Published: 18 Jul. 2018
Radiotherapy after breast-conserving surgery
1.10.3 Offer whole-breast radiotherapy to women with invasive breast cancer who have had
breast-conserving surgery with clear margins. [2018]
1.10.4 Consider partial breast radiotherapy (as an alternative to whole-breast radiotherapy) for
women who have had breast-conserving surgery for invasive cancer (excluding lobular type)
with clear margins and who:
have a low absolute risk of local recurrence (defined as women aged 50 and over with
tumours that are 3 cm or less, N0, ER-positive, HER2-negative and grade 1 to 2) and
have been advised to have adjuvant endocrine therapy for a minimum of 5 years. [2018]
1.10.5 When considering partial breast radiotherapy (see recommendation 1.10.4), discuss the
benefits and risks, and explain that:
local recurrence with partial breast radiotherapy at 5 years is equivalent to that with
whole-breast radiotherapy
the risk of local recurrence beyond 5 years is not yet known
there is a potential reduction in late adverse effects. [2018]
1.10.6 When delivering partial breast radiotherapy, use external beam radiotherapy. [2018]
1.10.7 Consider omitting radiotherapy for women who:
have had breast-conserving surgery for invasive breast cancer with clear margins and
have a very low absolute risk of local recurrence (defined as women aged 65 and over with
tumours that are T1N0, ER-positive, HER2-negative and grade 1 to 2) and
are willing to take adjuvant endocrine therapy for a minimum of 5 years. [2018]
1.10.8 When considering omitting radiotherapy for the population in recommendation 1.10.7,
discuss the benefits and risks... and explain that:
without radiotherapy, local recurrence occurs in about 50 women per 1,000 at 5 years, and
with radiotherapy, occurs in about 10 women per 1,000 at 5 years
overall survival at 10 years is the same with or without radiotherapy
there is no increase in serious late effects if radiotherapy is given (for example, congestive
cardiac failure, myocardial infarction or secondary cancer). [2018]
Dose fractionation
1.10.13 Use external beam radiotherapy giving 40 Gy in 15 fractions as standard practice for
women with invasive breast cancer after breast-conserving surgery or mastectomy. [2009]
Breast boost following breast-conserving surgery
1.10.14 Offer an external beam boost to the tumour bed for women with invasive breast cancer
and a high risk (www.nice.org.uk/guidance/ng101/chapter/Recommendations) of local
recurrence, following whole-breast radiotherapy. [2009, amended 2018]
1.10.15 Inform women of the risk of side effects associated with an external beam boost to the
tumour bed following whole-breast radiotherapy. [2009, amended 2018]
Radiotherapy to nodal areas
1.10.16 Do not offer adjuvant radiotherapy to regional lymph nodes to people with invasive
breast cancer who have been shown to have histologically lymph node-negative breast
cancer. [2009, amended 2018]
1.10.17 Do not offer adjuvant radiotherapy to the axilla after axillary clearance for invasive
breast cancer. [2009, amended 2018]
1.10.18 Offer adjuvant radiotherapy to the supraclavicular fossa to people with invasive breast
cancer and 4 or more involved axillary lymph nodes. [2009]
1.10.19 Offer adjuvant radiotherapy to the supraclavicular fossa to people with invasive breast
cancer and 1 to 3 positive lymph nodes if they have other poor prognostic factors (for example,
T3 and/or histological grade 3 tumours) and good performance status. [2009]
1.10.20 Consider including the internal mammary chain within the nodal radiotherapy target for
people with node-positive (macrometastases) invasive breast cancer. [2018]
ESTRO
Ultra-hypofractionation (26 Gy in five fractions) for chest wall irradiation without breast
reconstruction can be offered as standard of care or within a randomised controlled trial or
prospective registration cohort.
International guidelines
The following international guidelines have been identified which include guidance on the use of
hypofractionated radiotherapy for early breast cancer:
The American Society for Radiation Oncology (ASTRO) guidelines on fractionation for
whole breast irradiation, 2010
The New Zealand Ministry of Health Guidelines for Management of Early Breast Cancer, 2009
NICE Guidelines for early and locally advanced breast cancer, 2009
Scottish Intercollegiate Guidelines Network (SIGN) guidelines, 2009
BC Cancer Agency Breast cancer management consensus guidelines 2013
European Journal of Medical Oncology (ESMO) guidelines on primary breast cancer
diagnosis, treatment and follow-up, 2013
German Society of Radiation Oncology (DEGRO) guidelines on radiotherapy of breast
cancer, 2013
Nice-Saint-Paul de Vence guidelines on adjuvant radiotherapy in the management of axillary
node negative invasive breast cancer, 2013
The BC Cancer Agency consensus-based guidelines for the management of early breast cancer
include recommendations on the use of radiotherapy and recommend a hypofractionated
radiotherapy regimen as standard. The guideline recommends the following dose fractionation for
radiotherapy following breast conserving therapy (T1, T2; N0):
1. Standard whole breast dose is 42.5 Gray (Gy) in 16 daily fractions
2. Certain patients are at risk for inferior cosmetic outcome from the 16-fraction course.
Extended fractionation should be considered for patients with very large breast size, and
those with significant post-operative induration, oedema, erythema, hematoma or infection.
Patients with these indications for extended fractionation should receive 45Gy in 25 daily
fractions plus a boost dose of 10Gy in 5 fractions or 50.4 Gy in 28 daily fractions.
3. If a boost is used, an additional dose of 6-16 Gy in 3-8 fractions is recommended.

TABLE 2
ESTRO Radiotherapy Guidelines (2022)
Panel: Final consensus statements

| | |
|---|---|
| 1. | Whole breast irradiation |
| a | Moderate hypofractionated whole breast irradiation should be offered regardless of age at breast |
| | cancer diagnosis, pathological tumour stage, breast cancer biology, surgical margins status, |
| | tumour bed boost, breast size, invasive or pre-invasive ductal carcinoma in situ (DCIS) disease, |
| | oncoplastic breast conserving surgery, and use of systemic therapy |
| b | Ultra-hypofractionated (26 Gy in five fractions) whole breast irradiation can be offered as (1) |
| | standard of care or (2) within a randomised controlled trial or prospective registration cohort |
| 2. | Chest wall irradiation |
| a | Moderate hypofractionation can be offered for chest wall irradiation without breast |
| | reconstruction |
| b | Moderate hypofractionation can be offered for chest wall irradiation regardless of time and type |
| | of breast reconstruction |
| c | Ultra-hypofractionation (26 Gy in five fractions) for chest wall irradiation without breast |
| | reconstruction can be offered as (1) standard of care or (2) within a randomised controlled trial |
| | or prospective registration cohort |
| d | Ultra-hypofractionation (26 Gy in five fractions) for chest wall irradiation after breast |
| | reconstruction can be offered within a randomised controlled trial or prospective registration |
| | cohort |
| 3. | Nodal irradiation |
| a | Moderate hypofractionation should be offered for nodal irradiation |
| b | Ultra-hypofractionation (26 Gy in five fractions) should not be offered for nodal irradiation until |
| | ongoing trials results are reported |
| 4. | Partial breast irradiation-patient selection for external beam radiotherapy |
| | Low risk-features suitable for partial breast irradiation are: luminal-like subtypes small tumour |
| | (≤3 cm), absence of lymph vascular space invasion, non-lobular invasive carcinoma, tumour |
| | grade 1-2, low-to-intermediate grade DCIS (sized ≤2.5 cm with clear surgical margins ≥3 mm), |
| | age at diagnosis 50 years or more, unicentric or unifocal lesion, clear surgical margins (>2 mm), |
| | node negative (including isolated tumour cells), and no use of primary systemic therapy and |
| | neoadjuvant chemotherapy |
| 5. | Partial breast irradiation-dose and fractionation |
| a | Moderate hypofractionation (40 Gy in 15 fractions) and ultra-hypofractionation (26-30 Gy in |
| | five fractions) represent acceptable schedules for external beam partial breast irradiation |
| b | Twice a day external beam partial breast irradiation dose and fractionations similar to those used |
| | in the RAPID trial should not be offered |

DCIS: ductal carcinoma in situ.

As used herein, the term “radiotherapy de-intensification” includes radiotherapy omission (i.e., not giving any radiotherapy at all) or simply reducing the radiation dose or the number of fractions of a given dose fractionation scheme.

The term “non-radiation therapy” denotes a therapy that is adequate for addressing or reducing the risk of invasive breast cancer in a subject, and that does not derive its therapeutic effect by radiation. Examples of such therapy include, chemo therapeutics, targeted and non-targeted, immune and non-immune modulated, monoclonal, other targeted and non-targeted, genomic therapies, antibody therapeutics, including, HER2 antibodies, including Trastuzumab. Often, in the present application, “non-radiation therapy” is denoted as “other therapy”.

As used herein, the term “local recurrence” denotes that a recurrence is in the operated breast. As used herein, local recurrence is interchangeable with ipsilateral breast tumor recurrence (IBTR).

As used herein, the term “regional recurrence” denotes that a recurrence is in regional lymph nodes (axillary, supraclavicular, infraclavicular, intrapectoral or internal mammary lymph nodes).

As used herein, the term “distant metastasis” refers to all other recurrences outside the above types of recurrences (local or regional). In other words, distant metastasis refers to recurrences in all other tissues of the body.

As used herein, “marker” and “biomarker” are used interchangeably and refer to a measured biological component such as a protein, an mRNA transcript, or a level of DNA amplification.

The term “control” refers to a sample or standard used for comparison with a sample which is being examined, processed, characterized, analyzed, etc. In some embodiments, the control is a sample obtained from a healthy patient or a non-tumor tissue sample obtained from a patient diagnosed with a breast tumor. In some embodiments, the control is a historical control or standard reference value or range of values (such as a previously tested control sample, such as a group of breast tumor patients with poor prognosis, or group of samples that represent baseline or normal values, such as the level of cancer-associated genes or proteins in non-tumor tissue).

The “Cox hazard ratio” is derived from the Cox proportional hazards model. Proportional hazards models are a class of survival models in statistics. Survival models relate the time that passes before some event occurs to one or more covariates that may be associated with that quantity of time. In the Cox proportional hazards model, the unique effect of a unit increase in a covariate is multiplicative with respect to the hazard rate. A “Cox hazard ratio” is the ratio of the hazard rates corresponding to the conditions described by two levels of an explanatory variable—a covariate, that is calculated using the cox proportional hazards model. The cox hazard ratio is the ratio of survival hazards for a one-unit change in the covariate. For example, the Cox hazard ratio may be the ratio of survival hazards for a 1 unit change in the logarithmic gene expression level. Thus, a larger value has a greater effect on survival or the hazard rate of the event being assessed, such as disease recurrence. In some embodiments, a hazard ratio (HR) greater than 1 indicates that an increased covariate level is associated with a worse patient outcome, where the covariate level is a marker expression level. In some embodiments, a HR less than 1 indicates that a decreased covariate level is associated with a better patient outcome, where the covariate level is a marker expression level.

As used herein, the term “non-tumor tissue sample” shall be taken to include any sample from or including a normal or healthy cell or tissue, or a data set produced using information from a normal or healthy cell or tissue. For example, the non-tumor sample may be selected from the group comprising or consisting of: (i) a sample comprising a non-tumor cell; (ii) a sample from a normal tissue; (iii) a sample from a healthy tissue; (iv) an extract of any one of (i) to (iii); (v) a data set comprising measurements of modified chromatin and/or gene expression for a healthy individual or a population of healthy individuals; (vi) a data set comprising measurements of modified chromatin and/or gene expression for a normal individual or a population of normal individuals; and (vii) a data set comprising measurements of the modified chromatin and/or gene expression from the subject being tested wherein the measurements are determined in a matched sample having normal cells. Preferably, the non-tumor sample is (i) or (ii) or (v) or (vii).

Detecting expression of a gene product denotes determining of a level of expression in either a qualitative or quantitative manner. Exemplary methods include, but are not limited to: microarray analysis, RT-PCR, Northern blot, in situ hybridization, Western blot, immunohistochemistry (IHC), fluorescent in situ hybridization (FISH), chromogenic in situ hybridization (CISH), multiplex immunofluorescence (MIF), enzyme-linked immunosorbent assay (ELISA), next generation sequencing (NGS), NGS-RNA seq, and mass spectrometry. For immunohistochemistry, immunofluorescence, and related technologies, the H-Score provides a scoring system that quantifies the abundance of a marker by capturing both the intensity and the proportion of the marker of interest from a captured image of the labeled sample.

As used herein, the term “multiplex immunofluorescence” (MIF), refers to a technique that simultaneously interrogates proteins within a single sample or tissue by using fluorescently labeled antibodies designed to be specific to the target of interest. In MIF, each target (specifically, the target's antigen) is recognized by a unique antibody having a unique fluorescent label. By using antibodies labeled with different fluorescent dyes, MI allows for the observation and analysis (including, but not limited to, spatial and volumetric analysis) of multiple antigens within the same biological sample at the same time.

The term “expression” denotes the process by which the coded information of a gene is converted into an operational, non-operational, or structural part of a cell, such as the synthesis of an RNA or protein. Gene expression can be influenced by external signals. For instance, exposure of a cell to a hormone may stimulate expression of a hormone-induced gene. Different types of cells can respond differently to an identical signal. Expression of a gene also can be regulated anywhere in the pathway from DNA to protein. Regulation can include controls on transcription, translation, RNA transport and processing, protein transport and processing, degradation of intermediary molecules such as mRNA, or through activation, inactivation, compartmentalization.

The expression of a nucleic acid molecule or protein in a sample can be altered relative to a control sample, such as a normal or non-tumor sample. Alterations in gene expression, such as differential expression, include but are not limited to: (1) overexpression; (2) underexpression; or (3) suppression of expression.

“Programmed death 1”, “PD-1”, “PD1”, “PDCD1” are used interchangeably herein and refers to the gene or gene product of PDCD1. In some embodiments, PD-1 is human PD-1. PD-1 is a cell surface receptor approximately 50 to 55 kDa. Structurally, PD-1 is composed of an extracellular domain that interacts with its ligands, PD-L1 and PD-L2, a transmembrane domain, and an intracellular domain containing an immunoreceptor tyrosine-based inhibitory motif (ITIM) and an immunoreceptor tyrosine-based switch motif (ITSM). In breast cancer, PD-1 and its ligands are overexpressed, which leads to suppression of the immune response against tumor cells, which enables tumor cells to proliferate and spread.

Interferon Regulatory Factor 9 (IRF9) is a member of the interferon regulatory factor family and is approximately 48 kDa. IRF9 contains a DNA-binding domain and acts a transcription factor as part of the ISGF3 complex that mediates type I interferon antiviral response by regulating the downstream expression of interferon-stimulated genes. In the context of breast cancer, IRF9 may be involved in pathways that contribute to tumor progression and resistance to therapy.[0078] Aldehyde dehydrogenase A1 (ALDHA1) is a member of the aldehyde dehydrogenase family of enzymes that play a crucial role in the oxidative metabolism of aldehydes into their corresponding carboxylic acids. ALDHA1 is a cytosolic enzyme that is pivotal in detoxifying aldehydes derived from alcohol metabolism and lipid peroxidation, thus contributing significantly to cellular homeostasis and protection against oxidative stress. Structurally, ALDHA1 exhibits a highly conserved active site that facilitates the catalytic conversion of aldehydes using NAD(P)⁺ as a cofactor, leading to the production of NADH (or NADPH) and the corresponding acid. High concentrations of this enzyme are found in the liver, where it is involved in the metabolism of ethanol, as well as in other organs contributing to the detoxification of endogenously produced and exogenously derived aldehydic compounds. Without being bound by theory, the upregulation of ALDHA1 is associated with breast cancer aggressiveness and poor prognosis due to its high expression in a tumor, which may provide a route for tumors to resist chemotherapy.

Glucose transporter 1 (GLUT1) is an integral membrane protein that facilitates the diffusion of glucose across the cellular membranes of mammalian cells. GLUT1 is characterized by its 12-transmembrane spanning domains with both of its N- and C-termini predicted to lie intracellularly. GLUT1 is encoded by the SLC2A1 gene is one of a family of 14 genes encoding GLUT proteins. GLUT1 is ubiquitously expressed in various tissues, with particularly high levels in erythrocytes and in the blood-brain barrier, where it ensures the efficient and continuous transport of glucose from the bloodstream into the brain and other cells. This protein is crucial for maintaining glucose homeostasis, enabling cells to uptake glucose in a concentration-dependent manner without the need for ATP. Without being bound by theory, elevated glucose levels in cancer cells is thought to be attributed to the upregulation of GLUT1, which may regulate signaling cascades involved in tumorigenesis of breast cancer.

When cancer is diagnosed, health care professionals attempt to ascertain the cancer's spread by a process called staging. A typical staging system used for breast cancer is the American Joint Committee of Cancer (AJCC) TNM system, effective January 2018. Understanding a Breast Cancer Diagnosis, ACS, Nov. 8, 2011, pp. 24-30. For staging under the TNM system, 7 pieces of information are used: the extent (size) of the tumor (T); the spread to nearby lymph nodes (N); the spread (metastasis) to distant sites (M); Estrogen Receptor (ER) status; Progesterone Receptor (PR) status; HER2 status; and grade of the cancer. When examining the primary tumor, T categories for breast cancer are used to denote the tumor's size, if it has spread to the skin or to the chest wall under the breast: TX (primary tumor cannot be assessed); TO (no evidence of a primary tumor); Tis (carcinoma in situ, e.g., DCTS or Paget disease of the breast with no associated tumor mass); T1 (includes T1a, T1b, and T1c), wherein the tumor is 2 cm (¾ of an inch) or less across; T2 (tumor is more than 2 cm but not more than 5 cm (2 inches) across); T3 (tumor is more than 5 cm across); T4 (includes T4a, T4b, T4c, and T4d), wherein the tumor is of any size growing into the chest wall or skin.

Part of the staging process also involves examining nearby lymph nodes (often through surgical removal and histopathological examination) to check for the presence of cancer cells. If cancer cells are not found in these lymph nodes, the patient is considered “node-negative” (often denoted as NO in staging terminology). This status generally indicates a lower stage of cancer, suggesting that the disease has not spread beyond the primary tumor site or to distant parts of the body. Consequently, node-negative patients may have a better prognosis and could be candidates for less aggressive treatment options compared to those with node-positive cancer, where cancer cells have been found in the lymph nodes, indicating a higher stage of disease.

### Methods

Methods of determining the risk of recurrence for cancer and treatment of the cancer, e.g., breast cancer, is provided. In some embodiments, a method 100 of determining a risk of recurrence for breast cancer includes obtaining 110 a sample provided by a subject as shown in FIG. 1. In some embodiments, the sample is characterized as estrogen receptor (ER) and/or progesterone receptor (PR) positive, and human epidermal growth factor 2 (HER2) negative. In some embodiments, the method includes assaying 120 the sample for markers, the markers including programmed cell death protein 1 (PD-1) and interferon regulatory factor 9 (IRF9). Assaying such markers provide a prognostic value in assessing the risk of recurrence for breast cancer, which is summarized in Table 3.

In some embodiments, the method includes assessing 130 the subject as having a significant risk of recurrence. In some embodiments, the method includes assessing the subject as having a significant risk of recurrence if the assaying step indicates elevated levels of expression of PD-1 and IRF9 in the sample. In some embodiments, the method includes recommending radiotherapy (RT) to the subject if the assaying step indicates the elevated levels of expression of PD-1 and IRF9 in the sample. In some embodiments, the method further includes recommending an RT boost to the subject if the assaying step indicates the jointly non-elevated levels of expression of PD-1 and IRF9 in the sample and additional risk factors are present including, but not limited to, lymphovascular invasion (LVI), young age, or high grade. In a non-limiting example, the subject having a significant risk of recurrence if the assaying step indicates jointly elevated levels of expression of PD-1 and IRF9 in the sample is represented in Table 3 for Class 5 that had breast conserving surgery (BCS) with or without radiotherapy. In some embodiments, the method includes assessing the subject as not having a significant risk of recurrence if the assaying step indicates levels of expression lower than the elevated levels of expression for the PD-1 and/or IRF9. In some embodiments, the method of determining a risk of recurrence for breast cancer includes assaying stromal PD-1 and/or IRF9 as shown in Table 3.

In some embodiments, the method includes delineating the sample into compartments, the compartments including, but not limited to, tumor, stroma, fat, and glass compartments. In some embodiments, the elevated levels of expression of PD-1 in the sample includes a number of cells that are positive for the PD-1 per surface area of the stroma region. In some embodiments, the number of cells that are positive for the PD-1 per surface area of the stroma compartment is >25 percentile of this biomarker population. In a non-limiting example, as shown for class 1 in Table 3, the sample from the subject where the biomarker is the number of cells that are positive for PD-1 expression per surface area of the stroma compartment and the marker is elevated when the marker is >25 percentile of this biomarker population and when elevated the subject treated with BCS without RT has a higher risk of recurrence of cancer, and may benefit from RT. In some embodiments, the PD-1 is quantitated as shown in Table 3. In some embodiments, the elevated levels of expression of IRF9 in the sample includes a percentage of cells that are positive for the IRF9 per surface area of the tumor compartment. In some embodiments, the percentage of cells that are positive for the IRF9 per surface area of the tumor compartment is >25 percentile of this biomarker population. In a non-limiting example, as shown for class 2 in Table 3, the sample from the subject where the biomarker is the intensity of cells that are positive for the IRF9 assessed by H-SCORE and elevated biomarker expression is >25 percentile of this biomarker population of non-TNBC, HER2 positive, without or without node positivity, samples that include various tumor grades, ages, and sizes, where elevated IRF9 biomarker expression does not suggest a significantly increased risk of recurrence and alone is not predictive for a benefit from RT. However, as shown in the non-limiting example for class 5 in Table 3, a sample from a subject that includes a biomarker algorithm combining the number of cells that are positive for the PD-1 per surface area of the stroma compartment which is >25 percentile of the biomarker population and the quantity of tumoral cell expression for IRF9 assessed by H-SCORE is >25 percentile of this biomarker population that had BCS with or without RT have an increased risk of recurrence of cancer and as shown in class 5, Table 3, is predicted for RT benefit.

In some embodiments, the markers include aldehyde dehydrogenase A1 (ALDHA1). In some embodiments, the method includes assessing the subject as having higher risk of recurrence if the assaying step indicates ahigh level of ALDHA1 in the tumor compartment, assessed by such means as H-SCORE, is >75 percentile of this biomarker population. In some embodiments, the method includes recommending RT to the subject if the assaying step indicates the elevated level of ALDHA1. In some embodiments, the method includes recommending a RT boost to the subject if the assaying step indicates the elevated level of ALDHA1 subject already underwent RT or is currently undergoing RT. In a non-limiting example, as shown for class 9 in Table 3, a high level of ALDHA1 expression in the tumor compartment of the sample that is >75 percentile of this biomarker population (various tumor grades, age, sample sizes, and the like), subjects that had BCS without RT had a higher risk of recurrence.

In some embodiments, the method includes assessing the subject as having increased risk of recurrence and a significant benefit to RT if the assaying step indicates an elevated level of ALDHA1 in the tumor compartment of the sample of >75 percentile of this biomarker population and the jointly elevated levels of expression of PD-1 and IRF9 in the sample. In a non-limiting example, as shown for class 10.1 in Table 3, these levels of ALDHA1, and not jointly elevated PD-1, and IRF9, do not indicate an increased risk of recurrence, and do not predict a significant benefit from RT.

In some embodiments, the markers include glucose transporter 1 (GLUT1). In some embodiments, the method includes assessing the subject as having increased risk of recurrence if the assaying step indicates a high level of GLUT1 in the tumor compartment of the sample, as assessed by a method such as H-SCORE, is >75 percentile of this biomarker population and there are jointly elevated levels of expression of PD-1 and IRF9 in the sample. In some embodiments, the method includes recommending de-intensification of the RT if the assaying step indicates a not high level of GLUT1 in the tumor compartment of the sample of >75 percentile of this biomarker population and not jointly elevated levels of expression of PD-1 and IRF9 in the sample. In a non-limiting example, as shown for Class 11 in Table 3, this high level of GLUT1 alone in the sample, where the patient is treated with BCS, without CT, and is HR+ve, not TNBC, not HER2+ve, not node positive, without RT, indicates higher risk of recurrence.

In some embodiments, the population includes levels of the markers from a plurality of samples from different individuals, the plurality of samples including multiple tumor grades, multiple individual ages, and multiple sizes of the samples.

In some embodiments, the markers include ER, PR, and HER2. In some embodiments, the assaying step includes determining the expression levels, or lack thereof, for the ER, PR, and HER2. In some embodiments, the determining of the expression levels, or lack thereof, for the ER, PR, and HER2 uses immunohistochemistry (IHC), fluorescent in situ hybridization (FISH), chromogenic in situ hybridization (CISH), enzyme-linked immunosorbent assay (ELISA), or the like. In some embodiments, the assaying step includes obtaining images, from at least a portion of the sample, of the markers. In some embodiments, the markers are indicated by light emission from excitation of immunofluorescent labels specific for each of the markers. In some embodiments, the assaying step includes counting the markers displayed on the obtained images. In some embodiments, the obtaining of the images step is performed using multiplex immunofluorescence (MIF). In some embodiments, the markers are interrogated together on the same one or more substantially planar slices of the sample. The results from the markers from MIF are normalized against the background signal from the sample. Because of this normalization, normalized data from other molecular and cellular labeling techniques (including, but not limited to, IHC, CISH, ELISA, and the like) may be combined with the normalized results from MIF. In some embodiments, the method includes using a non-linear model to analyze an interactivity between the markers. In some embodiments, the method includes using a non-linear model to analyze an interactivity between the markers as shown in Table 3.

In some embodiments, the subject is older than 50 years of age. In some embodiments, the subject is older than 55 years of age. In some embodiments, a status of the subject is node-negative. In some embodiments, a status of the subject is node-positive.

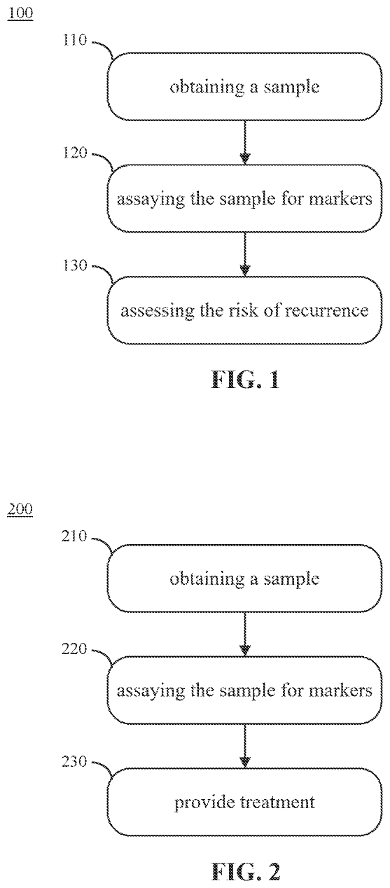

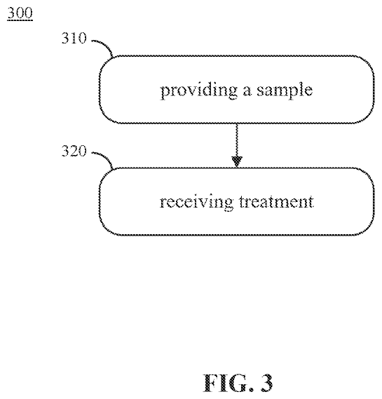

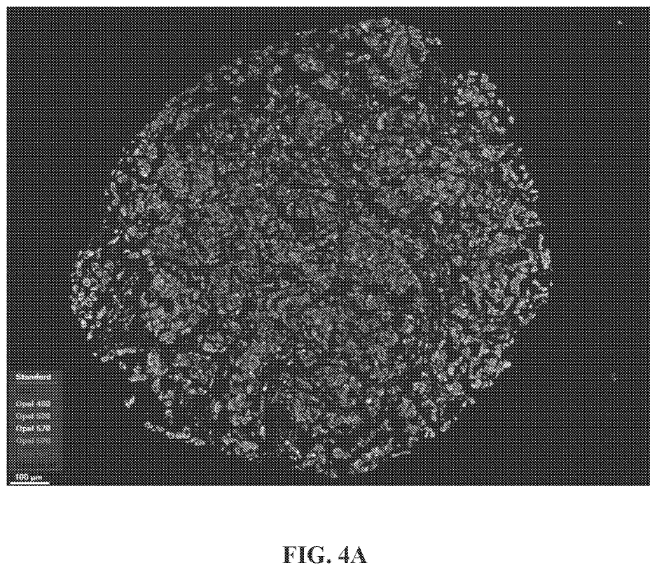

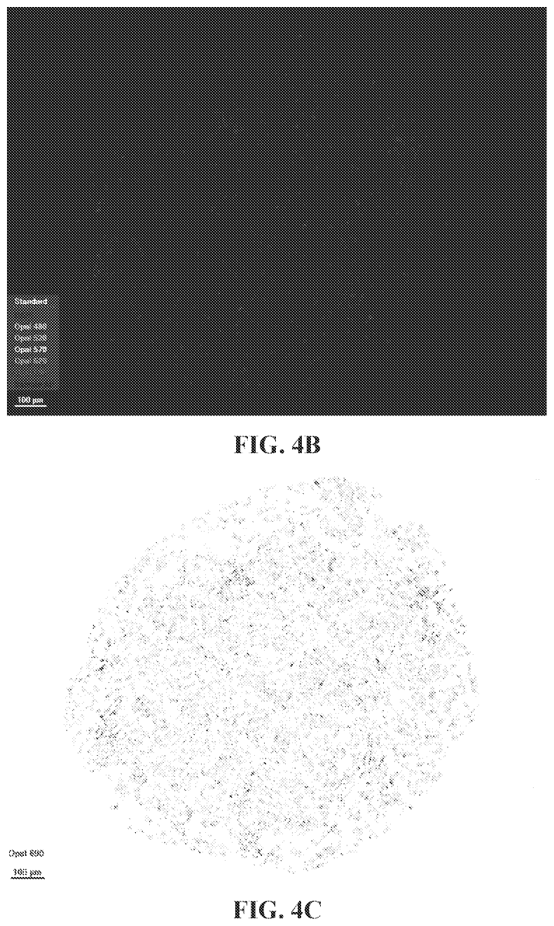

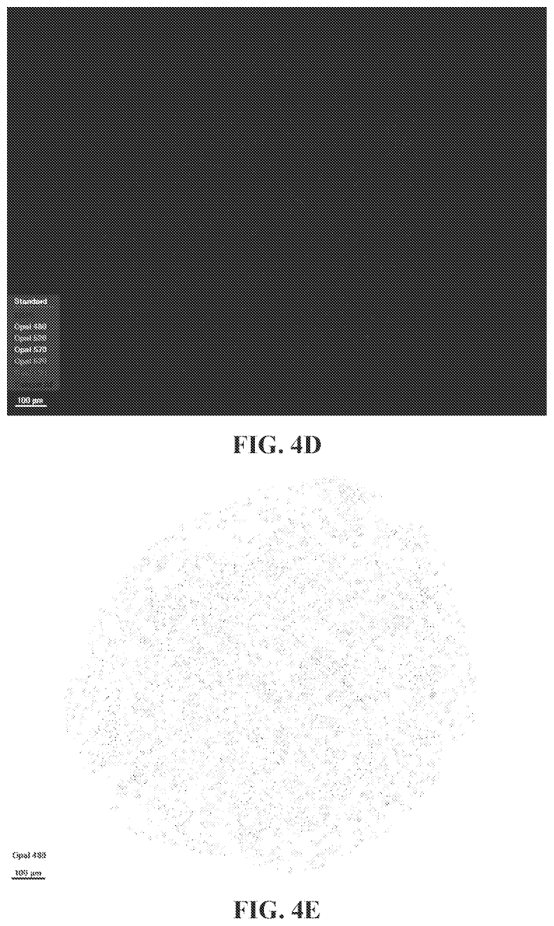

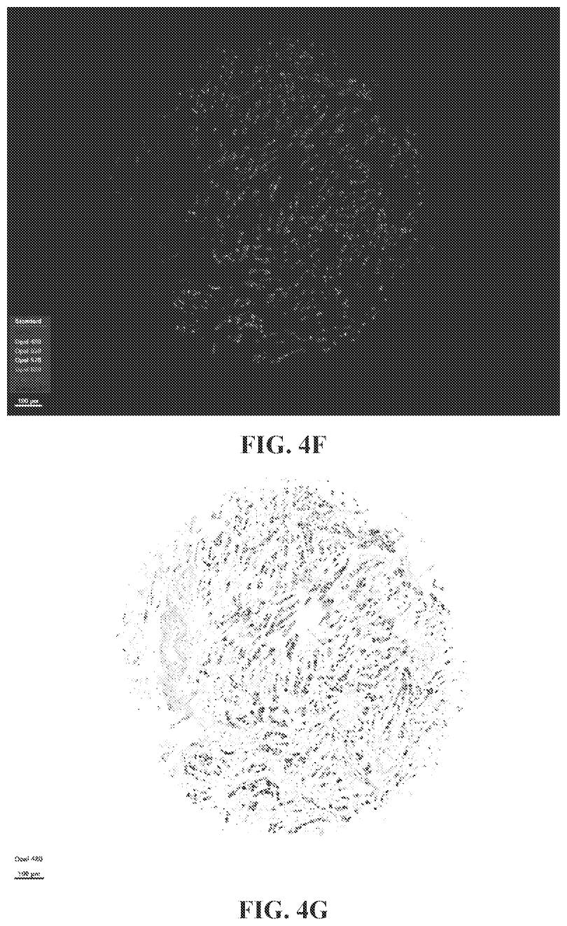

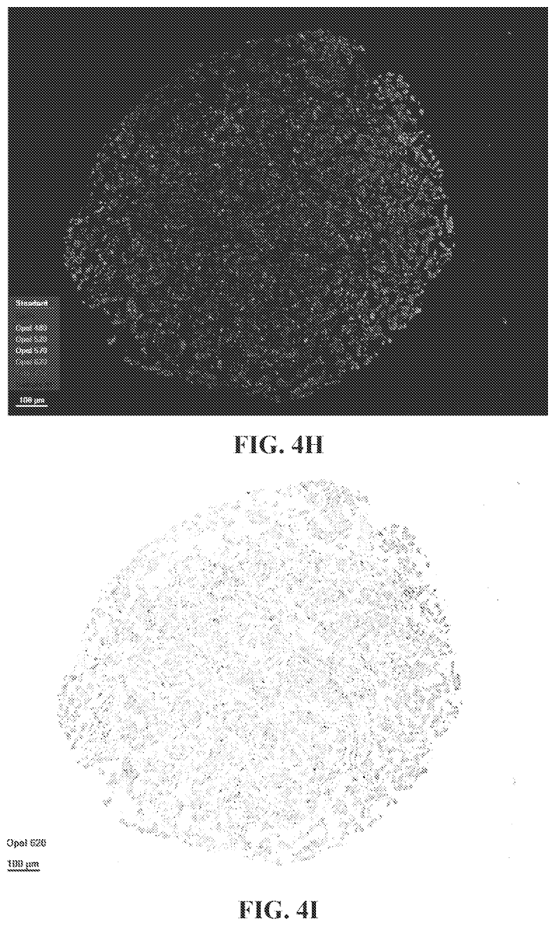

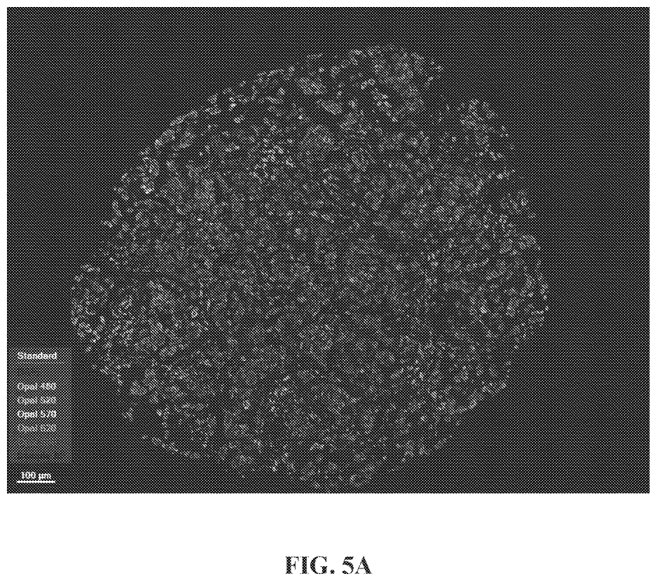

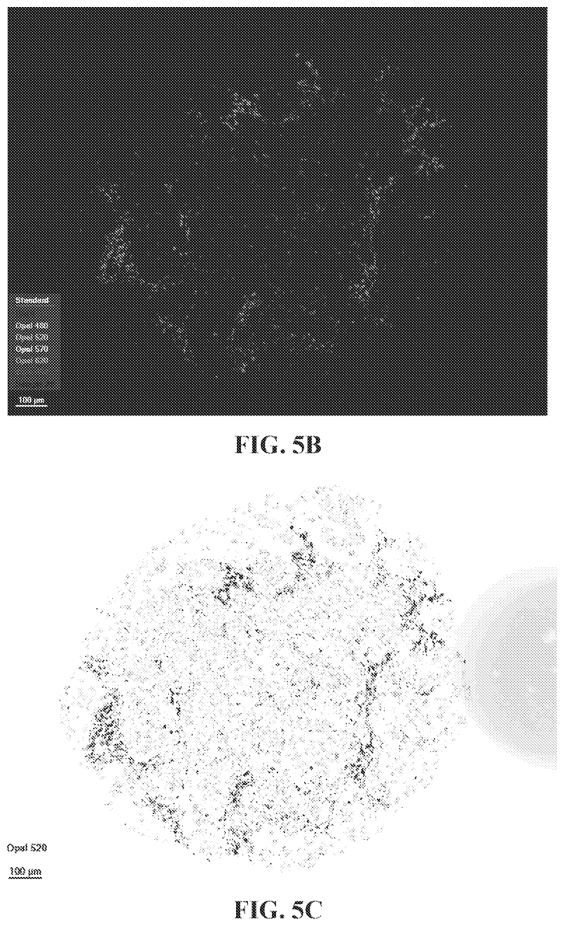

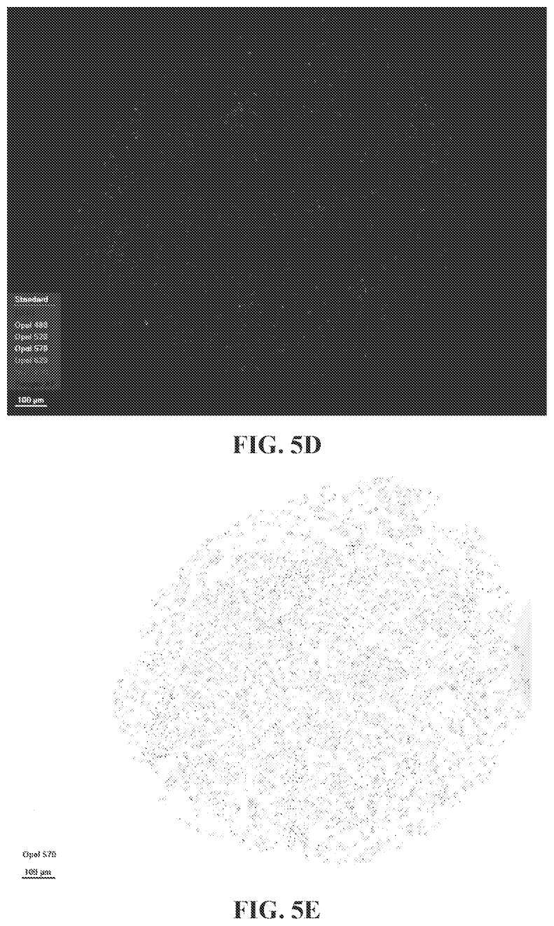

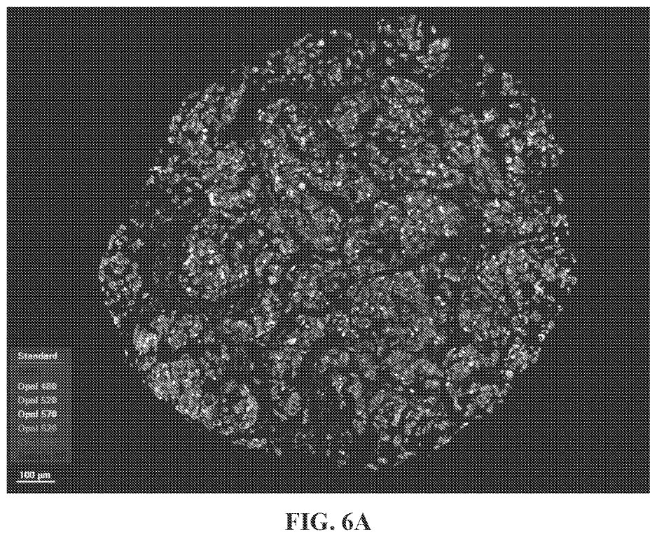

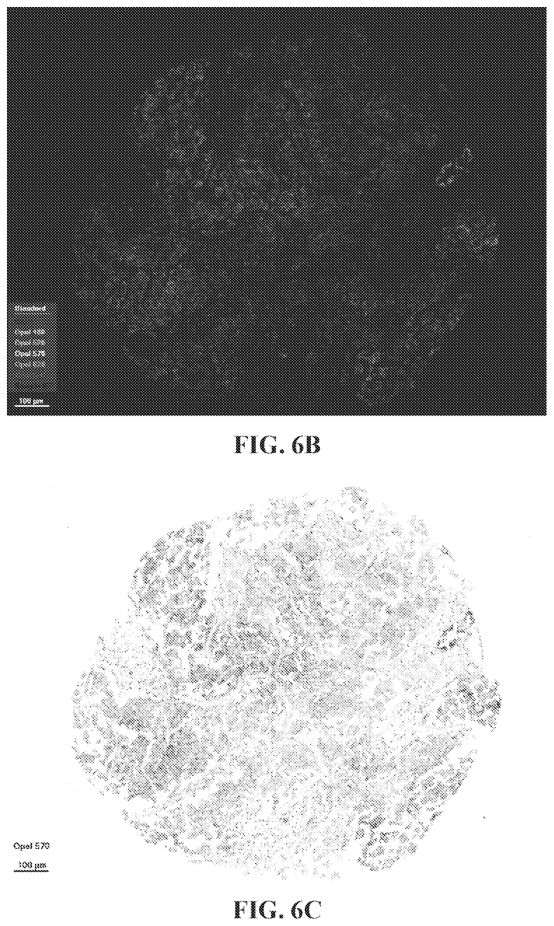

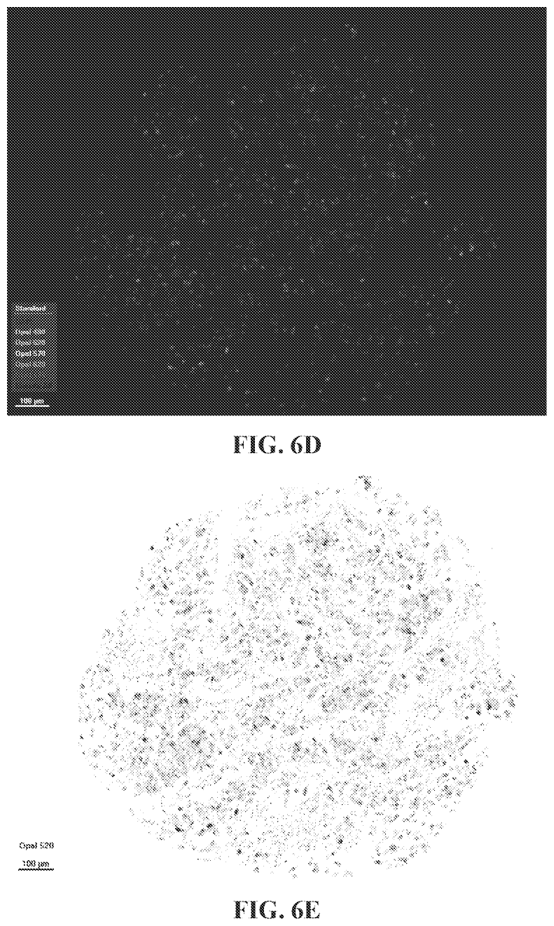

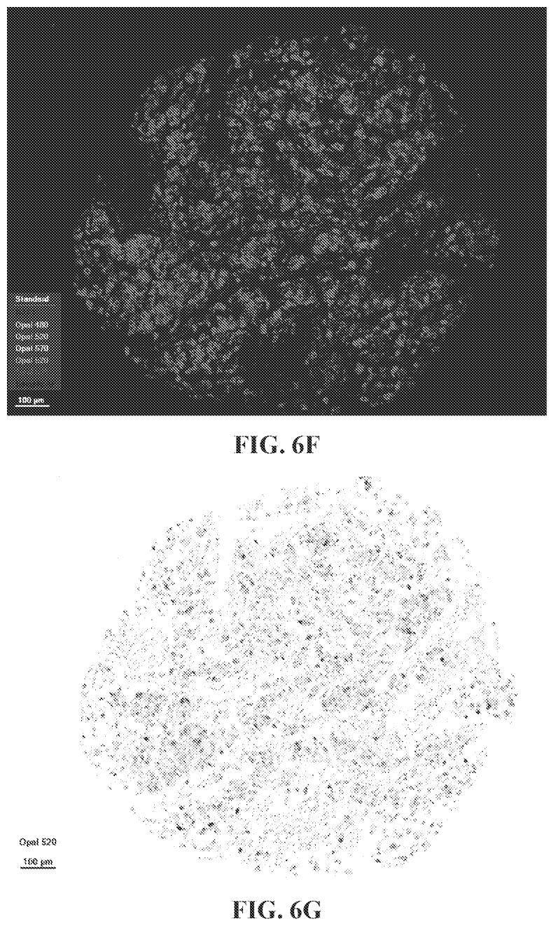

In some embodiments, a method 200 for treating cancer, e.g., breast cancer, is provided as shown in FIG. 2. The method 200 for treating the cancer includes obtaining 210 a sample provided by a subject, wherein the sample is characterized as ER and PR positive, and HER2 negative, assaying 220 the sample for markers, the markers including PD-1 and IRF9. In some embodiments, the method includes administering an RT boost to the subject if the assaying step indicates elevated levels of expression of PD-1 and IRF9 in the sample. A non-limiting example is shown for class 5 in Table 3. In some embodiments, the method of for treating cancer includes assaying stromal PD-1 and IRF9 as shown in Table 3.

In some embodiments, the method includes delineating the sample into compartments, the compartments including tumor, stroma, fat, and glass compartments. In some embodiments, the elevated levels of expression of PD-1 in the sample includes a number of cells that are positive for the PD-1 per surface area of the stroma region. In some embodiments, the number of cells that are positive for the PD-1 per surface area of the stroma compartment is >25 percentile of this biomarker population. In some embodiments, the elevated levels of expression of IRF9 in the sample includes a percentage multiplied by the intensity of expression of cells, such as an H-SCORE, that are positive for the IRF9 in the tumor compartment. In some embodiments, the PD-1 is quantitated as shown in Table 3. In some embodiments, the elevated expression of cells for the IRF9 marker in the tumor compartment is >25 of this biomarker population.

In some embodiments, the markers include ALDHA1. In some embodiments, the method includes treating the subject with the RT due to a higher risk of recurrence if the assaying step indicates a high level of ALDHA1 in the tumor compartment is >75 percentile of this biomarker population. In a non-limiting example, as shown for class 9 in Table 3, such an elevated level of ALDHA1 in the tumor compartment of the sample, where the subject undergoes BCS with or without RT, is predictive of an increased risk of recurrence of cancer and a greater benefit from RT. In some embodiments, the method includes de-intensifying the RT to the subject if the assaying step does not indicate a high level of ALDHA1 expression in the tumor compartment of the sample of >75 percentile of this biomarker population and the elevated levels of expression of PD-1 and IRF9 in the sample. In a non-limiting example, as shown for class 10 in Table 3, the high level of ALDHA1 and elevated level of IRF9 in the tumor compartment and PD-1 in the stroma compartment, where the subject undergoes BCS with or without RT, is predictive for an increased risk of recurrence but with a significant RT benefit, which would lead to a recommendation of RT.

In some embodiments, the markers include GLUT1. In some embodiments, the method includes de-intensifying the RT to the subject if the assaying step does not indicate a high level of GLUT1 in the tumor compartment of the sample of >75 percentile of this biomarker population and does not have jointly elevated levels of expression of PD-1 and IRF9 in the sample. A non-limiting example is shown for class 12 in Table 3. In some embodiments, the population include levels of the markers from a plurality of samples from different individuals, the plurality of samples including multiple tumor grades, multiple individual ages, and multiple sizes of the samples.

In some embodiments, the markers include ER, PR, and HER2. In some embodiments, the assaying step includes determining the expression levels, or lack thereof, for the ER, PR, and HER2. In some embodiments, the determining the expression levels, or lack thereof, for the ER, PR, and HER2 uses immunohistochemistry (IHC), fluorescent in situ hybridization (FISH), chromogenic in situ hybridization (CISH), enzyme-linked immunosorbent assay (ELISA), or the like. In some embodiments, the assaying step includes obtaining images, from at least a portion of the sample, of the markers. In some embodiments, the markers are indicated by light emission from excitation of immunofluorescent labels specific for each of the markers. In some embodiments, the obtaining of the images step is performed using multiplex immunofluorescence (MIF). In some embodiments, the markers are interrogated together on the same one or more substantially planar slices of the sample. In some embodiments, the assaying step includes counting the markers displayed on the obtained images. In some embodiments, a trained healthcare professional, e.g., pathologist, indicates the tumor, stroma, fat, and glass compartments on the obtained images of the sample. Because each obtained image represents an optical slice of the sample, each obtained image needs to be evaluated and delineated into the appropriate one or more compartments where applicable.

In some embodiments, the method includes creating a training set including the category masks, the obtained images, and the expression levels. In some embodiments, the method includes training a non-linear model using the training set to output a score, the score indicating whether the subject will be responsive to RT in reducing a risk to breast cancer recurrence. In some embodiments, the method includes using a non-linear model to analyze an interactivity between the markers. In some embodiments, the method includes using a non-linear model to analyze an interactivity between the markers as shown in Table 3.

In some embodiments, the subject is older than 50 years of age. In some embodiments, the subject is older than 55 years of age. In some embodiments, a status of the subject is node-negative. In some embodiments, a status of the subject is node-positive.

In some embodiments, a method 300 for treating breast cancer is provided, the method including providing 310 a sample, wherein the sample is characterized as ER and PR positive, and HER2 negative; receiving 320 an RT boost or escalating an RT if the sample includes jointly elevated levels of expression of PD-1 and IRF9; and de-escalating the RT if the sample does not include jointly elevated levels of expression for the PD-1 and IRF9.

In some embodiments, the sample is delineated into compartments, the compartments including tumor, stroma, fat, and glass compartments. In some embodiments, the elevated levels of expression of PD-1 in the sample includes a number of cells that are positive for the PD-1 per surface area of the stroma region. In some embodiments, the number of cells that are positive for the PD-1 per surface area of the stroma compartment is >25 percentile of this biomarker population. In some embodiments, the elevated levels of expression of IRF9 in the sample includes a percentage of cells that are positive for the IRF9 times their intensity, evaluated by a method such as H-SCORE, for cells in the tumor compartment. In some embodiments, the intensity multiplied by the percentage of cells, such as an H-SCORE, that are positive for the IRF9 in the tumor compartment is >25 percentile of this biomarker population. In some embodiments, the population includes levels of the markers from a plurality of samples from different individuals, the plurality of samples including multiple tumor grades, multiple individual ages, and multiple sizes of the samples.

In some embodiments, the markers include ALDHA1. In some embodiments, the method includes receiving the RT due to a higher risk of recurrence if the sample includes a high level of ALDHA1 in the tumor compartment is >75 percentile of this biomarker population. In some embodiments, the method includes receiving an RT boost if already had the RT or is currently undergoing the RT. In some embodiments, the method includes de-intensifying the RT if the sample does not include a high level of ALDHA1 expression in the tumor compartment of >75 percentile of this biomarker population and the elevated levels of expression of PD-1 and IRF9.

In some embodiments, the markers include glucose transporter 1 (GLUT1). In some embodiments, the method includes de-intensifying the RT if the sample does not include a high level of GLUT1 in the tumor compartment of the sample of >75 percentile of this biomarker population and does not include elevated levels of expression of PD-1 and IRF9.

In some embodiments, a method of determining a risk of recurrence for breast cancer is provided, the method including obtaining a sample provided by a subject, wherein the sample is characterized as ER and/or PR positive, and not HER2 positive; assaying the sample for markers, the markers comprising PD-1 and IRF9; and assessing the subject as having a significant risk of recurrence if the assaying step indicates jointly elevated levels of expression of PD-1 and IRF9 in the sample; assessing the subject as having significantly decreased risk of recurrence if the assaying step indicates levels of expression lower than the elevated levels of expression for the PD-1 and IRF9.

In some embodiments, a method for treating breast cancer is provided, the method including obtaining a sample provided by a subject, wherein the sample is characterized as ER and/or PR positive, and not HER2 enhanced; assaying the sample for markers, the markers comprising PD-1 and IRF9; and providing an RT boost to the subject if the assaying step does not indicate jointly elevated levels of expression of PD-1 and IRF9 in the sample and the subject has other risk factors including, but not limited to, lymphovascular invasion (LVI), young age, or high grade; and de-escalating the RT of the subject if the assaying step indicates levels of expression lower than the elevated levels of expression for the PD-1 and IRF9 and the subject does not have other risk factors, which include, but are not limited to, lymphovascular invasion (LVI), young age, or high grade.

In some embodiments, a method for treating breast cancer is provided, the method including providing a sample, wherein the sample is characterized as ER and/or PR positive, and not HER2 enhanced; receiving an RT boost if the sample does not include jointly elevated levels of expression of PD-1 and IRF9 and has the additional risk factors present including, but not limited to, lymphovascular invasion (LVI), young age, or high grade; and de-escalating an RT if the sample includes levels of expression of PD-1 and IRF9 that are lower than the elevated levels of expression for the PD-1 and IRF9 and does not have the additional risk factors present including, but not limited to, lymphovascular invasion (LVI), young age, or high

In some embodiments, the method can include conducting any one or more of the steps in a single row shown in Table 3. In the methods related to diagnostics, the methods can include using, for every row, the columns designated as Biomarker or Biomarker Algorithm, Method of Quantitation, Population (group), Finding, Prognostic for recurrence (in the context of a medical recommendation), and RT Treatment Benefit (in the context of a medical recommendation). In the methods related to treatments, the methods can include using, for every row, the columns designated as Class, Biomarker or Biomarker Algorithm, Method of Quantitation, Population, Finding, Prognostic for recurrence, and RT Treatment Benefit.

In a non-limiting example, as illustrated for Class 1 in Table 3, PD-1 is interrogated, where the PD-1 expression level is quantitated as the number of cells with PD1 per surface area in the stromal compartment of the sample section. This expression level of PD-1 is compared against the PD-1 expression level of the group described in the “Population (group)” column. Here, the group includes tumor samples from patients that were treated with BCS, no CT, are HR+ve, not TNBC, not HER2 enhanced, not node positive, with or without RT, and biomarker level of the Group is known. Accordingly, the group is varied in grade of the tumor, age of the subject, and tumor size. If the ratio of the number of cells expressing PD-1/tumor surface area as determined by MIF is >25 percentile of this ratio of the biomarker stromal PD-1 expression/stromal surface area from the population (group) samples, then the subject is recommended or administered BCS with RT. However, such a treatment for this class is associated with a higher risk of recurrence of the cancer. As non-limiting example, the elevated biomarker expression is the ratio of stromal PD1 positive cell count divided by stromal surface area that is greater than the >25% of this biomarker population (see Table 4D). Low expression of this biomarker is not elevated expression of this biomarker.

For Class 1.1 in Table 3 examining the PD-1 using a univariable analysis in a subset of patients with elevated PD-1 levels of >25 percentile than this biomarker population in Group (here with elevated PD-1 expression), patients benefit from RT after BCS. Therefore, patients with elevated biomarker expression benefitted from RT. As shown in Class 1.2 in Table 3, the subset of patients with non-elevated levels of PD-1 expression did not benefit from RT after BCS.

In a non-limiting example, as illustrated for class 2 in Table 3, IRF9 is interrogated using MIF, whereby the IRF9 expression level is quantitated as the cellular expression of IRF9 by percentage in the tumor compartment of the sample section. Alternatively, IRF9 can be quantitated as the amount of the IRF9 protein present in the tumor by H-SCORE, as in class 2. This expression level of IRF9 is compared against IRF9 expression level of the group described in the “Population (group)” column. Here, the group includes tumor samples that are Hormone Receptor (HR) positive (therefore, not TNBC) and not HER2 enhanced. Accordingly, the group is varied in grade of the tumor, age of the sample donor, and tumor size. If the ratio of the number of cells expressing PD-1/tumor surface area as determined by MIF is >25 percentile of the ratio of the biomarker population of the stromal PD-1 expression/stromal surface area from the population (group) samples and the IRF9 expression level determined by MIF is >25 percentile than this biomarker population of IRF9 expression level from the group samples, then the subject is recommended or administered RT, as in class 5. However, the class in which IRF9 expression level alone (class 2) determined by MIF is >25 percentile than this biomarker population of IRF9 expression level from the group samples is not associated with a significantly increased risk of recurrence of the cancer and there is no added benefit of RT for class 2.

In a non-limiting example, as illustrated for Class 3 in Table 3, Class 1 (PD-1) and/or Class 2 (IRF9) markers are interrogated, where the IRF9 and PD-1 expression levels are quantitated under a linear model that combines the class 1 marker with the class 2 marker, e.g., tumoral IRF9>25 percentile of this biomarker population and/or stromal PD1>25 percentile of this biomarker population as defined from Classes 1 and 2. For IRF9 and PD-1, their respective expression levels are quantitated as described in Classes 1 and 2 of Table 3. The expression levels of IRF9 and PD-1 are compared against the IRF9 and PD-1 expression levels of the “Population (group)” column. Here, the group includes tumor samples from patients treated with BCS, without CT, with or without RT, are HR+ve, not TNBC and not HER2 positive, not node positive, the expression level of both biomarkers in the group are known. Accordingly, the group is varied in grade of the tumor, age of the sample donor, and tumor size. If the IRF9 and PD-1 expression levels determined by MIF and are assessed in a an algorithm combining the two biomarkers in a simply linear additive manner with elevated levels >25 percentile of these two biomarker populations of the IRF9 and PD-1 expression levels from the group samples, then there is not a significant association with the biomarkers and recurrence risk and the subject cannot be recommended or administered BCS with or without RT based on the biomarkers combined in a simple linear additive manner.

In a non-limiting example, as illustrated for Class 4 in Table 3, Class 1 (PD-1) and Class 2 (IRF9) markers are interrogated, where the PD-1 and IRF9 expression levels are quantitated by combining the class 1 marker and the class 2 marker, e.g., stromal PD1>25 percentile of this biomarker population and tumoral IRF9>25 percentile of this biomarker population as defined from Classes 1 and 2 respectively. For PD-1 and IRF9, their respective expression levels are quantitated as described in Classes 1 and 2 of Table 3. The expression levels of IRF9 and PD-1 are compared against the IRF9 and PD-1 expression levels of the “Population (group)” column. Here, the Group includes tumor samples from patients treated with BCS, without CT, with or without RT, are HR+ve, not TNBC and not HER2 positive, not node positive, the expression level of both biomarkers in the Group are known and tumoral IRF9 expression was low <25 percentile of this biomarker population. Accordingly, the Group is varied in grade of the tumor, age of the sample donor, and tumor size. Here, elevated expression vs low expression of biomarker is not prognostic for recurrence risk after BCS and not predictive for RT benefit. Accordingly, the prognostic utility of the combination of stromal PD1 marker and IRF9 marker is limited due to the lack of statistical significance between the two markers in a subset of patients with lower IRF9 expression, as analyzed in Class 4.

In a non-limiting example, as illustrated for Class 4.1 in Table 3, Class 1 (PD-1) and Class 2 (IRF9) markers are interrogated, where the PD-1 and IRF9 expression levels are quantitated by combining the class 1 marker and the class 2 marker, e.g., stromal PD1>25 percentile of this biomarker population and tumoral IRF9<50 percentile of this biomarker population as defined from Classes 1 and 2 respectively. For PD-1 and IRF9, their respective expression levels are quantitated as described in Classes 1 and 2 of Table 3. The expression levels of IRF9 and PD-1 are compared against the IRF9 and PD-1 expression levels of the “Population (group)” column. Here, the group includes tumor samples from patients treated with BCS, without CT, with or without RT, are HR+ve, not TNBC and not HER2 positive, not node positive, the expression level of both biomarkers in the group are known and tumoral IRF9 expression was not high (<50 percentile of this biomarker population). Accordingly, the group is varied in grade of the tumor, age of the sample donor, and tumor size. Here, elevated expression vs low expression of biomarker is not prognostic for recurrence risk after BCS and not predictive for RT benefit. Accordingly, the prognostic utility of the combination of stromal PD1 marker and IRF9 marker is limited due to the lack of statistical significance between the two markers in a subset of patients with a not high IRF9 level of expression, as analyzed in Class 4.1.

In a non-limiting example, as illustrated for Class 4.2 in Table 3, Class 1 (PD-1) and Class 2 (IRF9) markers are interrogated, where the PD-1 and IRF9 expression levels are quantitated by combining the class 1 marker and the class 2 marker, e.g., stromal PD1>25 percentile of this biomarker population as defined from Class 1. For PD-1, its respective expression level is quantitated as described in Class 1 of Table 3. The expression levels of IRF9 and PD-1 are compared against the IRF9 and PD-1 expression levels of the “Population (group)” column. Here, the group includes tumor samples from patients treated with BCS, without CT, with or without RT, are HR+ve, not TNBC and not HER2 positive, not node positive, the expression level of both biomarkers in the group are known and tumoral IRF9 expression was elevated (>25 percentile of this biomarker population). The group is varied in grade of the tumor, age of the sample donor, and tumor size. Here, elevated expression vs low expression of biomarker is not prognostic for recurrence risk after BCS and not predictive for RT benefit. Accordingly, the prognostic utility of the combination of stromal PD1 marker and IRF9 marker is limited due to the lack of statistical significance between the two markers in a subset of patients with elevated IRF9 expression level, as analyzed in Class 4.2.

In a non-limiting example, as illustrated for Class 5 in Table 3, Class 1 (PD-1) and Class 2 (IRF9) markers are interrogated, where the PD-1 and IRF9 expression levels are quantitated by a non-linear integration of the class 1 marker and the class 2 marker, e.g., PD1 expression/stromal tumor area (>25 percentile of this biomarker population) and elevated IRF9 expression quantity (>25 percentile of this biomarker population). For PD-1 and IRF9, their respective expression levels are quantitated as described in Classes 1 and 2 of Table 3. The expression levels of IRF9 and PD-1 are compared against the IRF9 and PD-1 expression levels of the “Population (group)” column. Here, the group is the same as described in group 3. Here, Non-linear joint (PD1 and IRF9) term elevated expression was prognostic for recurrence after BCS and predictive for RT benefit. Accordingly, the prognostic utility of the combination of stromal PD1 marker and IRF9 marker from the non-linear integration of their respective expressive levels is statistically significance between the two markers with respect to determining the risk of recurrence after BCS and being predictive with respect to RT.

In a non-limiting example, as illustrated for Class 6 in Table 3, Class 1 (PD-1) and Class 2 (IRF9) markers are interrogated, where the PD-1 and IRF9 expression levels are quantitated in the same manner as Class 5. Using MIF, PD1 was quantitated as the percent of cells positive for PD1 expression in the stromal compartment, and the method of Class 2. The group is the same as Class 5. Here, non-linear joint (PD1 and IRF9) term elevated expression was not prognostic for recurrence after BCS and not predictive for RT benefit. These results demonstrate how the method of quantifying the PD1 expression in the stromal compartment is critical to the observed outcome.

In a non-limiting example, as illustrated for Class 7 in Table 3, Class 1 (PD-1) and Class 2 (IRF9) markers are interrogated, where the PD-1 and IRF9 expression levels are quantitated in the same manner as Class 5. Using the quantitation of Class 1, MIF, and the percent of cells in the tumor compartment that were positive for IRF9 expression, PD1 was quantitated as the percent of cells positive for PD1 expression in the stromal compartment, and the method of Class 2. The group is the same as Class 5. Here, the non-linear joint (PD1 and IRF9) term elevated expression was prognostic for recurrence after BCS and predictive for RT benefit.

In a non-limiting example, as illustrated for Class 9 in Table 3, tumoral ALDHA1 is interrogated using MIF and quantitating the amount of expression of ALDHA1 in the tumor compartment cells (e.g., % times intensity, or H-SCORE). A high biomarker expression is defined as quantity of ALDHA1 expression in tumor cells, such as H-SCORE, for >75 percentile of this biomarker population. The expression level of ALDHA1 is compared against the ALDHA1 expression levels of the “Population (group)” column. Here, the group includes tumor samples from patients treated with BCS, without CT, with or without RT, are HR+ve, not TNBC and not HER2 positive, not node positive, the expression level of both biomarkers in the group are known and tumoral IRF9 expression was low 525 percentile of this biomarker population. Accordingly, the group is varied in grade of the tumor, age of the sample donor, and tumor size. That is, high vs. not high tumoral ALDHA1 expression was significantly associated with increased risk of recurrence.

In a non-limiting example, as illustrated for Class 10 in Table 3, Class 1 (PD-1), Class 2 (IRF9), and Class 9 (ALDHA1) markers are interrogated, where PD-1, IRF9, ALDHA1 expression levels are quantitated as described in Classes 1, 2, and 9 respectively in non-linear model, where tumor ALDHA1 was high at >75 percentile of this biomarker population of the group, stromal PD-1 is >25 percentile of this biomarker population of the group, and tumoral IRF9 is >25 percentile of this biomarker population of the group. The expression levels of ALHDA1, IRF9, and PD-1 are compared against the expression levels in the “Population (group)” column where the group expression levels of PD-1 and TRF9 are known. Here, the group includes tumor samples from patients treated with BCS, without CT, with or without RT, are HR+ve, not TNBC and not HER2 positive, not node positive. Accordingly, the group is varied in grade of the tumor, age of the sample donor, and tumor size. The ALDHA1 biomarker high expression was prognostic for increased recurrence rate and predictive for RT benefit in population only when non-linear joint (PD1 and IRF9) term had elevated expression, but otherwise was not. Lastly, there was statistically significance in joint analysis in the group population when ALDHA1 expression is high, stromal PD1 positive cells/stromal area is elevated, and tumoral IRF9 expression is elevated (see also Class 1, 2, and 9).

In a non-limiting example, as illustrated for Class 10.1 in Table 3, Class 1 (PD-1), Class 2 (IRF9), and Class 9 (ALDHA1) markers are interrogated, where PD-1, IRF9, ALDHA1 expression levels are quantitated as described in Classes 1, 2, and 9 respectively in non-linear model. The expression levels of ALHDA1, IRF9, and PD-1 are compared against the expression levels in the “Population (group)” column where the group expression levels of PD-1 and TRF9 are known. Here, the group includes tumor samples from patients treated with BCS, without CT, with or without RT, are HR+ve, not TNBC and not HER2 positive, not node positive. Accordingly, the group is varied in grade of the tumor, age of the sample donor, and tumor size. Tumor ALDHA1 (Class 9) was high at >75 percentile of this biomarker population of the group, stromal PD-1 (Class 1) is >25 percentile of this biomarker population of the group, and tumoral IRF9 (Class 2) is >25 percentile of this biomarker population of the group. The ALDHA1 marker high expression was prognostic for increased recurrence rate and predictive for RT benefit in population only when non-linear joint (PD1 and IRF9) term had elevated expression, but otherwise was not. Lastly, there was statistically significance in joint analysis in the group population when ALDHA1 expression is high, stromal PD1 positive cells/stromal area is elevated, and tumoral IRF9 expression is elevated (see also Class 1, 2, and 9).

In a non-limiting example, as illustrated for Class 10.2 in Table 3, Class 1 (PD-1), Class 2 (IRF9), and Class 9 (ALDHA1) markers are interrogated, where PD-1, IRF9, ALDHA1 expression levels are quantitated as described in Classes 1, 2, and 9 respectively in non-linear model. The expression levels of ALHDA1, IRF9, and PD-1 are compared against the expression levels in the “Population (group)” column where the group expression levels of PD-1 and TRF9 are known. Here, the group includes tumor samples from patients treated with BCS, without CT, with or without RT, are HR+ve, not TNBC and not HER2 positive, not node positive. Accordingly, the group is varied in grade of the tumor, age of the sample donor, and tumor size. Tumor ALDHA1 (Class 9) was high at >75 percentile of this biomarker population of the group, stromal PD-1 (Class 1) is >25 percentile of this biomarker population of the group, and tumoral IRF9 (Class 2) is >25 percentile of this biomarker population of the group. The ALDHA1 marker high expression was not prognostic for increased recurrence rate when non-linear joint (PD1 and IRF9) term did not have elevated expression. Lastly, there was not statistically significant results in the joint analysis of the group population where ALDHA1 expression is high and either stromal PD1 positive cells/stromal area or tumoral IRF9 expression is not elevated.

In a non-limiting example, as illustrated for Class 11 in Table 3, the GLUT marker was interrogated using MIF and quantitated the amount of expression of GLUT1 in the tumor compartment cells (e.g., % times intensity, or H-SCORE). The expression levels of tumoral GLUT1 is compared against the expression levels in the “Population (group)” column where the group expression level of the GLUT1 marker therein are known. Here, the group includes tumor samples from patients treated with BCS, without CT, with or without RT, are HR+ve, not TNBC and not HER2 positive, not node positive. Accordingly, the group is varied in grade of the tumor, age of the sample donor, and tumor size. The GLUT1 marker high expression was was prognostic for increased recurrence rate in the population. That is, high vs not high tumoral GLUT1 expression was significantly associated with increased risk of recurrence.

In a non-limiting example, as illustrated for Class 12 in Table 3, Class 1 (PD-1) and Class 2 (IRF9), Class 11 (GLUT1) markers are interrogated in a manner similar to each respective Class quantitation. The marker thresholds are tumor GLU1>75 percentile of this biomarker population of the group, stromal PD-1>25 percentile of this biomarker population of the group, and tumoral IRF9>25 percentile of this biomarker population of the group. Here, the group includes tumor samples from patients treated with BCS, without CT, with or without RT, are HR+ve, not TNBC and not HER2 positive, not node positive, the expression level of both PD-1 and IRF9 markers in the group are known. Accordingly, the group is varied in grade of the tumor, age of the sample donor, and tumor size. Here, both not-high expression of GLUT1 biomarker and non-elevated joint (PD1 and IRF9) term expression was prognostic for decreased recurrence rate and predictive for diminished RT benefit in population. There was statistical significance for both the prognostic for recurrence and RT when both GLUT1 expression is not high and (either stromal PD1 positive cells/stromal area or tumoral IRF9 expression is not elevated).

In a non-limiting example, as illustrated for Class 13 in Table 3, Class 1 (PD-1) and Class 2 (IRF9), Class 9 (ALDHA1), Class 11 (GLUT1) markers are interrogated in a manner similar to each respective Class quantitation. However, these markers are combined in a non-linear model for analysis. The marker thresholds are tumor GLU1>75 percentile of this biomarker population of the group, ALDHA1 75 percentile of this biomarker population of the group, stromal PD-1>25 percentile of this biomarker population of the group, and tumoral IRF9>25 percentile of this biomarker population of the group. Here, the group includes tumor samples from patients treated with BCS, without CT, with or without RT, are HR+ve, not TNBC and not HER2 positive, not node positive, the expression levels of PD-1, IRF9, GLUT1, and ALDHA1 markers in the group are known. Accordingly, the group is varied in grade of the tumor, age of the sample donor, and tumor size. Here, joint non-linear term of not high expression of ALDHA1 and not high expression of GLUT1 and not elevated expression Non-linear joint (PD1 and IRF9) term was prognostic for decreased recurrence risk and predictive of diminished RT benefit. There was statistical significance for both the prognostic for recurrence and RT when both GLUT1 expression is not high and (either stromal PD1 positive cells/stromal area or tumoral IRF9 expression is not elevated).

TABLE 3

| Class/ | Biomarker or | | Patient | | | |
|---|---|---|---|---|---|---|
| Ref | biomarker | Method of | population | | Prognostic for | RT treatment |
| Table | algorithm | quantitation | (group) | Finding | recurrence | benefit |
| Class | Biomarker; | The number of | Patients treated | In the patient | Not | Not |
| 1/ | Stromal PD1 | cells in the | with BCS | population, | statistically | statistically |
| Table | positive | stromal | without | elevated | significant in | significant in |
| 4A | cells/stomal | compartment | chemotherapy | expression vs low | joint analysis | joint analysis |
| | surface area; | with PD1 | (CT), and | expression of | in patient | in patient |
| | Elevated | expression | HR +ve, not | biomarker is not | population | population |
| | biomarker | divided by the | TNBC, not | prognostic for | | |
| | expression is | surface area of | HER2 +ve, not | recurrence risk | | |
| | defined as the | the stromal | node positive | after BCS and is | | |
| | ratio of stromal | compartment | and | not predictive for | | |
| | PD1 positive | | biomarker had | RT benefit | | |
| | cell count | | known | | | |
| | divided by | | expression | | | |
| | stromal surface | | with or without | | | |
| | area that is | | Radiotherapy | | | |
| | greater than | | | | | |
| | the >25 percentile | | | | | |
| | of patient | | | | | |
| | population (see | | | | | |
| | Table 4D). Low | | | | | |
| | expression is not | | | | | |
| | elevated | | | | | |
| | expression. | | | | | |
| Class | Biomarker: | Same as 1 | Elevated | NA | In univariable | Patients with |
| 1.1/ | Same as table 3, | | biomarker | | analysis, in | elevated |
| Table | class 1 | | expression, | | subset of | biomarker |
| 4B | | | AND | | patients with | expression |
| | | | patients treated | | elevated | benefited |
| | | | with BCS | | biomarker | from RT |
| | | | without CT, | | expression, | |
| | | | AND | | patients | |
| | | | HR +ve, not | | benefited from | |
| | | | TNBC, not | | RT after BCS | |
| | | | HER2 +ve | | | |
| | | | with or without | | | |
| | | | Radiotherapy | | | |
| Class | Biomarker: | Same as 1 | Not elevated | NA | In univariable | Patients |
| 1.2/ | Same as table 3, | | biomarker | | analysis, in | without |
| Table | class 1 | | expression, | | subset of | elevated |
| 4C | | | AND | | patients with | biomarker |
| | | | patients treated | | non- elevated | expression |
| | | | with BCS | | biomarker | did not |
| | | | without CT, | | expression, | benefit from |
| | | | and | | patients did not | RT |
| | | | HR +ve, not | | benefit from | |
| | | | TNBC, not | | RT after BCS | |
| | | | HER2 +ve | | | |
| | | | with or without | | | |
| | | | Radiotherapy | | | |
| Class | Biomarker: | Quantified | Patients treated | In patient | Not | Not |
| 2/ | Tumoral IRF9: | amount of | with BCS | population, | statistically | statistically |
| Table | Elevated | expression of | without CT, | elevated | significant in | significant in |
| 5 | biomarker | IRF9 in the | and | expression vs low | joint analysis | joint analysis |
| | expression is | tumor | HR +ve, not | expression of | in patient | in patient |
| | defined as the | compartment | TNBC, not | biomarker not | population | population |
| | quantity of | cells (H- | HER2 +ve, not | prognostic for | | |
| | biomarker | SCORE) | node positive | recurrence risk | | |
| | expression in the | | and | after BCS and not | | |
| | tumor | | biomarker had | predictive for RT | | |
| | compartment | | known | benefit | | |
| | (eg. H-SCORE) | | expression | | | |
| | that is greater | | with or without | | | |
| | than 25 | | Radiotherapy | | | |
| | percentile of the | | | | | |
| | patient | | | | | |
| | population. | | | | | |
| | Low expression | | | | | |
| | is not elevated | | | | | |
| | expression. | | | | | |
| Class | Biomarker | Method of | Patients treated | In patient | Elevated vs | NA |
| 3/ | Algorithm: | class 1. and | with BCS | population, | low tumoral | |
| Table | Simple linear | method of class | without CT, | patients benefited | IRF9 | |
| 6 | model | 2., respectively | and | from RT with | expression was | |
| | combining class | | HR +ve, not | significantly | significantly | |
| | 1 biomarker. | | TNBC, not | lower rates of | associated with | |
| | with class 2. | | HER2 +ve, not | recurrence, | increased risk | |
| | biomarker: | | node positive | elevated | of recurrence | |
| | tumoral | | and | expression vs low | But, | |
| | IRF9 >25 | | both | expression of | Elevated vs | |
| | percentile | | biomarkers had | stromal PD1 | low stromal | |
| | and/or stromal | | known | biomarker not | PD1/stromal | |
| | PD1 >25 | | expression | significantly | surface area | |
| | percentile, using | | with or without | associated with | not | |
| | definitions from | | Radiotherapy | recurrence risk | significantly | |
| | class 1. and | | | after BCS but | associated with | |
| | class 2., | | | elevated IRF9 | increased risk | |
| | respectively | | | associated with | of recurrence | |
| | | | | WORSE | | |
| | | | | outcome. | | |
| Class | Biomarker | Method of 1. | Patients treated | In patient | Not | Not |
| 4/ | Algorithm: | and method of | with BCS | population, | statistically | statistically |
| Table | model | 2., respectively | without CT, | elevated | significant in | significant in |
| 7A | combining class | | and | expression vs low | joint analysis | joint analysis |
| | 1 biomarker. | | HR +ve, not | expression of | in patient | in patient |
| | with class 2. | | TNBC, not | biomarker not | population | population |
| | biomarker: | | HER2 +ve, not | prognostic for | | |
| | stromal | | node positive | recurrence risk | | |
| | PD1 >25 | | and | after BCS and not | | |
| | percentile | | both | predictive for RT | | |
| | AND tumoral | | biomarkers had | benefit | | |
| | IRF9 <25 | | known | | | |
| | percentile, using | | expression, | | | |
| | definitions from | | AND Tumoral | | | |
| | class 1. and | | IRF9 | | | |
| | class 2., | | expression was | | | |
| | respectively | | low ≤25 | | | |
| | | | percentile of | | | |
| | | | population | | | |
| | | | with or without | | | |
| | | | Radiotherapy | | | |
| Class | Biomarker | Method of 1. | Patients treated | In patient | Not | Not |
| 4.1/ | Algorithm: | and method of | with BCS | population, | statistically | statistically |
| Table | model | 2., respectively | without CT, | elevated | significant in | significant in |
| 7B | combining class | | and | expression vs low | joint analysis | joint analysis |
| | 1 biomarker. | | HR +ve, not | expression of | in patient | in patient |
| | with class 2. | | TNBC, not | biomarker not | population | population |
| | biomarker: | | HER2 +ve, not | prognostic for | | |
| | stromal | | node positive | recurrence risk | | |
| | PD1 >25 | | and | after BCS and not | | |
| | percentile | | both | predictive for RT | | |
| | AND tumoral | | biomarkers had | benefit | | |
| | IRF9 <50 | | known | | | |
| | percentile, using | | expression, | | | |
| | definitions from | | AND Tumoral | | | |
| | class 1. | | IRF9 | | | |
| | | | expression was | | | |
| | | | not high (≤50 | | | |
| | | | percentile of | | | |
| | | | population) | | | |
| | | | with or without | | | |
| | | | Radiotherapy | | | |
| Class | Biomarker | Method of 1. | Patients treated | In patient | Not | Not |
| 4.2/ | Algorithm: | and method of | with BCS | population, | statistically | statistically |
| Table | model | 2., respectively | without CT, | elevated | significant in | significant in |
| 8 | combining class | | and | expression vs low | joint analysis | joint analysis |
| | 1 biomarker. | | HR +ve, not | expression of | in patient | in patient |
| | with class 2. | | TNBC, not | stromal PD1 | population | population |
| | biomarker: | | HER2 +ve, not | biomarker not | | |
| | stromal | | node positive | prognostic for | | |
| | PD1 >25 | | and | recurrence risk | | |
| | percentile, | | both | after BCS and not | | |
| | using definition | | biomarkers had | predictive for RT | | |
| | of class1. | | known | benefit | | |
| | | | expression, | | | |
| | | | AND Tumoral | | | |
| | | | IRF9 | | | |
| | | | expression was | | | |
| | | | elevated (>25 | | | |
| | | | percentile of | | | |
| | | | population) | | | |
| | | | using | | | |
| | | | definition of | | | |
| | | | class 2. | | | |
| | | | with or without | | | |
| | | | Radiotherapy | | | |
| Class | Non-Linear | Method of 1. | Same as 3. | Non-linear joint | Statistically | Statistically |
| 5/ | Biomarker | and method of | | (PD1 AND IRF9) | significant in | significant in |
| Table | Algorithm: | 2., respectively | | term was | joint analysis | joint analysis |
| 9 | Non-linear | | | prognostic for | in patient | in patient |
| | integration of | | | recurrence after | population | population |
| | class 1 | | | BCS and | | |
| | biomarker. and | | | predictive for RT | | |
| | class 2. | | | benefit | | |
| | biomarker: | | | | | |
| | Elevated stromal | | | | | |
| | PD1 | | | | | |
| | expression/ | | | | | |
| | stromal tumor area | | | | | |
| | (>25 percentile | | | | | |
| | of population) | | | | | |
| | AND elevated | | | | | |
| | IRF9 expression | | | | | |
| | quantity (>25 | | | | | |
| | percentile | | | | | |
| | population) | | | | | |
| Class | Same as class 5 | Using MIF and | Same as class 5 | Non-linear joint | Not | Not |
| 6/ | | quantitated the | | (PD1 AND IRF9) | statistically | statistically |
| Table | | percent of cells | | term elevated | significant in | significant in |
| 10A | | positive for | | expression was | joint analysis | joint analysis |
| | | PD1 | | not prognostic for | in patient | in patient |
| | | expression in | | recurrence after | population | population |
| | | the stromal | | BCS and not | | |
| | | compartment, | | predictive for RT | | |
| | | and | | benefit | | |
| | | method of 2., | | | | |
| | | respectively | | | | |
| Class | Same as class 5 | Method of 1. | Same as class 5 | Non-linear joint | Statistically | Statistically |
| 7/ | | and | | (PD1 AND IRF9) | significant in | significant in |
| Table | | using MIF and | | term elevated | joint analysis | joint analysis |
| 10B | | the percent of | | expression was | in patient | in patient |
| | | cells in the | | prognostic for | population | population |
| | | rumor | | recurrence after | | |
| | | compartment | | BCS and | | |
| | | that were | | predictive for RT | | |
| | | positive for | | benefit | | |
| | | IRF9 | | | | |
| | | expression. | | | | |
| Class | IRF9 <2 | (a) and (b) | R +ve, not | | | |
| 8 | percentile and | | TNBC, not | | | |
| | PD1 <25 | | HER2 +ve | | | |
| | percentile | | | | | |
| Class | Biomarker: | Using MIF and | Patients treated | biomarker high | High vs not | NA |
| 9/ | tumoral | quantitated the | with BCS | expression was | High tumoral | |
| Table | ALDHA1 | amount of | without CT, | prognostic for | ALDHA1 | |
| 11A | High biomarker | expression of | and | increased | expression was | |
| | expression is | ALDHA1 in | HR +ve, not | recurrence rate in | significantly | |
| | defined as | the tumor | TNBC, not | population | associated with | |
| | quantity of | compartment | HER2 +ve, not | | increased risk | |
| | ALDHA1 | cells (eg. % | node positive | | of recurrence | |
| | expression in | times intensity, | and | | | |
| | tumor cells, | or H-SCORE) | biomarker had | | | |
| | such as H- | | known | | | |
| | SCORE, for >75 | | expression | | | |
| | percentile of | | with or without | | | |
| | patient | | Radiotherapy | | | |
| | population. | | | | | |
| Class | Biomarker | Method of 1., | Patients treated | ALDHA1 | Statistically | Statistically |
| 10/ | Algorithm: | method of 2., | with BCS | biomarker high | significant in | significant in |
| Table | Non-linear | and method of | without CT, | expression was | joint analysis | joint analysis |
| 12A | model | 9., respectively | and | prognostic for | in patient | in patient |
| | combining class | | HR +ve, not | increased | population | population |
| | 9 biomarker | | TNBC, not | recurrence rate | when | when |
| | with class 1 | | HER2 +ve, not | and predictive for | ALDHA1 | ALDHA1 |
| | biomarker. and | | node positive | RT benefit in | expression is | expression is |
| | class 2. | | and | population only | high and | high and |
| | biomarker: | | IRF9 and PD1 | when Non-linear | stromal PD1 | stromal PD1 |
| | tumor | | biomarkers had | joint (PD1 AND | positive cells/ | positive cells/ |
| | ALDHA1 >75 | | known | IRF9) term had | stromal area is | stromal area |
| | percentile, | | expression | elevated | elevated and | is elevated |
| | stromal | | with or without | expression, but | tumoral IRF9 | and tumoral |
| | PD1 >25 | | Radiotherapy | otherwise was not | expression is | IRF9 |
| | percentile | | | | elevated | expression is |
| | AND tumoral | | | | (methods of | elevated |
| | IRF9 >25 | | | | class 1, 2, and | (methods of |
| | percentile. | | | | 9) | class 1, 2, and |
| | | | | | | 9) |
| Class | Biomarker | Method of 1., | Patients treated | ALDHA1 | Not | Not |
| 10.1/ | Algorithm: | method of 2., | with BCS | biomarker high | statistically | statistically |
| Table | Non-linear | and method of | without CT, | expression was | significant in | significant in |
| 12A | model | 9., respectively | and | not prognostic for | joint analysis | joint analysis |
| | combining class | | HR +ve, not | increased | in patient | in patient |
| | 9 biomarker | | TNBC, not | recurrence rate | population | population |
| | with class 1 | | HER2 +ve, not | when Non-linear | when | when |
| | biomarker. and | | node positive | joint (PD1 AND | ALDHA1 | ALDHA1 |
| | class 2. | | and | IRF9) term did | expression is | expression is |
| | biomarker: | | IRF9 and PD1 | not have elevated | high and either | high and |
| | tumor | | biomarkers had | expression | stromal PD1 | either stromal |
| | ALDHA1 >75 | | known | | positive cells/ | PD1 positive |
| | percentile, | | expression | | stromal area or | cells/stromal |
| | stromal | | with or without | | tumoral IRF9 | area of |
| | PD1 >25 | | Radiotherapy | | expression is | tumoral IRF9 |
| | percentile | | | | not elevated | expression is |
| | AND tumoral | | | | (methods of | not elevated |
| | IRF9 >25 | | | | class 1, 2, and | (methods of |
| | percentile. | | | | 9) | class 1, 2, and |
| | | | | | | 9) |
| Class | Biomarker: | Using MIF and | Patients treated | biomarker high | High vs not | NA |
| 11/ | tumoral GLUT1 | quantitated the | with BCS | expression was | High tumoral | |
| Table | High biomarker | amount of | without CT, | prognostic for | GLUT1 | |
| 11B | expression is | expression of | and | increased | expression was | |
| | defined as | GLUT1 in the | HR +ve, not | recurrence rate in | significantly | |
| | quantity of | tumor | TNBC, not | population | associated with | |
| | GLUT1 | compartment | HER2 +ve, not | | increased risk | |
| | expression in | cells (eg. % | node positive | | of recurrence | |
| | tumor cells, | times intensity, | and | | | |
| | such as H- | or H-SCORE) | biomarker had | | | |
| | SCORE, for >75 | | known | | | |
| | percentile of | | expression | | | |
| | patient | | with or without | | | |
| | population. | | Radiotherapy | | | |
| Class | Biomarker | Method of 1., | Patients treated | Both not-high | Statistically | Statistically |
| 12/ | Algorithm: | method of 2., | with BCS | expression of | significant in | significant in |
| Table | Non-linear | and method of | without CT, | GLUT1 | joint analysis | joint analysis |
| 12B | model | 11., | and | biomarker and | in patient | in patient |
| | combining class | respectively | HR +ve, not | non-elevated joint | population | population |
| | 11 biomarker | | TNBC, not | (PD1 AND IRF9) | when both | when both |
| | with class 1 | | HER2 +ve, not | term expression | GLUT1 | GLUT1 |
| | biomarker. and | | node positive | was prognostic | expression is | expression is |
| | class 2. | | and | for decreased | not high and | not high and |
| | biomarker: | | IRF9 and PD1 | recurrence rate | (either stromal | (either |
| | tumor | | biomarkers had | and predictive for | PD1 positive | stromal PD1 |
| | GLUT1 >75 | | known | diminished RT | cells/stromal | positive cells/ |
| | percentile, | | expression | benefit in | area or tumoral | stromal area |
| | stromal | | with or without | population | IRF9 | or tumoral |
| | PD1 >25 | | Radiotherapy | | expression is | IRF9 |
| | percentile | | | | not elevated) | expression is |
| | AND tumoral | | | | (methods of | not elevated) |
| | IRF9 >25 | | | | cases 1, 2, and | (methods of |
| | percentile. | | | | 9) | cases 1, 2, and |
| | | | | | | 9) |
| Class | Biomarker | Method of 1., | Patients treated | Joint non-linear | Statistically | Statistically |
| 13/ | Algorithm: | method of 2., | with BCS | term of not high | significant in | significant in |
| Table | Non-linear | and method of | without CT, | expression of | joint analysis | joint analysis |
| 13 | model | 9., method of | and | ALDHA1 and not | in patient | in patient |
| | combining class | 11, respectively | HR +ve, not | high expression | population | population |
| | 9 biomarker and | | TNBC, not | of GLUT1 and | when both | When both |
| | class 11 | | HER2 +ve, not | not elevated | ALDHA1 and | ALDHA1 and |
| | biomarker, with | | node positive | expression Non- | GLUT1 | GLUT1 |
| | class 1 | | and | linear joint (PD1 | expression is | expression is |
| | biomarker. and | | combined 4 | AND IRF9) term | not high and | not high and |
| | class 2. | | biomarker term | was prognostic | there is not | there is not |
| | biomarker: | | had known | for decreased | elevated | elevated |
| | tumor | | value | recurrence risk | expression for | expression for |
| | GLUT1 >75 | | with or without | and predictive of | Non-linear | Non-linear |
| | percentile, | | Radiotherapy | diminished RT | joint (PD1 | joint (PD1 |
| | ALDHA1 >75 | | | benefit | AND IRF9) | AND IRF9) |
| | percentile, | | | | term (methods | term |
| | stromal | | | | of cases 1, 2, | (methods of |
| | PD1 >25 | | | | and 9, 11) | cases 1, 2, and |
| | percentile | | | | | 9, 11) |
| | AND tumoral | | | | | |
| | IRF9 >25 | | | | | |
| | percentile. | | | | | |

### Example 1

Analysis was performed during tissue segmentation in the in Form Tissue Finder to differentiate the distinct tissue categories in the tissue images. Herein, we created specific categories such as tumor, stroma, and glass by drawing the training regions (three different pathologists created the specific categories) for each category to train the tissue segmenter. Tissue masks selected for training encompass the entire tissue category types to ensure comprehensive coverage. Tissue category selection was further confirmed based on the stains offering independent pattern information, such as DAPI and Pan-Ck. Finally, the diverse training images created to represent diverse staining levels and tissue architectures were used to train the inform tissue segmenter that processed the images from the whole cohort (Output tissue segmentation was reviewed by two individual pathologists).

T1T2N0M0 patients with invasive breast cancer without TNBC (triple negative breast cancer)- or HER2-enhanced (HER2+) disease who were treated with surgery without RT that had elevated stromal PD1 expression/stromal surface area (>25 percentile of this biomarker population) were not significantly associated with higher risk of local or distant breast cancer recurrence and were not significantly associated with a reduction in recurrence rate from RT (Class 1, Table 4A); these patients may have received a benefit from RT for reducing recurrence rate, but it was not statistically significant in this patient population.

In the subset of the T1T2N0M0 patients with elevated ratio of stromal PD1 expression/stromal surface area (>25 percentile of this biomarker population), there was a significant reduction in recurrence rate from RT (Class 1.1, Table 4B), which means that these patients benefited from RT for reducing the rate of local or distant breast cancer recurrence. However, T1T2N0M0 patients without an elevated ratio of stromal PD1 expression/stromal surface area (<25 percentile of this biomarker population) exhibited a non-significant reduction in the recurrence rate from RT (Class 1.2, Table 4C), which indicates that these patients did not benefit from RT for reducing the rate of local or distant breast cancer recurrence. The population distribution of number of PD1 positive cells per square mm of stromal area is summarized in Table 4D by site, where the expression is normalized between sites by piecewise quartile interpolation of a working site versus a selected reference.

Another biomarker of interest in breast cancer is IRF9, where elevated IRF9 protein expression in triple-negative breast cancer was shown to predict decreased risk of distant metastasis and cancer-related death. The expression of the TRF9 protein in the tumor compartment was determined as either the percent of positive cells expression IRF9 or the amount of the protein present in the tumor as assessed by H-SCORE.

Patients with elevated tumoral IRF9 (>25 percentile of this biomarker population) did not have a statistically increased risk of recurrence and there was not an associated significant benefit from RT (non-significant interaction). Thus, it is counterintuitive to combine elevated IRF9 biomarker and elevated PD1 biomarker expression to identify patients who benefit from RT, based on these non-significant findings of the IRF9 biomarker with respect to RT. (Table 5). Based on the current knowledge of the field, one would expect that elevated IRF9 would be associated with improved outcome, but that is not the case with the results presented here.

In this population, the opposite association between IRF9 and recurrence rate was identified, where higher IRF9 was associated with a higher recurrence rate, not a lower recurrence rate. Furthermore, when including the effect of elevated expression of PD1 (>25% percentile of this biomarker population) and elevated tumoral IRF9 (>25% percentile of this biomarker population) in an additive manner, elevated IRF9 expression but not PD1 expression was associated with an increased rate of recurrence. (Table 6)

TABLE 4A
Biomarker: “stromal PD1/stromal surface area” ratio of the number of cells with PD1
expression in stromal compartment/area of stromal compartment; elevated expression
of biomarker is greater than >25 percentile of the biomarker in the population.
Patients: n = 453, number of events = 76
(4 observations deleted due to missingness)
Patients: from all study sites, node negative, treated with BCS, not treated with
chemotherapy, not HER2+, not triple negative, complete data for biomarker
Outcome: either ipsilateral invasive breast event or distant invasive event
Analysis: Cox Proportional Hazard Analysis
Biomarker: number of cells in stromal compartment with PD1 expression divided
by area of stromal compartment
Elevated Expression: biomarker expression > 25 percentile of biomarker
expression for population

| | coef | exp(coef) | se(coef) | Z | Pr(>\|z\|) |
|---|---|---|---|---|---|
| Biomarker(Stromal PD1/Stromal | 0.9707 | 2.6399 | 0.7505 | 1.293 | 0.1959 |
| Area) > (25% of population) TRUE | | | | | |
| RT TRUE | 0.6159 | 1.8513 | 0.7502 | 0.821 | 0.4116 |
| Interaction RT TRUE: | −1.4078 | 0.2447 | 0.8057 | −1.747 | 0.0806 |
| (Biomarker(Stromal PD1/Stromal | | | | | |
| Area) > (25% of population)) TRUE | | | | | |

Signif. codes: 0 ‘***’ 0.001 ‘**’ 0.01 ‘*’ 0.05 ‘.’ 0.1 ‘ ’ 1

| | exp(coef) | exp(−coef) | lower .95 | upper .95 |
|---|---|---|---|---|
| Biomarker(Stromal PD1/Stromal | 2.6399 | 0.3788 | 0.60636 | 11.493 |
| Area) > (25% of population) TRUE | | | | |
| RT TRUE | 1.8513 | 0.5402 | 0.42554 | 8.054 |
| Interaction RT: | 0.2447 | 4.0870 | 0.05044 | 1.187 |
| (Biomarker(Stromal PD1/Stromal | | | | |
| Area) > (25% of population)) TRUE | | | | |

Concordance = 0.606 (se = 0.033)
Likelihood ratio test = 10.42 on 4 df, p = 0.03
Wald test = 10.82 on 4 df, p = 0.03
Score (logrank) test = 11.24 on 4 df, p = 0.02

TABLE 4B
Subset analysis of patients with elevated expression of biomarker
“stromal PD1/stromal surface area”
Patients: n = 360, number of events = 58
(3 observations deleted due to missingness)
Patients: from all study sites, node negative, treated with BCS, not
treated with chemotherapy, not HER2+, not triple negative,
complete data for biomarker
Patient subset: biomarker >25 percentile of biomarker result for population
Outcome: either ipsilateral invasive breast event or distant invasive event
Analysis: Cox Proportional Hazard Analysis
Biomarker: number of cells in stromal compartment with PD1
expression divided by area of stromal compartment
Elevated Expression: biomarker expression > 25 percentile of biomarker
expression for population

| | coef | exp(coef) | se(coef) | Z | Pr(>\|z\|) |
|---|---|---|---|---|---|
| RT TRUE | −0.7867 | 0.4553 | 0.2952 | −2.665 | 0.0077 ** |

Signif. codes: 0 ‘***’ 0.001 ‘**’ 0.01 ‘*’ 0.05 ‘.’ 0.1 ‘ ’ 1

| | exp(coef) | exp(−coef) | lower .95 | upper .95 |
|---|---|---|---|---|
| RT TRUE | 0.4553 | 2.196 | 0.2553 | 0.8121 |

Concordance = 0.611 (se = 0.036)
Likelihood ratio test = 9.08 on 2 df, p = 0.01
Wald test = 9.71 on 2 df, p = 0.008
Score (logrank) test = 10.16 on 2 df, p = 0.006

TABLE 4C
Subset analysis of patients with not-elevated expression of biomarker
“stromal PD1/stromal surface area”
Patients: n = 93, number of events = 18
(1 observation deleted due to missingness)
Patients: from all study sites, node negative, treated with BCS, not treated
with chemotherapy, not HER2+, not triple negative, complete data for
biomarker
Patient subset: biomarker ≤ 25 percentile of biomarker
result for population
Outcome: either ipsilateral invasive breast event or distant invasive event
Analysis: Cox Proportional Hazard Analysis
Biomarker: number of cells in stromal compartment with PD1 expression
divided by area of stromal compartment
Elevated Expression: biomarker expression > 25 percentile of biomarker
expression for population

| | coef | exp(coef) | se(coef) | Z | Pr(>\|z\|) |
|---|---|---|---|---|---|
| RT TRUE | 0.5977 | 1.8179 | 0.7507 | 0.796 | 0.426 |

| | exp(coef) | exp(−coef) | lower .95 | upper .95 |
|---|---|---|---|---|
| RT TRUE | 1.8179 | 0.5501 | 0.4174 | 7.918 |

Concordance = 0.54 (se = 0.064)
Likelihood ratio test = 0.81 on 2 df, p = 0.7
Wald test = 0.7 on 2 df, p = 0.7
Score (logrank) test = 0.72 on 2 df, p = 0.7

TABLE 4D
PD1 positive expression per mm², population distribution by site
Example of number of cells in stromal compartment with PD1 expression
divided by area of stromal compartment by site and normalized for all sites

| | | | |
|---|---|---|---|
| 25% | 50% | Mean | 75% |

Non-normalized - Distribution by site

| | | | | |
|---|---|---|---|---|
| Site 1 | 329.06 | 645 | 1133 | 1416 |
| Site 2 | 897.50 | 1772 | 1901 | 2635 |
| Site 3 | 731.1 | 1249 | 1611 | 1856 |
| Site 4 | 711.6 | 1014 | 1307 | 1562 |

PD1 positive expression per square mm, population
distribution by site (Normalized by interpolation
by quartile between sites)

| | | | | |
|---|---|---|---|---|
| Site 1 | 828 | 1479 | 1862 | 2553 |
| Site 2-4 | 814 | 1639 | 1814 | 2408 |

TABLE 5
Assessment of biomarker tumoral IRF9 H-SCORE; elevated expression of biomarker
is greater than >25 percentile of the biomarker in the population.
Patients: n = 597, number of events = 91
(4 observations deleted due to missingness)
Patients: from all study sites, node negative, treated with BCS, not
treated with chemotherapy, not HER2+, not triple negative,
complete data for biomarker
Outcome: either ipsilateral invasive breast event or distant invasive event
Analysis: Cox Proportional Hazard Analysis
Biomarker: H-SCORE of IRF9 expression for cells in tumor compartment
Elevated Expression: biomarker expression > 25 percentile of biomarker
expression for population

| | coef | exp(coef) | se(coef) | Z | Pr(>\|z\|) |
|---|---|---|---|---|---|
| Biomarker(tumor IRF9 H-SCORE) > | 1.15256 | 3.16630 | 0.63139 | 1.825 | 0.0679 . |
| (25% of population) TRUE | | | | | |
| RT TRUE | −0.06391 | 0.93809 | 0.64069 | −0.100 | 0.9205 |
| Interaction RT TRUE: | −0.53147 | 0.58774 | 0.70026 | −0.759 | 0.4479 |
| (Biomarker(tumor IRF9 H-SCORE) > | | | | | |
| (25% of population)) TRUE | | | | | |

Signif. codes: 0 ‘***’ 0.001 ‘**’ 0.01 ‘*’ 0.05 ‘.’ 0.1 ‘ ’ 1

| | exp(coef) | exp(−coef) | lower .95 | upper .95 |
|---|---|---|---|---|
| Biomarker(tumor IRF9 H-SCORE) > | 3.1663 | 0.3158 | 0.9186 | 10.9143 |
| (25% of population) TRUE | | | | |
| RT TRUE | 0.9381 | 1.0660 | 0.2672 | 3.2931 |
| Interaction RT TRUE: | 0.5877 | 1.7014 | 0.1490 | 2.3187 |
| (Biomarker(tumor IRF9 H-SCORE) > | | | | |
| (25% of population)) TRUE | | | | |

Concordance = 0.622 (se = 0.029)
Likelihood ratio test = 14.03 on 4 df, p = 0.007
Wald test = 13.95 on 4 df, p = 0.007
Score (logrank) test = 14.53 on 4 df, p = 0.006

TABLE 6
Assessment of linear algorithm of elevated expression of biomarker “stromal PD1/stromal
surface area” (>25 percentile of the population biomarker distribution) and elevated tumoral
IRF9 H-SCORE expression (>25 percentile of population biomarker distribution)
Patients: n = 489, number of events = 78
(6 observations deleted due to missingness)
Patients: from all study sites, node negative, treated with BCS, not treated with chemotherapy,
not HER2+, not triple negative, data complete for both biomarkers
Outcome: either ipsilateral invasive breast event or distant invasive event
Analysis: Cox Proportional Hazard Analysis
Biomarker 1: number of cells in stromal compartment with PD1 expression divided
by area of stromal compartment
Biomarker 2: H-SCORE of IRF9 expression for cells in tumor compartment
Elevated Expression (biomarker 1): biomarker 1 expression > 25 percentile of
biomarker 1 expression for population
Elevated Expression (biomarker 2): biomarker 2 expression > 25 percentile of
biomarker 2 expression for population

| | coef | exp(coef) | se(coef) | Z | Pr(>\|z\|) |
|---|---|---|---|---|---|
| Biomarker 1(Stromal PD1/Stromal | 0.06924 | 1.07170 | 0.27044 | 0.256 | 0.7979 |
| Area) > (25% of population) TRUE | | | | | |
| Biomarker 2 (tumor IRF9 H-SCORE) > | 0.60974 | 1.83995 | 0.30939 | 1.971 | 0.0488 * |
| (25% Of population) TRUE | | | | | |
| RT TRUE | −0.55479 | 0.57420 | 0.24144 | −2.298 | 0.0216 * |

Signif. codes: 0 ‘***’ 0.001 ‘**’ 0.01 ‘*’ 0.05 ‘.’ 0.1 ‘ ’ 1

| | exp(coef) | exp(−coef) | lower .95 | upper .95 |
|---|---|---|---|---|
| Biomarker 1(Stromal PD1/Stromal | 1.0717 | 0.9331 | 0.6308 | 1.8209 |
| Area) > (25% of population) TRUE | | | | |
| Biomarker 2 (tumor IRF9 H-SCORE) > | 1.8400 | 0.5435 | 1.0033 | 3.3741 |
| (25% of population) TRUE | | | | |
| RT TRUE | 0.5742 | 1.7416 | 0.3577 | 0.9217 |

Concordance = 0.615 (se = 0.034)
Likelihood ratio test = 13.06 on 4 df, p = 0.01
Wald test = 12.74 on 4 df, p = 0.01
Score (logrank) test = 13 on 4 df, p = 0.01

### Example 2

In patients with not elevated tumoral IRF9 expression (for example, <25 percentile of population distribution (Table 7A), or for example, <50 percentile of population biomarker 2 distribution (Table 7B), those with an elevated ratio (cells with stromal PD1 expression/stromal area ratio) (>25 percentile of population distribution) did not have significantly increased risk of recurrence. Likewise, in the subset of patients with elevated tumoral IRF9 expression (tumoral IRF9 H-SCORE >25 percentile of population tumoral IRF9 H-SCORE distribution) (Table 8), those with an elevated ratio of stromal PD1/stromal surface area ratio (>25% of population biomarker 1 distribution) did not have a significant increase in the risk of recurrence of a benefit from RT.

TABLE 7A
Assessment of biomarker 1 “stromal PD1/stromal surface area” (>25% of population
biomarker 1 distribution), in subset of population without biomarker 2 tumoral IRF9
H-SCORE elevated expression (<25 percentile of population biomarker 2 distribution)
Patients: n = 86, number of events = 13
(2 observations deleted due to missingness)
Patients: from all study sites, node negative, treated with BCS, not treated with
chemotherapy, not HER2+, not triple negative, data complete for both biomarkers
Patient subset: Not Elevated Expression of Biomarker 2
Outcome: either ipsilateral invasive breast event or distant invasive event
Analysis: Cox Proportional Hazard Analysis
Biomarker 1: number of cells in stromal compartment with PD1 expression divided by area of stromal compartment
Biomarker 2: H-SCORE of IRF9 expression for cells in tumor compartment
Elevated Expression (biomarker 1): biomarker 1 expression > 25 percentile of biomarker 1 expression for population
Elevated Expression (biomarker 2): biomarker 2 expression > 25 percentile of biomarker 2 expression for population

| | coef | exp(coef) | se(coef) | Z | Pr(>\|z\|) |
|---|---|---|---|---|---|
| Biomarker 1(Stromal PD1/Stromal | 0.4500 | 1.5683 | 1.1269 | 0.399 | 0.690 |
| Area) > (25% of population) | | | | | |
| TRUE | | | | | |
| RT TRUE | −0.2638 | 0.7681 | 1.2272 | −0.215 | 0.830 |
| Interaction RT TRUE: Biomarker | −0.2531 | 0.7764 | 1.3872 | −0.182 | 0.855 |
| 1(Stromal PD1/Stromal Area) > | | | | | |
| (25% of population) TRUE | | | | | |

| | exp(coef) | exp(−coef) | lower .95 | upper .95 |
|---|---|---|---|---|
| Biomarker 1(Stromal PD1/Stromal | 1.5683 | 0.6376 | 0.17227 | 14.277 |
| Area) > (25% of population) | | | | |
| TRUE | | | | |
| rtTRUE | 0.7681 | 1.3019 | 0.06932 | 8.511 |
| Interaction RT TRUE: Biomarker | 0.7764 | 1.2881 | 0.05120 | 11.772 |
| 1(Stromal PD1/Stromal Area) > | | | | |
| (25% of population) TRUE | | | | |

Concordance = 0.585 (se = 0.079)
Likelihood ratio test = 0.94 on 4 df, p = 0.9
Wald test = 1 on 4 df, p = 0.9
Score (logrank) test = 1.02 on 4 df, p = 0.9

TABLE 7B
Assessment of biomarker 1 “stromal PD1/stromal surface area” (>25% of
population biomarker 1 distribution), in subset of population without biomarker
2 tumoral IRF9 H-SCORE high expression (<50 percentile of population biomarker 2 distribution)
Patients: n = 242, number of events = 29
(4 observations deleted due to missingness
Patients: from all study sites, node negative, treated with BCS, not treated with
chemotherapy, not HER2+, not triple negative, data complete for both biomarkers
Patient subset: Not High Expression of Biomarker 2
Outcome: either ipsilateral invasive breast event or distant invasive event
Analysis: Cox Proportional Hazard Analysis
Biomarker 1: number of cells in stromal compartment with PD1 expression divided by area of stromal compartment
Biomarker 2: H-SCORE of IRF9 expression for cells in tumor compartment
Elevated Expression (biomarker 1): biomarker 1 expression > 25 percentile of biomarker 1 expression for population
High Expression (biomarker 2): biomarker 2 expression > 50 percentile of biomarker 2 expression for population

| | coef | exp(coef) | se(coef) | Z | Pr(>\|z\|) |
|---|---|---|---|---|---|
| Biomarker 1(Stromal PD1/Stromal | 0.2409 | 1.2724 | 0.8039 | 0.300 | 0.764 |
| Area) > (25% of population) | | | | | |
| TRUE | | | | | |
| RT TRUE | −0.3882 | 0.6783 | 0.8664 | −0.448 | 0.654 |
| Interaction RT TRUE: Biomarker | 0.1489 | 1.1605 | 0.9789 | 0.152 | 0.879 |
| 1(Stromal PD1/Stromal Area) > | | | | | |
| (25% of population) TRUE | | | | | |

| | exp(coef) | exp(−coef) | lower .95 | upper .95 |
|---|---|---|---|---|
| Biomarker 1(Stromal PD1/Stromal | 1.2724 | 0.7859 | 0.2632 | 6.151 |
| Area) > (25% of population) | | | | |
| TRUE | | | | |
| RT TRUE | 0.6783 | 1.4743 | 0.1241 | 3.706 |
| Interaction RT TRUE: Biomarker | 1.1605 | 0.8617 | 0.1704 | 7.905 |
| 1(Stromal PD1/Stromal Area) > | | | | |
| (25% of population) TRUE | | | | |

Concordance = 0.588 (se = 0.057)
Likelihood ratio test = 1.77 on 4 df, p = 0.8
Wald test = 1.7 on 4 df, p = 0.8
Score (logrank) test = 1.72 on 4 df, p = 0.8

TABLE 8
Assessment of biomarker 1 “stromal PD1/stromal surface area” (>25% of
population biomarker 1 distribution), in subset of population with biomarker 2 tumoral
IRF9 H-SCORE elevated expression (>25 percentile of population biomarker 2 distribution)
Patients: n = 403, number of events = 65
(4 observations deleted due to missingness)
Patients: from all study sites, node negative, treated with BCS, not treated with
chemotherapy, not HER2+, not triple negative, data complete for both biomarkers
Patient subset: Elevated Expression of Biomarker 2
Outcome: either ipsilateral invasive breast event or distant invasive event
Analysis: Cox Proportional Hazard Analysis
Biomarker 1: number of cells in stromal compartment with PD1 expression divided by area of stromal compartment
Biomarker 2: H-SCORE of IRF9 expression for cells in tumor compartment
Elevated Expression (biomarker 1): biomarker 1 expression > 25 percentile of biomarker 1 expression for population
Elevated Expression (biomarker 2): biomarker 2 expression > 25 percentile of biomarker 2 expression for population

| | coef | exp(coef) | se(coef) | Z | Pr(>\|z\|) |
|---|---|---|---|---|---|
| Biomarker 1(Stromal PD1/Stromal | 0.6737 | 1.9615 | 0.5530 | 1.218 | 0.2231 |
| Area) > (25% of population) | | | | | |
| TRUE | | | | | |
| RT TRUE | 0.1636 | 1.1778 | 0.5853 | 0.280 | 0.7798 |
| Interaction RT TRUE: Biomarker | −0.9883 | 0.3722 | 0.6542 | −1.511 | 0.1309 |
| 1(Stromal PD1/Stromal Area) > | | | | | |
| (25% of population) TRUE | | | | | |

| Signif. codes: 0 ‘***’ 0.001 ‘**’ 0.01 ‘*’ 0.05 ‘.’ 0.1 ‘ ’ 1 | | | | |
|---|---|---|---|---|
| | exp(coef) | exp(−coef) | lower .95 | upper .95 |
| Biomarker 1(Stromal PD1/Stromal | 1.9615 | 0.5098 | 0.6636 | 5.7980 |
| Area) > (25% of population) | | | | |
| TRUE | | | | |
| RT TRUE | 1.1778 | 0.8490 | 0.3740 | 3.7093 |
| Interaction RT TRUE: Biomarker | 0.3722 | 2.6866 | 0.1033 | 1.3417 |
| 1(Stromal PD1/Stromal Area) > | | | | |
| (25% of population) TRUE | | | | |

Concordance = 0.627 (se = 0.036)
Likelihood ratio test = 13.49 on 4 df, p = 0.009
Wald test = 13.42 on 4 df, p = 0.009
Score (logrank) test = 13.96 on 4 df, p = 0.007

### Example 3

In an interaction analysis requiring a non-linear interaction of the two biomarkers (stromal PD, and tumoral IRF9 expression), in which the ratio of the number of cells expressing stromal PD1/stromal surface area is elevated (>25 percentile of this biomarker A population) and tumoral IRF9 expression (H-SCORE) is elevated (>25% percentile of this biomarker 2 population), patients with both biomarkers jointly elevated had increased risk of recurrence and had a significant benefit from RT (significant interaction) and otherwise did not have a statistically significant benefit from RT (Table 9).

TABLE 9
Assessment of non-linear biomarker algorithm using biomarker 1 “stromal
PD1/stromal surface area” and biomarker 2 tumoral IRF9 H-SCORE
Patients: n = 838, number of events = 128
(6 observations deleted due to missingness)
Patients: from all study sites, node negative, treated with BCS, not treated with
chemotherapy, not HER2+, not triple negative
Outcome: either ipsilateral invasive breast event or distant invasive event
Analysis: Cox Proportional Hazard Analysis
Biomarker 1: number of cells in stromal compartment with PD1 expression divided by area of stromal compartment
Biomarker 2: H-SCORE of IRF9 expression for cells in tumor compartment
Elevated Expression (biomarker 1): biomarker 1 expression > 25 percentile of biomarker 1 expression for population
Elevated Expression (biomarker 2): biomarker 2 expression > 25 percentile of biomarker 2 expression for population
Biomarker Algorithm (pd1.irf9): elevated expression of biomarker 1 AND elevated expression of biomarker 2

| | coef | exp(coef) | se(coef) | Z | Pr(>\|z\|) |
|---|---|---|---|---|---|
| pd1.irf9 TRUE | 1.0561 | 2.8751 | 0.3650 | 2.893 | 0.00381 ** |
| RT TRUE | 0.1058 | 1.1116 | 0.3050 | 0.347 | 0.72859 |
| Interaction pd1.irf9 | −0.9156 | 0.4003 | 0.4233 | −2.163 | 0.03054 * |
| TRUE: RT TRUE | | | | | |

Signif. codes: 0 ‘***’ 0.001 ‘**’ 0.01 ‘*’ 0.05 ‘.’ 0.1 ‘ ’ 1

| | exp(coef) | exp(−coef) | lower .95 | upper .95 |
|---|---|---|---|---|
| pd1.irf9 TRUE | 2.8751 | 0.3478 | 1.4058 | 5.8798 |
| RT TRUE | 1.1116 | 0.8996 | 0.6115 | 2.0208 |
| Interaction pd1.irf9 | 0.4003 | 2.4984 | 0.1746 | 0.9176 |
| TRUE: rt TRUE | | | | |

Concordance = 0.578 (se = 0.027)
Likelihood ratio test = 15.6 on 4 df, p = 0.004
Wald test = 17.21 on 4 df, p = 0.002
Score (logrank) test = 17.88 on 4 df, p = 0.001

Importantly, when stromal PD was evaluated as the % of stromal cells with positive PD1 expression, instead of using a density of positive PD1 cells/area, those with a >25 percentile of the population percent of cells positive distribution indicate that the joint PD1 and IRF9 biomarker interaction was not significant for recurrence risk or RT benefit. (Class 6, Table 10A). When tumoral IRF9 was evaluated as the of IRF9 cells showing a positive expression, instead of an intensity score of tumor IRF9 protein expression (H-SCORE), those with >25 percentile of the population percent of cells showing a positive distribution of the IRF9 biomarker had an interaction with elevated PD stromal expression that remained significant and had comparable results as with the intensity based evaluation of tumor IRF9. (Table 10B).

TABLE 10A
Assessment of non-linear biomarker algorithm using biomarker 1 “% of stromal cells with positive PD1
expression” and biomarker 2 tumoral IRF9 H-SCORE, instead of biomarker 1 “stromal PD1/stromal surface area”
Patients: n = 838, number of events = 128
(6 observations deleted due to missingness)
Patients: from all study sites, node negative, treated with BCS, not treated with
chemotherapy, not HER2+, not triple negative
Outcome: either ipsilateral invasive breast event or distant invasive event
Analysis: Cox Proportional Hazard Analysis
Biomarker 1: percent of cells with positive PD1 expression in stromal compartment
Biomarker 2: H-SCORE of IRF9 expression for cells in tumor compartment
Elevated Expression (biomarker 1): biomarker 1 expression > 25 percentile of biomarker 1 expression for population
Elevated Expression (biomarker 2): biomarker 2 expression > 25 percentile of biomarker 2 expression for population
Biomarker Algorithm (pd1.pct.irf9): elevated expression of biomarker 1 AND elevated expression of biomarker 2

| | coef | exp(coef) | se(coef) | Z | Pr(>\|z\|) |
|---|---|---|---|---|---|
| pd1.pct.irf9 TRUE | 0.4311 | 1.5389 | 0.3737 | 1.154 | 0.2487 |
| RT TRUE | −0.1498 | 0.8609 | 0.3002 | −0.499 | 0.6178 |
| (Stromal PD1 expression missing) | −0.3956 | 0.6733 | 0.2301 | −1.719 | 0.0856 . |
| TRUE | | | | | |
| (Tumoral IRF9 expression missing) | 0.2496 | 1.2835 | 0.2489 | 1.003 | 0.3160 |
| TRUE | | | | | |
| Interaction pd1.pct.irf9 TRUE: RT | −0.2514 | 0.7777 | 0.4226 | −0.595 | 0.5519 |
| TRUE | | | | | |

| Signif. codes: 0 ‘***’ 0.001 ‘**’ 0.01 ‘*’ 0.05 ‘.’ 0.1 ‘ ’ 1 | | | | |
|---|---|---|---|---|
| | exp(coef) | exp(−coef) | lower .95 | upper .95 |
| pd1.pct.irf9 TRUE | 1.5389 | 0.6498 | 0.7398 | 3.2012 |
| RT TRUE | 0.8609 | 1.1616 | 0.4780 | 1.5506 |
| (Stromal PD1 expression missing) | 0.6733 | 1.4852 | 0.4289 | 1.0569 |
| TRUE | | | | |
| (Tumoral IRF9 expression missing) | 1.2835 | 0.7791 | 0.7880 | 2.0908 |
| TRUE | | | | |
| Interaction pd1.pct.irf9 TRUE: RT | 0.7777 | 1.2858 | 0.3397 | 1.7804 |
| TRUE | | | | |

Concordance = 0.609 (se = 0.027)
Likelihood ratio test = 14.99 on 6 df, p = 0.02
Wald test = 14.49 on 6 df, p = 0.02
Score (logrank) test = 14.76 on 6 df, p = 0.02

TABLE 10B
Assessment of non-linear biomarker algorithm using biomarker 1 “% of stromal cells with positive PD1
expression” and biomarker 2 tumoral IRF9 percent positive, instead of biomarker 2 tumoral IRF9 H-SCORE
Patients: n = 838, number of events = 128
(6 observations deleted due to missingness)
Patients: from all study sites, node negative, treated with BCS, not treated with
chemotherapy, not HER2+, not triple negative
Outcome: either ipsilateral invasive breast event or distant invasive event
Analysis: Cox Proportional Hazard Analysis
Biomarker 1: number of cells in stromal compartment with PD1 expression divided by area of stromal compartment
Biomarker 2: Percent of cells with positive IRF9 expression in tumor compartment
Elevated Expression (biomarker 1): biomarker 1 expression > 25 percentile of biomarker 1 expression for population
Elevated Expression (biomarker 2): biomarker 2 expression > 25 percentile of biomarker 2 expression for population
Biomarker Algorithm (pd1.irf9.pct): elevated expression of biomarker 1 AND elevated expression of biomarker 2

| | coef | exp(coef) | se(coef) | Z | Pr(>\|z\|) |
|---|---|---|---|---|---|
| pd1.irf9.pct TRUE | 0.9540 | 2.5960 | 0.3746 | 2.546 | 0.0109 * |
| RT TRUE | 0.1200 | 1.1275 | 0.3109 | 0.386 | 0.6996 |
| (Stromal PD1 expression missing) | −0.3449 | 0.7083 | 0.2287 | −1.508 | 0.1315 |
| TRUE | | | | | |
| (Tumoral IRF9 expression missing) | 0.2093 | 1.2329 | 0.2487 | 0.842 | 0.4000 |
| TRUE | | | | | |
| Interaction pd1.irf9.pct | −0.9128 | 0.4014 | 0.4279 | −2.133 | 0.0329 * |
| TRUE: RT TRUE | | | | | |

| Signif. codes: 0 ‘***’ 0.001 ‘**’ 0.01 ‘*’ 0.05 ‘.’ 0.1 ‘ ’ 1 | | | | |
|---|---|---|---|---|
| | exp(coef) | exp(−coef) | lower .95 | upper .95 |
| pd1.irf9.pctTRUE | 2.5960 | 0.3852 | 1.2457 | 5.4100 |
| RT TRUE | 1.1275 | 0.8869 | 0.6130 | 2.0738 |
| (Stromal PD1 expression missing) | 0.7083 | 1.4118 | 0.4525 | 1.1088 |
| TRUE | | | | |
| (Tumoral IRF9 expression missing) | 1.2329 | 0.8111 | 0.7572 | 2.0074 |
| TRUE | | | | |
| Interaction pd1.irf9.pct | 0.4014 | 2.4914 | 0.1735 | 0.9285 |
| TRUE: RT TRUE | | | | |

Concordance = 0.601 (se = 0.027)
Likelihood ratio test = 18.71 on 6 df, p = 0.005
Wald test = 19.79 on 6 df, p = 0.003
Score (logrank) test = 20.6 on 6 df, p = 0.002

### Example 4

Patients with an high tumoral ALDHA1 of >75 percentile of the population, by intensity of ALDHA1 expression (such as H-SCORE), had a higher risk of recurrence (class 9, Table 1A). Patients with an elevated tumoral GLUT1 of >75 percentile of the population, by intensity of GLUT1 expression (H-SCORE), had a higher risk of recurrence. (class 11, Table 11B).

TABLE 11A
Assessment of biomarker tumoral ALDHA1 H-SCORE; elevated expression of biomarker
is greater than >75 percentile of the biomarker in the population.
Patients: n = 719, number of events = 105
(9 observations deleted due to missingness)
Patients: from all study sites, node negative, treated with BCS, not treated with
chemotherapy, not HER2+, not triple negative, data complete for biomarker
Outcome: either ipsilateral invasive breast event or distant invasive event
Analysis: Cox Proportional Hazard Analysis
Biomarker: tumoral ALDHA1 H-SCORE
High Expression (biomarker 1): biomarker 1 expression > 75 percentile of biomarker 1 expression for population

| | coef | exp(coef) | se(coef) | Z | Pr(>\|z\|) |
|---|---|---|---|---|---|
| Biomarker (ALDHA1 H-SCORE) > | 0.97837 | 2.66012 | 0.38591 | 2.535 | 0.01124 * |
| (75 percentile of biomarker | | | | | |
| population) TRUE | | | | | |
| RT TRUE | −0.05743 | 0.94419 | 0.27960 | −0.205 | 0.83726 |
| AGE > 55TRUE | −0.15763 | 0.85416 | 0.20659 | −0.763 | 0.44545 |
| Tumor Grade > 5TRUE | 0.57373 | 1.77487 | 0.20748 | 2.765 | 0.00569 ** |
| Tumor Size > T1 TRUE | 0.97835 | 2.66007 | 0.27786 | 3.521 | 0.00043 *** |

Signif. codes: 0 ‘***’ 0.001 ‘**’ 0.01 ‘*’ 0.05 ‘.’ 0.1 ‘ ’ 1

| | exp(coef) | exp(−coef) | lower .95 | upper .95 |
|---|---|---|---|---|
| Biomarker (ALDHA1 H-SCORE) > | 2.6601 | 0.3759 | 1.2486 | 5.6675 |
| (75 percentile of biomarker | | | | |
| population) TRUE | | | | |
| RT TRUE | 0.9442 | 1.0591 | 0.5458 | 1.6333 |
| AGE > 55TRUE | 0.8542 | 1.1707 | 0.5698 | 1.2805 |
| Tumor Grade > 5TRUE | 1.7749 | 0.5634 | 1.1818 | 2.6655 |
| Tumor Size > T1 TRUE | 2.6601 | 0.3759 | 1.5431 | 4.5857 |

Concordance = 0.659 (se = 0.029)
Likelihood ratio test = 31.81 on 7 df, p = 4e−05
Wald test = 35.69 on 7 df, p = 8e−06
Score (logrank) test = 37.91 on 7 df, p = 3e−06

TABLE 11B
Assessment of biomarker tumoral GLUT1 H-
SCORE; elevated expression of biomarker is greater
than >75 percentile of the biomarker in the population.
Patients: n = 736, number of events = 115
(9 observations deleted due to missingness)
Patients: from all study sites, node negative, treated
with BCS, not treated with chemotherapy, not HER2+,
not triple negative, data complete for biomarker
Outcome: either ipsilateral invasive breast event or distant invasive event
Analysis: Cox Proportional Hazard Analysis
Biomarker; tumoral GLUT1 H-SCORE
High Expression (biomarker 1): biomarker expression >75
percentile of biomarker 1 expression for population

| | | exp | se | | Pr |
|---|---|---|---|---|---|
| | coef | (coef) | (coef) | Z | (>\|z\|) |
| Biomarker | 1.1485 | 3.1535 | 0.3892 | 2.951 | 0.003167 ** |
| (GLUT1 H- | | | | | |
| SCORE) > (75 | | | | | |
| percentile of | | | | | |
| biomarker | | | | | |
| population) | | | | | |
| TRUE | | | | | |
| RT TRUE | 0.2034 | 1.2255 | 0.2770 | 0.734 | 0.462792 |
| AGE > | −0.1909 | 0.8262 | 0.1946 | −0.981 | 0.326635 |
| 55TRUE | | | | | |
| Tumor Grade > | 0.7478 | 2.1123 | 0.2019 | 3.704 | 0.000213 *** |
| 5TRUE | | | | | |
| Tumor Size > | 1.0539 | 2.8687 | 0.2611 | 4.036 | 5.43e−05 *** |
| T1 TRUE | | | | | |

Signif. codes: 0 ‘***’ 0.001 ‘**’ 0.01 ‘*’ 0.05 ‘.’ 0.1 ‘ ’ 1

| | exp | exp | lower | upper |
|---|---|---|---|---|
| | (coef) | (−coef) | .95 | .95 |
| Biomarker | 3.1535 | 0.3171 | 1.4707 | 6.7618 |
| (GLUT1 H- | | | | |
| SCORE) > (75 | | | | |
| percentile of | | | | |
| biomarker | | | | |
| population) | | | | |
| TRUE | | | | |
| RT TRUE | 1.2255 | 0.8160 | 0.7121 | 2.1091 |
| AGE > | 0.8262 | 1.2103 | 0.5643 | 1.2099 |
| 55TRUE | | | | |
| Tumor Grade > | 2.1123 | 0.4734 | 1.4220 | 3.1378 |
| 5TRUE | | | | |
| Tumor Size > | 2.8687 | 0.3486 | 1.7197 | 4.7855 |
| T1 TRUE | | | | |

Concordance = 0.678 (se = 0.027)
Likelihood ratio test = 41.54 on 7 df, p = 6e−07
Wald test = 46.41 on 7 df, p = 7e−08
Score (logrank) test = 49.3 on 7 df, p = 2e−08

Importantly, patients with an elevated tumoral ALDHA1 of >75 percentile of the population, by intensity of ALDHA1 expression (H-SCORE), who had jointly elevated ratio of number of cells expression stromal PD1/stromal surface area (>25 percentile of this biomarker population) and elevated level of tumoral IRF9 by H-SCORE (>25 percentile of this biomarker population) had a significantly higher recurrence risk and otherwise did not benefit from RT (class 10. Table 12A).

Neither patients with elevated tumoral ALDHA1 of >75 percentile of the population by intensity of ALDHA1 expression, who had both stromal PD1 elevated (>25 percentile of this biomarker population) and tumoral IRF9 elevated (>25 percentile of this biomarker population) and otherwise had a significant benefit from RT for reduction in recurrence rate (Table 12A).

Importantly, patients with high tumoral GLUT1 of >75 percentile of the population by intensity of GLUT1 expression (H-score), who have a jointly elevated ratio of number of stromal cells expressing PD1/stromal surface area (>25 percentile of this biomarker population) and an elevated tumoral IRF9 expression by H-SCORE (>25 percentile of population) rad a significantly higher recurrence rate and a significant RT benefit (class 12. Table 12B).

TABLE 12A
Assessment of non-linear biomarker algorithm using biomarker 1 “stromal PD1/stromal
surface area” and biomarker 2 tumoral IRF9 H-SCORE and biomarker 3 tumoral ALDHA1 H-SCORE
Patients: n = 572, number of events = 90
(9 observations deleted due to missingness)
Patients: from all study sites, node negative, treated with BCS, not treated with
chemotherapy, not HER2+, not triple negative
Outcome: either ipsilateral invasive breast event or distant invasive event
Analysis: Cox Proportional Hazard Analysis
Biomarker 1: number of cells in stromal compartment with PD1 expression divided by area of stromal compartment
Biomarker 2: H-SCORE of IRF9 expression in tumor compartment
Biomarker 3: H-SCORE of ALDHA1 expression in tumor compartment
Elevated Expression (biomarker 1): biomarker 1 expression > 25 percentile of biomarker 1 expression for population
Elevated Expression (biomarker 2): biomarker 2 expression > 25 percentile of biomarker 2 expression for population
High Expression (biomarker 3): biomarker 3 expression > 75 percentile of biomarker 3 expression for population
Biomarker Algorithm (pdvar): elevated expression of biomarker 1 AND elevated expression of biomarker 2 = elevated,
not elevated expression of biomarker 1 OR not elevated expression of biomarker 2 = not elevated, otherwise missing;
Biomarker Algorithm (aldha1.pdvar.1) = high biomarker 3 expression and pdvar not elevated = FALSE
Biomarker Algorithm (aldha1.pdvar.1) = high biomarker 3 expression and pdvar elevated = TRUE

| | coef | exp(coef) | se(coef) | Z | Pr(>\|z\|) |
|---|---|---|---|---|---|
| aldha1.pdvar.1 TRUE | 1.19283 | 3.29639 | 0.41340 | 2.885 | 0.00391 ** |
| RT TRUE | −0.09264 | 0.91152 | 0.28910 | −0.320 | 0.74863 |
| aldha1.pdvar.1 FALSE | 0.45227 | 1.57188 | 0.74845 | 0.604 | 0.54566 |
| AGE > 55TRUE | −0.02152 | 0.97871 | 0.22833 | −0.094 | 0.92493 |
| Tumor Grade > 5TRUE | 0.60756 | 1.83595 | 0.22327 | 2.721 | 0.00650 ** |
| Size > T1 TRUE | 0.93038 | 2.53547 | 0.30375 | 3.063 | 0.00219 ** |
| aldha1.pdvar.1 TRUE | −0.90839 | 0.40317 | 0.51063 | −1.779 | 0.07525 . |
| aldha1.pdvar.1 FALSE | −0.90998 | 0.40253 | 1.04312 | −0.872 | 0.38301 |

Signif. codes: 0 ‘***’ 0.001 ‘**’ 0.01 ‘*’ 0.05 ‘.’ 0.1 ‘ ’ 1

| | exp(coef) | exp(−coef) | lower .95 | upper .95 |
|---|---|---|---|---|
| aldha1.pdvar.1 TRUE | 3.2964 | 0.3034 | 1.46605 | 7.4119 |
| RT TRUE | 0.9115 | 1.0971 | 0.51723 | 1.6064 |
| aldha1.pdvar.1 FALSE | 1.5719 | 0.6362 | 0.36253 | 6.8156 |
| AGE > 55TRUE | 0.9787 | 1.0217 | 0.62561 | 1.5311 |
| Tumor Grade > 5TRUE | 1.8359 | 0.5447 | 1.18525 | 2.8439 |
| Size > T1 TRUE | 2.5355 | 0.3944 | 1.39799 | 4.5985 |
| aldha1.pdvar.1 TRUE | 0.4032 | 2.4803 | 0.14820 | 1.0968 |
| aldha1.pdvar.1 FALSE | 0.4025 | 2.4843 | 0.05211 | 3.1096 |

Concordance = 0.67 (se = 0.031)
Likelihood ratio test = 30.09 on 9 df, p = 4e−04
Wald test = 34.18 on 9 df, p = 8e−05
Score (logrank) test = 37.07 on 9 df, p = 3e−05

TABLE 12B
Assessment of non-linear biomarker algorithm using biomarker 1 “stromal PD1/stromal
surface area” and biomarker 2 tumoral IRF9 H-SCORE and biomarker 3 tumoral GLUT1 H-SCORE
Patients: n = 838, number of events = 129
(10 observations deleted due to missingness)
Patients: from all study sites, node negative, treated with BCS, not treated with
chemotherapy, not HER2+, not triple negative
Outcome: either ipsilateral invasive breast event or distant invasive event
Analysis: Cox Proportional Hazard Analysis
Biomarker 1: number of cells in stromal compartment with PD1 expression divided by area of stromal compartment
Biomarker 2: H-SCORE of IRF9 expression in tumor compartment
Biomarker 3: H-SCORE of GLUT1 expression in tumor compartment
Elevated Expression (biomarker 1): biomarker 1 expression > 25 percentile of biomarker 1 expression for population
Elevated Expression (biomarker 2): biomarker 2 expression > 25 percentile of biomarker 2 expression for population
High Expression (biomarker 3): biomarker 3 expression > 75 percentile of biomarker 3 expression for population
Biomarker Algorithm (pdvar): elevated expression of biomarker 1 AND elevated expression of biomarker 2 = elevated,
not elevated expression of biomarker 1 OR not elevated expression of biomarker 2 = not elevated, otherwise missing;
Biomarker Algorithm (not.glut1.pdvar.0): not high biomarker 3 expression and pdvar not elevated = TRUE
Biomarker Algorithm (not.glut1.pdvar.0): not high biomarker 3 expression and pdvar elevated = FALSE

| | coef | exp(coef) | se(coef) | Z | Pr(>\|z\|) |
|---|---|---|---|---|---|
| not.glut1.pdvar.0 FALSE | −0.3814 | 0.6829 | 0.4205 | −0.907 | 0.364417 |
| RT TRUE | −1.0587 | 0.3469 | 0.3831 | −2.763 | 0.005722 ** |
| not.glut1.pdvar.0 TRUE | −1.5796 | 0.2061 | 0.4663 | −3.388 | 0.000704 *** |
| AGE > 55TRUE | −0.3348 | 0.7155 | 0.1836 | −1.823 | 0.068234 . |
| Tumor Grade > 5 TRUE | 0.5440 | 1.7230 | 0.1857 | 2.929 | 0.003398 ** |
| Tumor Size > T1 | 0.9457 | 2.5745 | 0.2505 | 3.775 | 0.000160 *** |
| Interaction not.glut1.pdvar.0 | 0.3854 | 1.4703 | 0.5205 | 0.741 | 0.458982 |
| FALSE:: RT TRUE | | | | | |
| Interaction not.glut1.pdvar.0 | 1.5877 | 4.8923 | 0.5366 | 2.959 | 0.003090 ** |
| TRUE: RT TRUE | | | | | |

| Signif. codes: 0 ‘***’ 0.001 ‘**’ 0.01 ‘*’ 0.05 ‘.’ 0.1 ‘ ’ 1 | | | | |
|---|---|---|---|---|
| | exp(coef) | exp(−coef) | lower .95 | upper .95 |
| not.glut1.pdvar.0 FALSE | 0.6829 | 1.4643 | 0.29953 | 1.5570 |
| RT TRUE | 0.3469 | 2.8825 | 0.16373 | 0.7351 |
| not.glut1.pdvar.0 TRUE | 0.2061 | 4.8530 | 0.08263 | 0.5139 |
| AGE > 55TRUE | 0.7155 | 1.3977 | 0.49921 | 1.0254 |
| Tumor Grade > 5 TRUE | 1.7230 | 0.5804 | 1.19725 | 2.4795 |
| Tumor Size > T1 | 2.5745 | 0.3884 | 1.57563 | 4.2066 |
| Interaction not.glut1.pdvar.0 | 1.4703 | 0.6801 | 0.53008 | 4.0780 |
| FALSE: RT TRUE | | | | |

Concordance = 0.665 (se = 0.027)
Likelihood ratio test = 43.06 on 9 df, p = 2e−06
Wald test = 48.31 on 9 df, p = 2e−07
Score (logrank) test = 51.12 on 9 df, p = 7e−08

Patients who had a true non-linear algorithm term defined as: 1) not elevated tumoral ALDHA1>75 percentile of this biomarker population by intensity of ALDHA1 expression AND 2) not elevated tumoral GLUT1>75 percentile of this biomarker population by intensity of GLUT1 expression, AND 3) there was not both positive for ratio of number of stromal cells expressing PD/stromal surface area (>25 percentile of this biomarker population) and tumoral IRF9 elevated (>25 percentile of this biomarker population), had a lower risk of recurrence and had a diminished response to RT (Table 13). This table shows that RT had a significant relative risk reduction in general but not in this specific subset of patients.

TABLE 13
Assessment of non-linear biomarker algorithm “gap.0” using biomarker 1
“stromal PD1/stromal surface area” and biomarker 2 tumoral IRF9 H-SCORE
and biomarker 3 tumoral ALDHA1 H-SCORE and biomarker 4 tumoral GLUT1 H-SCORE
Patients: n = 625, number of events = 95
(8 observations deleted due to missingness)
Patients: from all study sites, node negative, treated with BCS, not treated with
chemotherapy, not HER2+, not triple negative, and biomarker algorithm complete
Outcome: either ipsilateral invasive breast event or distant invasive event
Analysis: Cox Proportional Hazard Analysis
Biomarker 1: number of cells in stromal compartment with PD1 expression divided by area of stromal compartment
Biomarker 2: H-SCORE of IRF9 expression in tumor compartment
Biomarker 3: H-SCORE of ALDHA1 expression in tumor compartment
Biomarker 4: H-SCORE of GLUT1 expression in tumor compartment
Elevated Expression (biomarker 1): biomarker 1 expression > 25 percentile of biomarker 1 expression for population
Elevated Expression (biomarker 2): biomarker 2 expression > 25 percentile of biomarker 2 expression for population
High Expression (biomarker 3): biomarker 3 expression > 75 percentile of biomarker 3 expression for population
High Expression (biomarker 4): biomarker 4 expression > 75 percentile of biomarker 4 expression for population
Biomarker Algorithm (pdvar): elevated expression of biomarker 1 AND elevated expression of biomarker 2 = elevated,
not elevated expression of biomarker 1 OR not elevated expression of biomarker 2 = not elevated, otherwise missing;
Biomarker Algorithm (gap.0): not high biomarker 4 expression and not high biomarker 3 expression and pdvar not elevated = TRUE

| | coef | exp(coef) | se(coef) | Z | Pr(>\|z\|) |
|---|---|---|---|---|---|
| gap.0 TRUE | −1.2770 | 0.2789 | 0.4943 | −2.583 | 0.00978 ** |
| RT TRUE | −0.8177 | 0.4414 | 0.2597 | −3.149 | 0.00164 ** |
| Age > 55TRUE | −0.1626 | 0.8500 | 0.2177 | −0.747 | 0.45510 |
| Tumor Grade > 5 TRUE | 0.5208 | 1.6834 | 0.2153 | 2.419 | 0.01555 * |
| Tumor Size > T1 TRUE | 0.8802 | 2.4114 | 0.2930 | 3.004 | 0.00266 ** |
| Interaction gap.0 TRUE: RT TRUE | 1.3663 | 3.9207 | 0.5642 | 2.422 | 0.01545 * |

Signif. codes: 0 ‘***’ 0.001 ‘**’ 0.01 ‘*’ 0.05 ‘.’ 0.1 ‘ ’ 1

| | exp(coef) | exp(−coef) | lower .95 | upper .95 |
|---|---|---|---|---|
| gap.0 TRUE | 0.2789 | 3.5859 | 0.1058 | 0.7348 |
| RT TRUE | 0.4414 | 2.2653 | 0.2654 | 0.7343 |
| Age > 55TRUE | 0.8500 | 1.1765 | 0.5548 | 1.3022 |
| Tumor Grade > 5 TRUE | 1.6834 | 0.5940 | 1.1040 | 2.5671 |
| Tumor Size > T1 TRUE | 2.4114 | 0.4147 | 1.3580 | 4.2821 |
| Interaction gap.0 TRUE: RT TRUE | 3.9207 | 0.2551 | 1.2975 | 11.8469 |

Concordance = 0.665 (se = 0.031)
Likelihood ratio test = 28.71 on 7 df, p = 2e−04
Wald test = 30.48 on 7 df, p = 8e−05
Score (logrank) test = 31.51 on 7 df, p = 5e−05

Thus, patients who did not have 1) high tumoral ALDHA1>75 percentile of this biomarker population by intensity of ALDHA1 expression AND 2) high tumoral GLUT1>75 percentile of this biomarker population by intensity of GLUT1 expression, AND did not have 3) both stromal PD1 elevated (>25 percentile of this biomarker population) and tumoral IRF9 elevated (>25% percentile of this biomarker population) would not be expected to benefit significantly from RT after BCS (Table 14B). Thus, when these patients have a higher risk profile due to other risk factors, they would be expected to have a diminished response to RT, and are to be treated with RT boost, or an alternative treatment to RT, such as a systemic therapy or mastectomy.

In the counter case to these three are present at high or elevated levels, respectively, patients are expected to benefit from RT leading to improved DFS (Table 14C) with a lower recurrence rate (local or distant) (Table 14D).

TABLE 14A
Lack of benefit of RT for invasive Disease Free Survival (DFS) for patients nonlinear
algorithm “gap.0” true; non-linear biomarker algorithm “gap.0” uses
biomarker 1 “stromal PD1/stromal surface area” and biomarker 2 tumoral IRF9
H-SCORE and biomarker 3 tumoral ALDHA1 H-SCORE and biomarker 4 tumoral GLUT1 H-SCORE
Patients: n = 200, number of events = 35
(3 observations deleted due to missingness)
Patients: from all study sites, mode negative, treated with BCS, not treated with chemotherapy,
not HER2+, not triple negative, and biomarker algorithm complete
Patient subset: biomarker algorithm gap.0 = TRUE
Outcome: either ipsilateral invasive breast event or distant invasive event or contralateral invasive breast event
Analysis: Cox Proportional Hazard Analysis
Biomarker 1: number of cells in stromal compartment with PD1 expression divided by area of stromal compartment
Biomarker 2: H-SCORE of IRF9 expression in tumor compartment
Biomarker 3: H-SCORE of ALDHA1 expression in tumor compartment
Biomarker 4: H-SCORE of GLUT1 expression in tumor compartment
Elevated Expression (biomarker 1): biomarker 1 expression > 25 percentile of biomarker 1 expression for population
Elevated Expression (biomarker 2): biomarker 2 expression > 25 percentile of biomarker 2 expression for population
High Expression (biomarker 3): biomarker 3 expression > 75 percentile of biomarker 3 expression for population
High Expression (biomarker 4): biomarker 4 expression > 75 percentile of biomarker 4 expression for population
Biomarker Algorithm (pdvar): elevated expression of biomarker 1 AND elevated expression of biomarker 2 = elevated,
not elevated expression of biomarker 1 OR not elevated expression of biomarker 2 = not elevated, otherwise missing;
Biomarker Algorithm (gap.0): not high biomarker 4 expression and not high biomarker 3 expression and pdvar not elevated = TRUE

| | coef | exp(coef) | se(coef) | Z | Pr(>\|z\|) |
|---|---|---|---|---|---|
| RT TRUE | 0.32873 | 1.38920 | 0.42439 | 0.775 | 0.439 |

| | exp(coef) | exp(−coef) | lower .95 | upper .95 |
|---|---|---|---|---|
| RT TRUE | 1.389 | 0.7198 | 0.6047 | 3.192 |

Concordance = 0.532 (se = 0.052)
Likelihood ratio test = 0.66 on 2 df, p = 0.7
Wald test = 0.62 on 2 df, p = 0.7
Score (logrank) test = 0.62 on 2 df, p = 0.7

TABLE 14B
Lack of benefit of RT for invasive local and distant metastasis free survival for patients
nonlinear algorithm “gap.0” true; non-linear biomarker algorithm “gap.0”
uses biomarker 1 “stromal PD1/stromal surface area” and biomarker 2 tumoral IRF9
H-SCORE and biomarker 3 tumoral ALDHA1 H-SCORE and biomarker 4 tumoral GLUT1 H-SCORE
Patients: n = 177, number of events = 25
(2 observations deleted due to missingness)
Patients: from all study sites, node negative, treated with BCS, not treated with chemotherapy,
not HER2+, not triple negative, and biomarker algorithm complete
Patient subset: biomarker algorithm gap.0 = TRUE
Outcome: either ipsilateral invasive breast event or distant invasive event
Analysis: Cox Proportional Hazard Analysis
Biomarker 1: number of cells in stromal compartment with PD1 expression divided by area of stromal compartment
Biomarker 2: H-SCORE of IRF9 expression in tumor compartment
Biomarker 3: H-SCORE of ALDHA1 expression in tumor compartment.
Biomarker 4: H-SCORE of GLUT1 expression in tumor compartment.
Elevated Expression (biomarker 1): biomarker 1 expression > 25 percentile of biomarker 1 expression for population
Elevated Expression (biomarker 2): biomarker 2 expression > 25 percentile of biomarker 2 expression for population
High Expression (biomarker 3): biomarker 3 expression > 75 percentile of biomarker 3 expression for population
High Expression (biomarker 4): biomarker 4 expression > 75 percentile of biomarker 4 expression for population
Biomarker Algorithm (pdvar): elevated expression of biomarker 1 AND elevated expression of biomarker 2 = elevated,
not elevated expression of biomarker 1 OR not elevated expression of biomarker 2 = not elevated, otherwise missing;
Biomarker Algorithm (gap.0): not high biomarker 4 expression and not high biomarker 3 expression and pdvar not elevated = TRUE

| | coef | exp(coef) | se(coef) | Z | Pr(>\|z\|) |
|---|---|---|---|---|---|
| RT TRUE | 0.3823 | 1.4657 | 0.5020 | 0.762 | 0.446 |

| | exp(coef) | exp(−coef) | lower .95 | upper .95 |
|---|---|---|---|---|
| RT TRUE | 1.4657 | 0.6823 | 0.5480 | 3.921 |

Concordance = 0.515 (se = 0.058)
Likelihood ratio test = 0.77 on 2 df, p = 0.7
Wald test = 0.72 on 2 df, p = 0.7
Score (logrank) test = 0.73 on 2 df, p = 0.7

TABLE 14C
Significant benefit of RT for DFS for patients nonlinear algorithm “gap.0”
false; non-linear biomarker algorithm “gap.0” uses biomarker 1 “stromal
PD1/stromal surface area” and biomarker 2 tumoral IRF9 H-SCORE and biomarker
3 tumoral ALDHA1 H-SCORE and biomarker 4 tumoral GLUT1 H-SCORE
Patients: n = 450, number of events = 86
(4 observations deleted due to missingness)
Patients: from all study sites, node negative, treated with BCS, not treated with chemotherapy,
not HER2+, not triple negative, and biomarker algorithm complete
Patient subset: biomarker algorithm gap.0 = FALSE
Outcome: either ipsilateral invasive breast event or distant invasive event or contralateral invasive breast event
Analysis: Cox Proportional Hazard Analysis
Biomarker 1: number of cells in stromal compartment with PD1 expression divided by area of stromal compartment
Biomarker 2: H-SCORE of IRF9 expression in tumor compartment
Biomarker 3: H-SCORE of ALDHA1 expression in tumor compartment
Biomarker 4: H-SCORE of GLUT1 expression in tumor compartment
Elevated Expression (biomarker 1): biomarker 1 expression > 25 percentile of biomarker 1 expression for population
Elevated Expression (biomarker 2): biomarker 2 expression > 25 percentile of biomarker 2 expression for population
High Expression (biomarker 3): biomarker 3 expression > 75 percentile of biomarker 3 expression for population
High Expression (biomarker 4): biomarker 4 expression > 75 percentile of biomarker 4 expression for population
Biomarker Algorithm (pdvar): elevated expression of biomarker 1 AND elevated expression of biomarker 2 = elevated,
not elevated expression of biomarker 1 OR not elevated expression of biomarker 2 = not elevated, otherwise missing;
Biomarker Algorithm (gap.0): not high biomarker 4 expression and not high biomarker 3 expression and pdvar not elevated = TRUE

| | coef | exp(coef) | se(coef) | Z | Pr(>\|z\|) |
|---|---|---|---|---|---|
| RT TRUE | −0.7137 | 0.4898 | 0.2352 | −3.034 | 0.00241 ** |

Signif. codes: 0 ‘***’ 0.001 ‘**’ 0.01 ‘*’ 0.05 ‘.’ 0.1 ‘ ’ 1

| | exp(coef) | exp(−coef) | lower .95 | upper .95 |
|---|---|---|---|---|
| RT TRUE | 0.4898 | 2.042 | 0.3089 | 0.7767 |

Concordance = 0.595 (se = 0.031)
Likelihood ratio test = 11.35 on 2 df, p = 0.003
Wald test = 11.93 on 2 df, p = 0.003
Score (logrank) test = 12.29 on 2 df, p = 0,002

TABLE 14D
Significant benefit of RT for invasive local and distant metastasis free survival for patients
nonlinear algorithm “gap.0” false; non-linear biomarker algorithm “gap.0”
uses biomarker 1 “stromal PD1/stromal surface area” and biomarker 2 tumoral IRF9
H-SCORE and biomarker 3 tumoral ALDHA1 H-SCORE and biomarker 4 tumoral GLUT1 H-SCORE
Patients: n = 369, number of events = 60
(2 observations deleted due to missingness)
Patients: from all study sites, node negative, treated with BCS, not treated with chemotherapy,
not HER2+, not triple negative, and biomarker algorithm complete
Patient subset: biomarker algorithm gap.0 = FALSE
Outcome: either ipsilateral invasive breast event or distant invasive event
Analysis: Cox Proportional Hazard Analysis
Biomarker 1: number of cells in stromal compartment with PD1 expression divided by area of stromal compartment
Biomarker 2: H-SCORE of IRF9 expression in tumor compartment
Biomarker 3: H-SCORE of ALDHA1 expression in tumor compartment
Biomarker 4: H-SCORE of GLUT1 expression in tumor compartment
Elevated Expression (biomarker 1): biomarker 1 expression > 25 percentile of biomarker 1 expression for population
Elevated Expression (biomarker 2): biomarker 2 expression > 25 percentile of biomarker 2 expression for population
High Expression (biomarker 3): biomarker 3 expression > 75 percentile of biomarker 3 expression for population
High Expression {biomarker 4): biomarker 4 expression > 75 percentile of biomarker 4 expression for population
Biomarker Algorithm (pdvar): elevated expression of biomarker 1 AND elevated expression of biomarker 2 = elevated,
not elevated expression of biomarker 1 OR not elevated expression of biomarker 2 = not elevated, otherwise missing;
Biomarker Algorithm (gap.0): not high biomarker 4 expression and not high biomarker 3 expression and pdvar not elevated = TRUE

| | coef | exp(coef) | se(coef) | Z | Pr(>\|z\|) |
|---|---|---|---|---|---|
| RT TRUE | −1.0236 | 0.3593 | 0.2882 | −3.552 | 0.00382 *** |

Signif. codes: 0 ‘***’ 0.001 ‘**’ 0.01 ‘*’ 0.05 ‘.’ 0.1 ‘ ’ 1

| | exp(coef) | exp(−coef) | lower .95 | upper .95 |
|---|---|---|---|---|
| RT TRUE | 0.3593 | 2.783 | 0.2043 | 0.632 |

Concordance = 0.636 (se = 0.033)
Likelihood ratio test = 15.14 on 2 df, p = 5e−04
Wald test = 16.55 on 2 df, p = 3e−04
Score (logrank) test = 17.84 on 2 df, p = 1e−04

### Example 5

A trained healthcare professional obtains a sample provided by a subject. The sample is determined to be non-TNBC (ER and/or PR positive), and not HER2 positive by the healthcare professional, or the sample was already indicated as such. The healthcare professional assays the sample for two or more markers to determine the risk of the recurrence of the breast cancer (e.g. following breast conserving surgery) and the type of care to be provided or de-escalated to the subject to reduce the rate of recurrence—the type of care that is outside of the standard of care recommendations due to the lack of guidance from the standard of care with respect to the markers of interest, these markers including PD-1, IRF9, ALDHA1, and GLUT1.

The expression level of each of these markers is determined by the immunofluorescent labeling of the markers. Employing the imaging modality of MIF, the sample is sectioned and the markers of interest within the sections of the sample are labeled with the immunofluorescent labels. If present in the sample, the label attached on the marker will emit a signal in the visible light spectrum if excited by the fluorescent light at the appropriate wavelength range. Each kind of marker, if their respective label is excited by the appropriate wavelength range, will emit a signal in the visible light spectrum of a different wavelength than another to allow the healthcare professional to interrogate multiple markers, disambiguated by the different emission spectra of their respective label, during the same scan or capture session. That is, if present and under appropriate excitation conditions, the PD-1 label will emit a signal in the visible light spectrum that is distinguishable from the emitted IRF9 label. Likewise, this signal may be obtained when PD-1 and IRF9 are assayed on the same or different slides. Labeling additional markers such as ALDHA1 and GLUT1 with different fluorescent labels having different excitation ranges will permit the assay of multiple labels on the same or different slides.

After obtaining the images showing two or more of the markers that reveal their distribution in the sample, the health care professional delineates each image representing a section of the sample into different compartments. These compartments include tumor, stroma, and may contain other compartments including fat and glass compartments. The protein expression of the cells that are positive for any two or more of the markers in particular compartments provide an indication of a risk of breast cancer recurrence, which then lead to clinical interventions to reduce the risk.

When the percentage of cells that are jointly positive for the stromal PD-1 positive cell count per surface area of the stroma compartment is >25 percentile than a population and level of expression for cells positive for the IRF9 in the tumor compartment is >25 percentile than a population, the population being a plurality of non-TNBC and HER2-negative tumor samples across different tumor grades, age, and tumor sizes, there is an increased risk of recurrence of cancer. Based on the analysis of the images from MIF, there is a significant benefit from RT to reduce this increased risk of recurrence of cancer. Therefore, the subject may be recommended to obtain RT. Alternatively, the subject may be administered RT or an RT boost under guidance from a healthcare professional.

When the biomarkers include ALDHA1 and this biomarker is screened during the MIF panel, then there is a higher risk of recurrence if the sample includes an high level of ALDHA1 expression in the tumor compartment is >75 percentile of the population. Accordingly, the healthcare professional would recommend the patient to be treated with RT. However, if the sample includes an high level of ALDHA1 expression in the tumor compartment is >75 percentile of the population, and there are jointly elevated levels of expression of PD-1 normalized by surface area and IRF9 in certain compartments of the sample (stroma and tumor respectively), then there is an increased risk of recurrence that significantly benefits from RT. Accordingly, the healthcare professional may recommend RT.

When the markers include GLUT1 and this marker is screened during the MIF panel, then there is a higher risk of recurrence if the sample includes an high level of GLUT1 in the tumor compartment is >75 percentile of the population. Accordingly, the healthcare professional would recommend the patient to be treated with RT. However, if the sample includes an high level of GLUT1 in the tumor compartment is >75 percentile of the population, and there are jointly elevated levels of expression of PD-1 normalized by surface area and IRF9 in certain compartments of the sample (stroma and tumor respectively), then there is an increased risk of recurrence and a significant benefit from RT. Accordingly, the healthcare professional may recommend RT.

### Example 6

A patient provides a sample to the healthcare professional for biopsy. The sample is determined to be positive for ER and/or PR and HER2 negative. The patient receives an RT with a boost if 1) the sample does not include jointly elevated levels of expression of stromal PD-1 normalized by stromal surface area and tumor IRF9 biomarkers, such the sample includes levels of expression of stromal PD-1 normalized by stromal surface area and tumor IRF9 that are lower than the elevated levels of expression for the stromal PD-1 and stromal IRF9 and 2) the sample does not have a high level of expression of ALDHA1 in the tumor compartment that is >75 percentile of this biomarker population, such that the sample includes an lower level of ALDHA1 in the tumor compartment that is not >75 percentile of this biomarker population and 3) the sample does not have an elevated level of GLUT1 in the tumor compartment is >75 percentile of this biomarker population, such that the sample has a level of GLUT1 in the tumor compartment that is lower than the 75 percentile of this biomarker population and 4) other risk factors including, but not limited to, lymphovascular invasion (LVI), young age, or high grade.

### ADDITIONAL NOTES

TABLE 15

| Biomarker | | | Recommended |
|---|---|---|---|
| algorithm | Standard of Care | Case | Treatment |
| Nonlinear algorithm | Women over 65 years of | Both elevated (see Table 9) | RT |
| pdvar: IRF9 AND | age† or 70 years or older | Elevated stromal PD1 | |
| stromal PD1/stromal | with Stage I, ER- positive | expression/stromal tumor | |
| area | breast cancer after BCS | area (>25 percentile of this | |
| | can Omit RT‡. | biomarker population) | |
| | | AND elevated IRF9 | |
| | | expression quantity (>25% | |
| | | population) | |
| Nonlinear algorithm | Women over 65 years of | All elevated or high (see | RT |
| not.glut1.pdvar.0 - IRF9 | age† or 70 years or older | Table 12B) | |
| AND stromal | with Stage I, ER- positive | High GLUT1 expression in | |
| PD1/stromal area AND | breast cancer after BCS | tumor cells (>75 percentile | |
| GLUT1 | can Omit RT‡. | of this biomarker | |
| | | population) AND | |
| | | (Elevated stromal PD1 | |
| | | expression/stromal tumor | |
| | | area (>25 percentile of this | |
| | | biomarker population) | |
| | | AND elevated IRF9 | |
| | | expression quantity (>25 | |
| | | percentile of this biomarker | |
| | | population)) | |
| Nonlinear- IRF9 AND | Women over 65 years of | All elevated or high (see | RT |
| stromal PD1/stromal | age† or 70 years or older | Table 12A) High ALDHA1 | |
| area AND ALDHA11 | with Stage I, ER- positive | expression in tumor cells | |
| | breast cancer after BCS | (>75 percentile of this | |
| | can Omit RT‡. | biomarker population), | |
| | | AND Elevated stromal PD1 | |
| | | expression/stromal tumor | |
| | | area (>25 percentile of this | |
| | | biomarker population) | |
| | | AND elevated IRF9 | |
| | | expression quantity (>25 | |
| | | percentile of this biomarker | |
| | | population) | |
| Nonlinear- IRF9 AND | Women over 65 years of | All elevated or high (see | RT |
| stromal PD1/stromal | age† or 70 years or older | Table 13) High ALDHA1 | |
| area AND ALDHA1 | with Stage I, ER- positive | expression in tumor cells | |
| AND GLUT1 | breast cancer after BCS | (>75 percentile of this | |
| | can Omit RT‡. | biomarker population), | |
| | | High GLUT1 expression in | |
| | | tumor cells (>75 percentile | |
| | | of this biomarker | |
| | | population), AND Elevated | |
| | | stromal PD1 | |
| | | expression/stromal tumor | |
| | | area (>25 percentile of this | |
| | | biomarker population) | |
| | | AND elevated IRF9 | |
| | | expression quantity (>25 | |
| | | percentile of this biomarker | |
| | | population) | |
| Nonlinear- IRF9 AND | Women under 65 years of | None elevated or High; Not | No RT |
| stromal PD1/stromal | age OR 70 years are | High GLUT1 expression in | |
| area AND ALDHA1 | normally recommended | tumor cells (>75 percentile | |
| AND GLUT1 | RT | of this biomarker | |
| | | population), Not High | |
| | | ALDHA1 expression (<25 | |
| | | percentile of this biomarker | |
| | | population), AND not | |
| | | (Elevated stromal PD1 | |
| | | expression/stromal tumor | |
| | | area (>25 percentile of this | |
| | | biomarker population) | |
| | | AND elevated IRF9 | |
| | | expression quantity (>25 | |
| | | percentile of this biomarker | |
| | | population)) | |
| | | AND no other higher risk | |
| | | factors (eg., young age <50 | |
| | | years, higher tumor grade, | |
| | | larger tumor size > T1) | |
| Nonlinear- IRF9 AND | Women under 65 years of | not [symbol] high level of GLUT1 | No RT AND |
| stromal PD1/stromal | age OR 70 years are | in the tumor compartment | (Mastectomy or |
| area AND GLUT1 | normally recommended | of the sample of >75 | systemic therapy |
| | RT | percentile [symbol] of a population | or chemotherapy) |
| | | and [symbol] not jointly elevated | |
| | | levels of expression of PD- | |
| | | 1 and IRF9 in the sample | |
| Nonlinear- IRF9 AND | Women under 65 years of | None elevated or High; Not | No RT AND |
| stromal PD1/stromal | age OR 70 years are | High GLUT1 expression in | (Mastectomy of |
| area AND ALDHA1 | normally recommended | tumor cells (>75 percentile | systemic therapy |
| AND GLUT1 | RT | of this biomarker | or chemotherapy) |
| | | population), Not High | |
| | | ALDHA1 expression (<25 | |
| | | percentile of this biomarker | |
| | | population), AND not | |
| | | (Elevated stromal PD1 | |
| | | expression/stromal tumor | |
| | | area (>25 percentile of this | |
| | | biomarker population) | |
| | | AND elevated IRF9 | |
| | | expression quantity (>25 | |
| | | percentile of this biomarker | |
| | | population)) | |
| | | And other high risk factors | |
| | | (eg., young age <50 years, | |
| | | higher tumor grade, larger | |
| | | tumor size > T1) | |
| Nonlinear- IRF9 AND | Women under 65 years of | Not High GLUT1 | No RT |
| stromal PD1/stromal | age OR 70 years are | expression in tumor cells | |
| area AND ALDHA1 | normally recommended | (<25% percentile of this | |
| AND GLUT1 | RT | biomarker population), | |
| | | Elevated ALDHA1 | |
| | | expression (>75 percentile | |
| | | of this biomarker | |
| | | population), Elevated | |
| | | stromal PD1 | |
| | | expression/stromal tumor | |
| | | area (>25 percentile of this | |
| | | biomarker population) | |
| | | AND elevated IRF9 | |
| | | expression quantity (>25 | |
| | | percentile of this biomarker | |
| | | population) | |
| Nonlinear- IRF9 AND | Women under 65 years of | Not High GLUT1 | No RT |
| stromal PD1/stromal | age OR 70 years are | expression in tumor cells | |
| area AND ALDHA1 | normally recommended | (<25% percentile of this | |
| AND GLUT1 | RT | biomarker population), Not | |
| | | High ALDHA1 expression | |
| | | (<25 percentile of this | |
| | | biomarker population), | |
| | | Elevated stromal PD1 | |
| | | expression/stromal tumor | |
| | | area (>2 percentile of this | |
| | | biomarker population) | |
| | | AND elevated IRF9 | |
| | | expression quantity (>25 | |
| | | percentile of this biomarker | |
| | | population) | |
| Nonlinear- IRF9 AND | Women under 65 years of | Elevated GLUT1 | No RT |
| stromal PD1/stromal | age OR 70 years are | expression in tumor cells | |
| area AND ALDHA1 | normally recommended | (>75 percentile of this | |
| AND GLUT1 | RT | biomarker population), | |
| | | Elevated ALDHA1 | |
| | | expression (>75 percentile | |
| | | of this biomarker | |
| | | population), Not Elevated | |
| | | stromal PD1 | |
| | | expression/stromal tumor | |
| | | area (<25 percentile of this | |
| | | biomarker population) | |
| | | AND not elevated IRF9 | |
| | | expression quantity (<25 | |
| | | percentile of this biomarker | |
| | | population) | |
| Nonlinear- IRF9 AND | Women under 65 years of | Not High GLUT1 | RT boost |
| stromal PD1/stromal | age OR 70 years are | expression in tumor cells | |
| area AND ALDHA1 | normally recommended | (>75 percentile of this | |
| AND GLUT1 | RT | biomarker population), Not | |
| | | High ALDHA1 expression | |
| | | (<25 percentile of this | |
| | | biomarker population), | |
| | | AND not (Elevated stromal | |
| | | PD1 expression/stromal | |
| | | tumor area (>25 percentile | |
| | | of this biomarker | |
| | | population) AND elevated | |
| | | IRF9 expression quantity | |
| | | (>25 percentile of this | |
| | | biomarker population)) | |
| | | and have other higher risk | |
| | | factors (eg., higher tumor | |
| | | grade, larger tumor | |
| | | size > T1, Lymphovascular | |
| | | Invasion (LVI) | |

†National Institute for Health and Care Excellence. Early and locally advanced breast cancer: diagnosis and management. Jul. 18, 2018 (www.nice.org.uk/guidance/ng101)
‡Gradishar W J, Anderson B O, Balassanian R, et al. NCCN guidelines insights breast cancer, version 1.2017. J Natl Compr Canc Netw 2017; 15: 433-451.

### Example 7

Immune and metabolic axes can predict RT response in early stage hormone positive HER2 negative breast cancer patients. RT post breast conserving surgery (BCS) reduces in-breast recurrence (IBR) rate in early-stage invasive breast cancer (BC) patients. The RT treatment recommendation is often driven by clinicopathological (CP) factors; however, CP factors alone have limited ability to identify which women significantly benefit from RT, or those with higher IBR risk after BCS plus RT. Biologic factors driving unique phenotypes, in addition to CP, may improve prediction of RT response. In this study we evaluated the role of immune and metabolic signaling axes in predicting RT response in hormone receptor positive, HER2 negative, early-stage BC patients.

Biomarkers from immune and metabolic signaling axes were studied in a cohort of 939 women from Sweden, at a CLIA certified lab (Laguna Hills, CA). Formalin fixed paraffin embedded tissues were assayed for protein expression using multiplex immunofluorescence and multi-spectral imaging. Immune and metabolic axes were assessed using biomarkers combined with a non-linear model, adjusting for patient age. RT prediction by the model was assessed, along and adjusted for CP factors and also among patients over 50-yrs. The model defined patient risk groups that were analyzed for IBR rate using Kaplan Meier analyses and Cox proportional hazards to test for RT-risk group interaction.

Results: Within the cohort, 440 patients had hormone receptor positive, HER2 negative BC treated with BCS (negative margins) and +/−RT without chemotherapy, where 296 patients had complete biomarker data. CP factors individually were not predictive for RT benefit, but grade was prognostic for IBR rate (p=0.02) after BCS without RT. In multivariable analysis, adjusting for CP factors (grade, palpability, continuous size and age), the model was predictive for RT benefit (p-interaction=0.046), identifying patients (n=129) with worse RT benefit (HR=7.8) compared to baseline RT benefit. The model was not prognostic for IBR rate in patients treated with BCS without RT (16% 10-yr IBR rate) but identified patients with increased IBR rates after BCS plus RT (HR=3.9, p<0.001), where corresponding 10-yr IBR rates increased from 3% to 15%. The model was also predictive for RT benefit in women over 50-yrs (p-interaction=0.05). The model identified 28% of women over 50-yrs who had increased IBR rates after BCS plus RT (HR=4.0, p=0.004), where corresponding 10-yr IBR rates increased from 3% to 12%.

Conclusion: The model incorporating metabolic and immune signaling axes assessed in the study was predictive for RT benefit among women with early-stage hormone receptor positive, HER2 negative BC. While CP factors were not predictive of RT benefit, the inclusion of metabolic and immune signaling axes improved identification of patients with high residual risk after BCS plus RT and can potentially aid in personalized treatment of early stage breast cancer based on individualized risk.

In some embodiments, radiotherapy is selected based on marker results from a combination of one or more (including all of): Glut1, CD8, FoxP3 and age.

The described embodiments and examples of the present disclosure are intended to be illustrative rather than restrictive, and are not intended to represent every embodiment or example of the present disclosure, and thus, are not to be limited in scope by the specific embodiments and examples described herein. While the fundamental novel features of the disclosure as applied to various specific embodiments thereof have been shown, described, and pointed out, it will also be understood that various omissions, substitutions, and changes in the details of the methods that are disclosed, may become apparent and may be made by those skilled in the art without departing from the spirit of the disclosure. For example, it is expressly intended that all combinations of those method steps that perform substantially the same function in substantially the same way to achieve the same results are within the scope of the disclosure. Additionally, one or more of the disclosed method steps may be repeated any number of times. Moreover, it should be recognized that method steps shown and/or described in connection with any disclosed form or embodiment of the disclosure may be incorporated in any other disclosed or described or suggested form or embodiment as a general matter of design choice. Further, in at least some of the previously described embodiments, one or more elements used in an embodiment can interchangeably be used in another embodiment unless such a replacement is not technically feasible. Further, various modifications and variations can be made without departing from the spirit or scope of the disclosure as set forth in the following claims both literally and in equivalents recognized in law.

What is claimed is:

1. A method of determining a risk of recurrence for breast cancer, the method comprising:
obtaining a sample provided by a subject, wherein the sample is characterized as estrogen receptor (ER) and/or progesterone receptor (PR) positive, and human epidermal growth factor 2 (HER2) negative;
assaying the sample for markers, the markers comprising programmed cell death protein 1 (PD-1) and interferon regulatory factor 9 (IRF9); and
assessing the subject as having a significant risk of recurrence if the assaying step indicates elevated levels of expression of PD-1 and IRF9 in the sample;
assessing the subject as having no significantly increased risk of recurrence if the assaying step indicates levels of expression lower than the elevated levels of expression for the PD-1 and IRF9.

2. The method of claim 1, further comprising delineating the sample into compartments, the compartments comprising tumor, stroma, fat, and glass compartments.

3. The method of claim 1, further comprising recommending radiotherapy (RT) to the subject if the assaying step indicates the elevated levels of expression of PD-1 and IRF9 in the sample.

4. The method of claim 1, further comprising recommending an RT boost to the subject if the assaying step indicates the elevated levels of expression of PD-1 and IRF9 in the sample.

5. The method of claim 3 or 4, wherein the elevated levels of expression of PD-1 in the sample comprises a percentage of cells that are positive for the PD-1 per surface area of the stroma region,
wherein the percentage of cells that are positive for the PD-1 per surface area of the stroma compartment is >25 percentile than a population.

6. The method of any one of claim 3 or 4, wherein the elevated levels of expression of IRF9 in the sample comprises a percentage of cells that are positive for the IRF9 per surface area of the tumor compartment,
wherein the percentage of cells that are positive for the IRF9 per surface area of the tumor compartment is >25 percentile than a population.

7. The method of claim 5, wherein the elevated levels of expression of IRF9 in the sample comprises a percentage of cells that are positive for IRF9 per surface area of the tumor compartment,
wherein the percentage of cells that are positive for the IRF9 per surface area of the tumor compartment is >25 percentile than the population.

8. The method of claim 1, wherein the markers further comprise aldehyde dehydrogenase A1 (ALDHA1).

9. The method of claim 8, further comprising assessing the subject as having higher risk of recurrence if the assaying step indicates an elevated level of ALDHA1 in the tumor compartment is >75 percentile of a population.

10. The method of claim 9, further comprising recommending RT to the subject if the assaying step indicates the elevated level of ALDHA1.

11. The method of claim 9, further comprising recommending a RT boost to the subject if the assaying step indicates the elevated level of ALDHA1 subject already underwent RT or is currently undergoing RT.

12. The method of claim 8, further comprising assessing the subject as having increased risk of recurrence and a significant benefit to RT if the assaying step indicates an elevated level of ALDHA1 in the tumor compartment of the sample of >75 percentile of a population and the elevated levels of expression of PD-1 and IRF9 in the sample.

13. The method of claim 8, further comprising recommending de-intensification of the RT if the assaying step indicates an elevated level of ALDHA1 in the tumor compartment of the sample of >75 percentile of a population and not the elevated levels of expression of PD-1 and IRF9 in the sample.

14. The method of claim 12 or 13, wherein the elevated levels of expression of PD-1 in the sample comprises a number of cells that are positive for the PD-1 per surface area of the stroma compartment,
wherein the number of cells that are positive for the PD-1 per surface area of the stroma compartment is >25 percentile than the population,
wherein the elevated levels of expression of IRF9 in the sample comprises a percentage of cells that are positive for the IRF9 in the tumor compartment, and
wherein the percentage of cells that are positive for the IRF9 per surface area of the tumor compartment is >25 percentile than the population.

15. The method of claim 1, wherein the markers further comprise glucose transporter 1 (GLUT1).

16. The method of claim 15, further comprising assessing the subject as having decreased risk of recurrence but no significant benefit to RT if the assaying step indicates a non-elevated level of GLUT1 in the tumor compartment of the sample is not >75 percentile of a population and the non-elevated levels of expression of PD-1 and IRF9 in the sample.

17. The method of claim 15, further comprising recommending de-intensification of the RT if the assaying step indicates an elevated level of GLUT1 in the tumor compartment of the sample of >75 percentile of a population and the elevated levels of expression of PD-1 and IRF9 in the sample.

18. The method of claim 16 or 17 wherein the elevated levels of expression of PD-1 in the sample comprises a number of cells that are positive for the PD-1 per surface area of the stroma compartment,
wherein the number of cells that are positive for the PD-1 per surface area of the stroma compartment is >25 percentile than the population,
wherein the elevated levels of expression of IRF9 in the sample comprises a percentage of cells that are positive for the IRF9 per surface area of the tumor compartment, and
wherein the percentage of cells that are positive for the IRF9 per surface area of the tumor compartment is >25 percentile than the population.

19. The method of any one of claims 5-7, 9-14, 16-18, wherein the population comprises levels of the markers from a plurality of samples from different individuals, the plurality of samples including multiple tumor grades, multiple individual ages, and multiple sizes of the samples.

20. The method of any one of claims 1-19, wherein the assaying step comprises:
obtaining images, from at least a portion of the sample, of the markers, wherein the markers are indicated by light emission from excitation of immunofluorescent labels specific for each of the markers, and
counting the markers displayed on the obtained images.

21. The method of claim 20, wherein the obtaining of the images step is performed using multiplex immunofluorescence (MIF).

22. The method of any one of the preceding claims, wherein the markers are interrogated together on the same one or more substantially planar slices of the sample.

23. The method of any one of the preceding claims, comprising using a non-linear model to analyze an interactivity between the markers.

24. The method of any one of the preceding claims, wherein the subject is older than 50 years of age.

25. The method of claim 24, wherein the subject is older than 55 years of age.

26. The method of any one of the preceding claims, wherein a status of the subject is node-negative.

27. The method of any one of claims 1-25, wherein a status of the subject is node-positive.

28. The method of any one of the preceding claims, wherein the markers further comprise ER, PR, and HER2.

29. A method for treating breast cancer, the method comprising:
obtaining a sample provided by a subject, wherein the sample is characterized as ER and PR positive, and HER2 negative;
assaying the sample for markers, the markers comprising PD-1 and IRF9; and
providing an RT boost to the subject if the assaying step indicates elevated levels of expression of PD-1 and IRF9 in the sample; and
de-escalating the RT of the subject if the assaying step indicates levels of expression lower than the elevated levels of expression for the PD-1 and IRF9.

30. The method of claim 29, further comprising delineating the sample into compartments, the compartments comprising tumor, stroma, fat, and glass compartments.

31. The method of claim 29 or 30, wherein the elevated levels of expression of PD-1 in the sample comprises a percentage of cells that are positive for the PD-1 per surface area of the stroma region,
wherein the percentage of cells that are positive for the PD-1 per surface area of the stroma compartment is >25 percentile than a population.

32. The method of claim 29 or 30, wherein the elevated levels of expression of IRF9 in the sample comprises a percentage of cells that are positive for the IRF9 per surface area of the tumor compartment,
wherein the percentage of cells that are positive for the IRF9 per surface area of the tumor compartment is >25 percentile than a population.

33. The method of claim 31, wherein the elevated levels of expression of IRF9 in the sample comprises a percentage of cells that are positive for IRF9 per surface area of the tumor compartment,
wherein the percentage of cells that are positive for the IRF9 per surface area of the tumor compartment is >25 percentile than the population.

34. The method of claim 30, wherein the markers further comprise aldehyde dehydrogenase A1 (ALDHA1).

35. The method of claim 34, further comprising treating the subject with the RT due to a higher risk of recurrence if the assaying step indicates an elevated level of ALDHA1 in the tumor compartment is >75 percentile of a population.

36. The method of claim 34, further comprising treating the subject with an RT boost if the assaying step indicates the elevated level of ALDHA1 subject already underwent RT or is currently undergoing RT.

37. The method of claim 34, further comprising de-intensifying the RT to the subject if the assaying step indicates an elevated level of ALDHA1 in the tumor compartment of the sample of >75 percentile of a population and the elevated levels of expression of PD-1 and IRF9 in the sample.

38. The method of claim 37, wherein the elevated levels of expression of PD-1 in the sample comprises a percentage of cells that are positive for the PD-1 per surface area of the stroma compartment,
wherein the percentage of cells that are positive for the PD-1 per surface area of the stroma compartment is >25 percentile than the population,
wherein the elevated levels of expression of IRF9 in the sample comprises a percentage of cells that are positive for the IRF9 per surface area of the tumor compartment, and
wherein the percentage of cells that are positive for the IRF9 per surface area of the tumor compartment is >25 percentile than the population.

39. The method of claim 30, wherein the markers further comprise glucose transporter 1 (GLUT1).

40. The method of claim 39, further comprising de-intensifying the RT to the subject if the assaying step indicates an elevated level of GLUT1 in the tumor compartment of the sample of >75 percentile of a population and the elevated levels of expression of PD-1 and IRF9 in the sample.

41. The method of claim 40, wherein the elevated levels of expression of PD-1 in the sample comprises a percentage of cells that are positive for the PD-1 per surface area of the stroma compartment,
wherein the percentage of cells that are positive for the PD-1 per surface area of the stroma compartment is >25 percentile than the population,
wherein the elevated levels of expression of IRF9 in the sample comprises a percentage of cells that are positive for the IRF9 per surface area of the tumor compartment, and
wherein the percentage of cells that are positive for the TRF9 per surface area of the tumor compartment is >25 percentile than the population.

42. The method of any one of claims 31-33, 35-38, 40, and 41, wherein the population comprises levels of the markers from a plurality of samples from different individuals, the plurality of samples including multiple tumor grades, multiple individual ages, and multiple sizes of the samples.

43. The method of any one of claims 30-42, wherein the assaying step comprises:
obtaining images, from at least a portion of the sample, of the markers, wherein the markers are indicated by light emission from excitation of immunofluorescent labels specific for each of the markers, and
counting the markers displayed on the obtained images.

44. The method of claim 43, wherein the obtaining of the images step is performed using MIF.

45. The method of any one of claims 29-44, wherein the markers are interrogated together on the same one or more substantially planar slices of the sample.

46. The method of any one of claims 29-45, comprising using a non-linear model to analyze an interactivity between the markers.

47. The method of any one of claims 29-46, wherein the subject is older than 50 years of age.

48. The method of claim 47, wherein the subject is older than 55 years of age.

49. The method of any one of claims 29-48, wherein a status of the subject is node-negative.

50. The method of any one of claims 29-48, wherein a status of the subject is node-positive.

51. The method of any one of the claims 29-50, wherein the markers further comprise ER, PR, and HER2.

52. A method for treating breast cancer, the method comprising:
providing a sample, wherein the sample is characterized as ER and PR positive, and HER2 negative;
receiving an RT boost if the sample includes elevated levels of expression of PD-1 and IRF9; and
de-escalating an RT if the sample includes levels of expression of PD-1 and IRF9 that lower than the elevated levels of expression for the PD-1 and IRF9.

53. The method of claim 52, the sample is delineated into compartments, the compartments comprising tumor, stroma, fat, and glass compartments.

54. The method of claim 52 or 53, wherein the elevated levels of expression of PD-1 in the sample comprises a percentage of cells that are positive for the PD-1 per surface area of the stroma region,
wherein the percentage of cells that are positive for the PD-1 per surface area of the stroma compartment is >25 percentile than a population.

55. The method of claim 52 or 53, wherein the elevated levels of expression of IRF9 in the sample comprises a percentage of cells that are positive for the IRF9 per surface area of the tumor compartment,
wherein the percentage of cells that are positive for the IRF9 per surface area of the tumor compartment is >25 percentile than a population.

56. The method of claim 55, wherein the elevated levels of expression of IRF9 in the sample comprises a percentage of cells that are positive for IRF9 per surface area of the tumor compartment,
wherein the percentage of cells that are positive for the IRF9 per surface area of the tumor compartment is >25 percentile than the population.

57. The method of claim 53, wherein the markers further comprise aldehyde dehydrogenase A1 (ALDHA1).

58. The method of claim 57, further comprising receiving the RT due to a higher risk of recurrence if the sample includes an elevated level of ALDHA1 in the tumor compartment is >75 percentile of a population.

59. The method of claim 58, further comprising receiving an RT boost if already had the RT or is currently undergoing the RT.

60. The method of claim 57, further comprising de-intensifying the RT if the sample includes an elevated level of ALDHA1 in the tumor compartment of >75 percentile of a population and the elevated levels of expression of PD-1 and TRF9.

61. The method of claim 60, wherein the elevated levels of expression of PD-1 in the sample comprises a percentage of cells that are positive for the PD-1 per surface area of the stroma compartment,
wherein the percentage of cells that are positive for the PD-1 per surface area of the stroma compartment is >25 percentile than the population,
wherein the elevated levels of expression of IRF9 in the sample comprises a percentage of cells that are positive for the IRF9 per surface area of the tumor compartment, and
wherein the percentage of cells that are positive for the IRF9 per surface area of the tumor compartment is >25 percentile than the population.

62. The method of claim 53, wherein the markers further comprise glucose transporter 1 (GLUT1).

63. The method of claim 62, further comprising de-intensifying the RT if the sample includes an elevated level of GLUT1 in the tumor compartment of the sample of >75 percentile of a population and the elevated levels of expression of PD-1 and IRF9.

64. The method of claim 63, wherein the elevated levels of expression of PD-1 in the sample comprises a percentage of cells that are positive for the PD-1 per surface area of the stroma compartment,
wherein the percentage of cells that are positive for the PD-1 per surface area of the stroma compartment is >25 percentile than the population,
wherein the elevated levels of expression of IRF9 in the sample comprises a percentage of cells that are positive for the IRF9 per surface area of the tumor compartment, and
wherein the percentage of cells that are positive for the IRF9 per surface area of the tumor compartment is >25 percentile than the population.

65. The method of any one of claims 54-56, 58-61, 63, and 64, wherein the population comprises levels of the markers from a plurality of samples from different individuals, the plurality of samples including multiple tumor grades, multiple individual ages, and multiple sizes of the samples.

66. The method of any one of claims 52-65, wherein the markers further comprise ER, PR, and HER2.
